(19) United States
(12) Patent Application Publication
(10) Pub. No.: US 2003/0082677 A1
(43) Pub. Date: May 1, 2003

(54) Novel EDIRF, MTR-1, LSP-1, TAP-1, and PA-I molecules and uses therefor

(76) Inventors: Douglas A. Holtzman, Jamaica Plain, MA (US); Yang Pan, Bellevue, WA (US); Sean A. McCarthy, San Diego, CA (US); David P. Gearing, Victoria (AU)

Correspondence Address: Intellectual Property Group, MILLENNIUM PHARMACEUTICALS, INC., 75 Sidney Street, Cambridge, MA, 02139, US

(21) Appl. No.: 09/774,381

(22) Filed: Jan. 30, 2001

Related U.S. Application Data
(63) Continuation-in-part of application No. 08/941,354, filed on Sep. 30, 1997, now abandoned.
(63) Continuation-in-part of application No. 09/010,674, filed on Jan. 22, 1998, now abandoned.
(63) Continuation-in-part of application No. 09/014,347, filed on Jan. 27, 1998, now abandoned.
(63) Continuation-in-part of application No. 09/474,151, filed on Dec. 28, 1999, now Pat. No. 6,267,003.
Application No. 09/474,151 is a continuation of application No. 09/004,206, filed on Jan. 8, 1998, now abandoned.
Application No. 09/474,151 is a continuation of application No. 09/483,414, filed on Jan. 14, 2000, now abandoned.
Application No. 09/483,414 is a continuation of application No. 09/213,571, filed on Dec. 18, 1998, now abandoned.
Application No. 09/213,571 is a continuation-in-part of application No. 08/994,890, filed on Dec. 19, 1997, now abandoned.
(60) Provisional application No. 60/061,149, filed on Oct. 6, 1997.
(60) Provisional application No. 60/061,159, filed on Oct. 6, 1997.
(60) Provisional application No. 60/061,143, filed on Oct. 6, 1997.

(51) Int. Cl. C07K 14/705; C07H 21/04; C12P 21/02; C12N 5/06

(52) U.S. Cl. 435/69.1; 435/320.1; 435/325; 530/350; 536/23.2

(57) ABSTRACT

Novel EDIRF, MTR-1, LSP-1, TAP-1, and PA-I polypeptides, proteins, and nucleic acid molecules are disclosed. In addition to isolated, full-length EDIRF, MTR-1, LSP-1, TAP-1, and PA-I proteins, the invention further provides isolated EDIRF, MTR-1, LSP-1, TAP-1, and PA-I fusion proteins, antigenic peptides and anti-EDIRF, MTR-1, LSP-1, TAP-1, and PA-I antibodies. The invention also provides EDIRF, MTR-1, LSP-1, TAP-1, and PA-I nucleic acid molecules, recombinant expression vectors containing a nucleic acid molecule of the invention, host cells into which the expression vectors have been introduced and non-human transgenic animals in which an EDIRF, MTR-1, LSP-1, TAP-1, and PA-I gene has been introduced or disrupted. Diagnostic, screening and therapeutic methods utilizing compositions of the invention are also provided.

### RELATED APPLICATIONS

This application is a continuation-in-part of U.S. patent application Ser. No. 08/941,354, filed Sep. 30, 1997 (pending).

This application is also a continuation-in-part of U.S. patent application Ser. No. 09/010,674, filed Jan. 22, 1998 (pending), which claims the benefit of U.S. Provisional Application Serial No. 60/061,149, filed Oct. 6, 1997 (expired).

This application is also a continuation-in-part of U.S. patent application Ser. No. 09/014,347, filed Jan. 27, 1998 (pending), which claims the benefit of U.S. Provisional Application Serial No. 60/061,159 filed Oct. 6, 1997 (expired).

This application is also a continuation-in part of U.S. patent application Ser. No. 09/474,151, filed Dec. 21, 2000, which is a continuation of U.S. patent application Ser. No. 09/004,206, filed Jan. 8, 1998 (abandoned), which claims the benefit of U.S. Provisional Application Serial No. 60/061,143, filed Oct. 6, 1997 (expired).

This application is also a continuation-in-part of U.S. patent application Ser. No. 09/483,414, filed Jan. 14, 2000 (pending), which is a continuation of U.S. patent application Ser. No. 09/213,571, filed Dec. 18, 1998 (abandoned), which is a continuation-in-part of U.S. patent application Ser. No. 08/994,890, filed Dec. 19, 1997 (abandoned). The entire content of each of the above-referenced patent applications is incorporated herein by this reference.

### BACKGROUND OF THE INVENTION

Cytokines are small peptide molecules produced by a variety of cells that mediate a wide range of biological activities. Arai, K.-I. et al. (1990) Annu. Rev. Biochem. 59:783 and Paul, W. E. and R. A. Seder (1994) Cell 76:241. Through a complex network, cytokines regulate fUnctions including cellular growth, inflammation, immunity, differentiation and repair. Mosmann, T. R. (1991) Curr. Opin. Immunol. 3:311. One family of cytokines, termed interleukin-17 (IL-17), has been identified which plays a role in regulation of immune function. IL-17 family members identified to date include human IL-17 (see Yao et al. (1995) J. Immunol. 155:5483-5486 and Fossiez et al. (1996) J. Exp. Med. 183:2593-2603), murine IL-17 (see Yao et al. (1996) Gene 168:223-225), alternatively known as cytotoxic T lymphocyte associated antigen-8 (CTLA-8), rat IL-17, also known as CTLA-8 (see Rouvier et al (1993) J. Immunol. 150:5445-5456), and a viral IL-17 known as Herpesvirus Saimiri gene 13 (HSV13) (see Albrecht et al. (1992) J. Virol. 66(8):5047-5058 and Yao et al. (1995) Immunity 3:811-821.) The IL-17 cytokines are known to induce stromal cells to secrete cytokines involved in inflammatory and hematopoietic processes. For example, IL-17 has been shown to induce secretion of IL-6, IL-8, PGE2, and G-CSF from primary cultures of synovial fibroblasts as well as inducing IL-6 secretion in six additional stromal cell lines tested. Fossiez et al. (1996) J. Exp. Med. 183:2593-2603. The induction of IL-6 secretion by fibroblasts has been correlated with both activation of NF-κB and induction of surface expression of intracellular adhesion molecule-1 (ICAM-1) as well as with a mitogenic response in T cells. Yao et al. (1996) Gene 168:223-225 and Yao et al. (1995) Immunity 3:811-821. Furthermore, IL-17 has been implicated indirectly in the hematopoiesis of neutrophils. Fossiez et al. (1996) J. Exp. Med. 183:2593-2603.

Given the important role of cytokines, such as the IL-17 cytokines, in regulation of the cellular immune response, inflammation and hematopoietic processes, there exists a need for the identification of novel IL-17-like molecules as well as for modulators of such molecules for use in regulating a variety of cellular responses.

The transporter superfamily encompasses a large number of genes whose protein products are involved in the transport of a variety of hexoses and other carbon compounds. Members of this superfamily include numerous bacterial sugar-proton symporters, bacterial transporters of carboxylic acid, sugar transporters of various yeasts and high energy glucose transporters in higher plants. In addition, the mammalian glucose transporter proteins, Glut 1, Glut 2, Glut 3, Glut 4, Glut 5 and Glut 7, which mediate the exchange of glucose between the blood and the cytoplasm of cells, have been identified as members of this superfamily.

A common characteristic of the proteins of this superfamily is the presence of 12 transmembrane segments. The transmembrane domains of several members of this transporter superfamily are arranged so that both the N- and C-termini are located at the cytoplasmic surface. Conserved motifs in the N terminal half of members of this superfamily usually demonstrate similar motifs in the corresponding C terminal half. Based on the presence of conserved motifs at symmetric positions in both halves of the transporter molecule, it has been proposed that these proteins are descended from a common ancestor comprised of six transmembrane segments whose gene underwent a duplication event (Mueckler (1994) Eur. J. Biochem. 219:713-725). In addition, it has been suggested that these motifs may be conserved among members of the transmembrane segment transporter superfamily in order to maintain the conformational stability of the proteins and may, also, be involved with salt-bridging between the helices.

Several of the members of the transport superfamily have been linked to metabolic disorders. For example, defects in the transport of glucose across the plasma membrane of cells has been implicated as underlying diseases such as diabetes mellitus. Studies have demonstrated a potential link between the role of GLUT 2 in insulin secretion and insulin-dependent diabetes mellitus. (Orci, 1. et al. (1990) J. Clin. Invest. 86:1615-1622). In addition, defective translocation of GLUT 4 in muscle cells has been demonstrated to be a factor leading to insulin resistance associated with obesity and non-insulin dependent diabetes mellitus. (Kelley, D. et al. (1996) J. Clin. Invest. 97(12):2705-2713).

Cells of the immune response characteristically express a variety of cell-surface proteins which are crucial to proper functioning of the immune system. Such proteins include surface immunoglobulins, non-immunoglobulin cell surface antigen receptors, cellular adhesion molecules, as well as other selected phenotypic markers. Many of these cell surface proteins are members of the immunoglobulin (Ig)superfamily of proteins, characterized by the existence of at least one immunoglobulin (Ig) domain. Such proteins function in an variety of immune cell functions ranging from immune cell development and differentiation, antigen recognition, antibody production, cellular signal transduction, and cellular homing of immune responsive cells from the circulation to cites of increased antigen concentration.

In some instances, the diversified nature of immune cell function can be attributed to the specific pattern of expression of such cell surface proteins. For example, cells expressing VCAM proteins of the Ig superfamily are known to be involved primarily in cellular adhesion, whereas T lymphocytes characteristically express distinct patterns of the phenotypic markers, CD4, CD3, and CD8. Given importance of such cell surface proteins in the proper functioning of the immune system, there exists a need to identify novel cell-surface molecules which function to regulate the immune response and whose aberrant function can lead to immune response disorders such as congenital or acquired immunodeficiency, and or inflammatory disorders such as arthritis.

The differentiation of hematopoietic stem cells (HSCs) involves a series of lineage commitment steps accompanied by the acquisition of specific phenotypic characteristics (Huang, S. and Terstappen, L. (1992) Nature 360: 745-49). Cells gain or lose antigenic features and responsiveness to specific cytokines and growth factors based on their lineage and stage of differentiation. As development proceeds, HSCs become committed to specific myeloid, lymphoid or erythroid lineages. These committed “progenitor” stem cells ultimately differentiate into a wide variety of specialized cell types which include erythrocytes, neutrophils, basophils, eosinophils, platelets, mast cells, monocytes, tissue macrophages, osteoclasts, and the T and B lymphocytes.

Red blood cells (erythrocytes), white blood cells (leucocytes) and platelets (thrombocytes) are the predominant cell-types in the blood. Platelets are derived from detached fragments of larger cells called megakaryocytes which reside predominantly in the bone marrow. Megakaryopoiesis and platelet production are central to the release of cytokines, wound healing and blood coagulation. The failure of an organism to maintain adequate megakaryocyte numbers lends to thrombocytopenia and consequent bleeding disorders that can, in the extreme, result in death. Several humoral factors have been shown to promote megakaryocyte and platelet development, including interleukin-1 (IL-1) (Schmidt, J. A., J. Exp. Med 160:772-787, 1984; March, C. J. et al., Nature 315:641-647, 1985); IL-3 (Yang, Y. C. et al., Cell 47:3-10, 1986; Ikebuchi, K. et al., Proc. Natl. Acad. Sci. USA 84:9035-9039, 1987), IL-6 (Hirano, T., et al., Proc. Natl. Acad. Sci. USA 82:5490-5494, 1985; Hirano, T., et al., Nature 324:73-76,1986; Ishibashi, T. et al., Proc. Natl. Acad. Sci. USA 86:5953-5957, 1989); IL-11 (Paul, S. R. et al., Proc. Natl. Acad. Sci. USA 87:7512-7516, 1990; Teramura, M. et al., Blood 79:327-331, 1992), leukemia inhibitory factor (Metcalf, D. et al., Blood 77:2150-2153, 1991), granulocyte-macrophage colony-stimulating factor (Wong, G., et al., Science 228:810-815, 1985), erythropoietin (Miyake, T. et al., J. Biol. Chem. 252:5558-5564, 1977; Jacobs, K. et al., Nature 313:806-815, 1985), and stem cell factor (Hendrie, P. C. et al., Exp. Hematol. 19:1031-1037, 1991). However, most of these factors are pleiotropic and consequently their roles in physiological replication of thrombocyte poiesis are unclear. Additional activities implicated in megakaryopoiesis include megakaryocyte-potentiating factor (Yamaguchi, N. et al., J. Biol. Chem. 269:805-808, 1994), megakaryocyte stimulatory factor (Tayrien, G., and Rosenberg, R. D., J. Biol. Chem. 262:3262-3268, 1987; Greenberg, S. M. et al., Exp. Hematol. 19:1031-1037, 1987), megakaryocyte colony-stimulating factor (Ogata, K. et al., Int. J. Cell Cloning 8:103-120, 1990; Erikson-Miller, C. L. et al., Blood Cell Growth Factors: Their Present and Future Use in Hematology and Oncology, Alpha Med Press, pp. 204-220, 1992; Erikson-Miller, C. L. et al., Br. J. Haematol. 84:197-203, 1993), thrombopoiesis-stimulating factor (McDonald, T. P. et al., J. Lab. Clin. Med. 85:59-66, 1975), and thrombopoietin (Hill, R. and Levin, J. Exp. Hematol. 14:752-759, 1986; Hill, R. J. et al., J. Exp. Hematol 20:354-360, 1992). Sources for these activities have included the urine, serum or plasma from aplastic and/or thrombocytopenic humans (McDonald, T. P., Biochem. Med. 13:101-110, 1975; Ogata, K. et al., Int. J. Cell Cloning 8:103-120, 1990), rats (Odell, T. T. et al., Proc. Soc. Exp. Biol. Med. 108:428-431, 1961), rabbits (Evatt, B. L. et al., J. Lab. Clin. Med. 83:364-371, 1974; Hill, R. J. et al., Exp. Hematol. 20:354-360, 1992), and dogs (Mazur, E. and South, K., Exp. Hematol. 13:1164-1172, 1985).

Recent studies have implicated the ligand of the c-mpl cytokine receptor, thrombopoietin (TPO), as a megakaryocyte lineage-specific factor (Bartley, T. D. et al. 1994; Cell Vol. 77:1117-1124; de Sauvage, F. J., et al. (1994) Vol. 369:533-38; Gurney, A. L. (1995) Blood Vol. 85 (4):981-88; Sohma, Y. et al. (1994) FEBS Letters 353:47-61). The human TPO cDNA encodes a mature protein of approximately 332 amino acids that can be divided into two domains: an amino terminal domain of 153 amino acids with homology to erythropoietin and a unique C-terminal domain of 175 amino acids containing multiple N-linked glycosylation sites. Both recombinant full-length human TPO and a truncated form consisting of the EPO-like domain (TPO₁₅₃) stimulate [³H]-thymidine incorporation in murine BaF3 cells transfected with human c-mpl, demonstrating that he epo-like domain alone is sufficient for activation of c-mpl (deSauvage F. J. et al. (1994) Nature 369:533). Recombinant human TPO stimulated human megakaryocytopoiesis in vitro alone or in the presence of other exogenously added early-acting hematopoietic growth factors (deSauvage F. J. et al., Nature 369:533, 1994; Bartley, T. D. et al., Cell 77:1117, 1994; Kaushansky K. et al., Nature 369:568, 1994; Wendling F., Nature 369:571, 1994) Also, TPO stimulated platelet production in mice and dramatically increased the number of megakaryocytes in the spleen and bone marrow, indicating that TPO regulates thrombopoiesis in vivo (deSauvage F. J. et al., Nature 369:533, 1994; Lok S. et al., Nature 369:565, 1994; Kaushansky K. et al., Nature 369:568, 1994; Wendling F., Nature 369:571, 1994).

The identification of novel secreted factors which are involved in stem cell proliferation and differentiation is critical to the understanding of these biological processes and in the development of new therapeutic compositions and methods of using the same.

The placenta is the source of several peptide hormones that are homologous to hormones synthesized in other endocrine tissues. These placental hormones, which belong to the prolactin-growth hormone superfamily, are believed to play crucial roles in normal fetal development. Members of the prolactin-growth hormone superfamily include mouse placental lactogen I (mPL-I), mouse placental lactogen II (mPL-II), which bind to the prolactin receptor, and other proteins like mouse proliferin (PLF) (Linzer D. I. H. et al., Proc. Natl. Acad. Sci. U.S.A. (1985) 82:4356; Lee S. J. et al., Endocrinology (1988) 122:1761), mouse proliferin-related protein (PRP) (Linzer D. I. H. and Nathans D., EMBO J. 4:1419; Colosi P. et al., Mol. Endocrinol. (1988) 2:579), rat PL-I variant (Deb S. et al., J. Biol. Chem. (1991) 266:1605-1610), and rat PRL-like proteins (PLP) A (Campbell W. J. et al., Endocrinology (1989) 125:1565-1574), B (Ogilvie S. et al., Endocrinology (1990) 126:2561-2566), and C (Deb S. et al., Endocrinology (1991) 128:3066-3072; Deb S. et al., J. Biol. Chem. (1991) 266:23027-23032). PLF was discovered as a serum growth factor-inducible mRNA (Linzer D. I. H. and Nathans D., Proc. Natl. Acad. Sci. U.S.A. (1983) 80:4271; ibid. (1984) 81:4255) and protein (Nilsen-Hamilton M. et al., Cell (1980) 20:19) in mouse fibroblasts, and expression of PLF in muscle cells has been shown to inhibit muscle cell-specific gene expression and differentiation (Wilder E. L. and Linzer D. I. H., Mol. Cell. Biol. (1989) 9:430; Muscat G. E. O. et al., Mol. Endocrinol. (1991) 5:802). The PRP mRNA was detected in placenta as a cDNA clone which cross-hybridized to the PLF cDNA (Linzer D. I. H. and Nathans D., EMBO J. 4:1419). All of these proteins show significant structural similarity (Southard J. N., Molecular and Cellular Endocrinoilogy (1991), 79: C133-C140) and are produced by the same trophoblast giant cells (Yamaguchi M., Program of the 75th Annual Meeting of the Endocrine Society, Las Vegas (1993), p. 113 abstract), but their biological activities and gestational profiles in the maternal blood differ. The known biological activities of mPL-I and mPL-II are prolactin-like. The functions of PRP and PLF are not understood, but both have been postulated to be involved in regulating the initiation and then the cessation of placental neovascularization (Jackson D. et al., Science (1994) 266:1581-1584).

### SUMMARY OF THE INVENTION

The present invention is based, at least in part, on the discovery of novel molecules referred to herein as “Embryo-Derived Interleukin Related Factor I and II” (“EDIRF”, e.g., human “EDIRF I” (also referred to herein as “TANGO 89”) and “EDIRF II” and mouse “EDIRF I”); “transmembrane segment superfamily” (“MTR-1”), also referred to herein as “TANGO 49”; “Leukocyte-Specific Protein-1” (“LSP-1”), also referred to herein as “HOMEDEPO” or “TANGO 111”; “Thrombopoietin Analog Protein” (“TAP-1”), also referred to herein as “TANGO 94”; and “Proliferin Analog I” (“PA-I”), also referred to herein as “MOPAI” or “TANGO 95”, nucleic acid and protein molecules. The EDIRF, MTR-1, LSP-1, TAP-1, and PA-I molecules of the present invention are useful as modulating agents in regulating a variety of cellular processes. Accordingly, in one aspect, this invention provides isolated nucleic acid molecules encoding EDIRF, MTR-1, LSP-1, TAP-1, and PA-I proteins or biologically active portions thereof, as well as nucleic acid fragments suitable as primers or hybridization probes for the detection of EDIRF, MTR-1, LSP-1, TAP-1, and PA-I -encoding nucleic acids.

In one embodiment, an EDIRF, MTR-1, LSP-1, TAP-1, or PA-I nucleic acid molecule is 60%, 65%, 70%, 75%, 80%, 85%, 90%, or 95% homologous to the nucleotide sequence shown in SEQ ID NO:1, 3, 5, 34, 43, 49, or 53, the nucleotide sequence of the DNA insert of the plasmid deposited with ATCC as Accession Number 98613, 98614, 98550, or 98554, or a complement thereof. In another embodiment, an EDIRF, MTR-1, LSP-1, TAP-1, or PA-I nucleic acid molecule is 60%, 65%, 70%, 75%, 80%, 85%, 90%, or 95% homologous to the nucleotide sequence shown in SEQ ID NO:7, 8, 9, 36, 45, 51, or 55, or a complement thereof. In a preferred embodiment, an isolated EDIRF, MTR-1, LSP-1, TAP-1, or PA-I nucleic acid molecule encodes the amino acid sequence of human or mouse EDIRF, MTR-1, LSP-1, TAP-1, or PA-I, respectively.

In a preferred embodiment, an isolated EDIRF nucleic acid molecule has the nucleotide sequence of SEQ ID NO:7 or a complement thereof. In another embodiment, an EDIRF nucleic acid molecule further comprises nucleotides 1-68 of SEQ ID NO:1. In yet another preferred embodiment, an EDIRF nucleic acid molecule further comprises nucleotides 609-738 of SEQ ID NO:1.

In another preferred embodiment of the invention, an isolated EDIRF nucleic acid molecule has the nucleotide sequence of SEQ ID NO:8 or a complement thereof. In another embodiment, an EDIRF nucleic acid molecule further comprises nucleotides 1-73 of SEQ ID NO:3. In yet another preferred embodiment, an EDIRF nucleic acid molecule further comprises nucleotides 613-756 of SEQ ID NO:3.

In yet another preferred embodiment of the invention, an isolated EDIRF nucleic acid molecule has the nucleotide sequence of SEQ ID NO:9 or a complement thereof. In another embodiment, an EDIRF nucleic acid molecule further comprises nucleotides 1-128 of SEQ ID NO:5. In yet another preferred embodiment, an EDIRF nucleic acid molecule further comprises nucleotides 912-1259 of SEQ ID NO:5.

In another preferred embodiment of the invention, an isolated MTR-1 nucleic acid molecule has the nucleotide sequence of SEQ ID NO:36 or a complement thereof. In another embodiment, an MTR-1 nucleic acid molecule further comprises nucleotides 1-56 of SEQ ID NO:34. In yet another preferred embodiment, an MTR-1 nucleic acid molecule further comprises nucleotides 1925-3689 of SEQ ID NO:34.

In another preferred embodiment of the invention, an isolated LSP-1 nucleic acid molecule has the nucleotide sequence of SEQ ID NO:45 or a complement thereof. In another embodiment, an LSP-1 nucleic acid molecule further comprises nucleotides 1-1331 of SEQ ID NO:43. In yet another preferred embodiment, an LSP-1 nucleic acid molecule further comprises nucleotides 2010-2462 of SEQ ID NO:43.

In yet another preferred embodiment of the invention, an isolated TAP-1 nucleic acid molecule has the nucleotide sequence of SEQ ID NO:51 or a complement thereof. In another embodiment, a TAP-1 nucleic acid molecule further comprises nucleotides 259-528 of SEQ ID NO:49.

In still another preferred embodiment of the invention, an isolated PA-I nucleic acid molecule has the nucleotide sequence of SEQ ID NO:55 or a complement thereof. In another embodiment, a PA-I nucleic acid molecule further comprises nucleotides 1-54 of SEQ ID NO:53. In yet another preferred embodiment, a PA-I nucleic acid molecule further comprises nucleotides 817-933 of SEQ ID NO:43.

In yet another preferred embodiment, an isolated EDIRF, MTR-1, LSP-1, TAP-1, or PA-I nucleic acid molecule has the nucleotide sequence shown SEQ ID NO:1, 3, 5, 34, 43, 49, or 53, the nucleotide sequence of the DNA insert of the plasmid deposited with ATCC as Accession Number 98613, 98614, 98550, or 98554, or a complement thereof.

In another embodiment, an EDIRF, MTR-1, LSP-1, TAP-1, or PA-I nucleic acid molecule includes a nucleotide sequence encoding a protein having an amino acid sequence sufficiently homologous, as defined herein, to the amino acid sequence of SEQ ID NO:2, 4, 6, 35, 44, 50, or 54. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 40% homology, preferably 50% homology, more preferably 60%-70% homology across the amino acid sequences of the domains and contain at least one, preferably two, and more preferably three or four structural domains, are defined herein as sufficiently homologous. Furthermore, amino acid or nucleotide sequences that share at least 40%, preferably 50%, more preferably 60, 70, or 80% homology and share a common functional activity are defined herein as sufficiently homologous.

In yet another embodiment, an EDIRF, MTR-1, LSP-1, TAP-1, or PA-I nucleic acid molecule includes a nucleotide sequence encoding a protein having an amino acid sequence at least 60%, 65%, 70%, 75%, 80%, 85%, 90%, or 95% homologous to the amino acid sequence of SEQ ID NO:2, 4, 6, 35, 44, 50, or 54. In a preferred embodiment, an EDIRF, MTR-1, LSP-1, TAP-1, or PA-I nucleic acid molecule includes a nucleotide sequence encoding a protein having the amino acid sequence of SEQ ID NO:2, 4, 6, 35, 44, 50, or 54.

In another embodiment, an isolated nucleic acid molecule of the present invention encodes an EDIRF protein which includes a signal sequence, an N-terminal unique domain, a C-terminal IL-17-like domain, and is secreted. In another embodiment, an isolated nucleic acid molecule of the present invention encodes an MTR-1 protein which includes twelve transmembrane domains and a pentameric motif. In another embodiment, an isolated nucleic acid molecule of the present invention encodes an LSP-1 protein which includes a signal sequence, an N-terminal immunoglobulin (Ig)-like domain, a C-terminal transmembrane domain, and is membrane bound or secreted. In another embodiment, an isolated nucleic acid molecule of the present invention encodes a TAP-1 protein which includes a serine-proline-threonine-rich domain, a C-terminal unique domain and is secreted. In yet another embodiment, an isolated nucleic acid molecule of the present invention includes a TAP-1 carboxy-terminal domain. In another embodiment, an isolated nucleic acid molecule of the present invention encodes a PA-I protein which includes a cysteine-rich domain and is secreted.

In another embodiment, the EDIRF, MTR-1, LSP-1, TAP-1, or PA-I nucleic acid molecule encodes an EDIRF, MTR-1, LSP-1, TAP-1, or PA-I protein and is a naturally occurring nucleotide sequence. In yet another embodiment, an isolated nucleic acid molecule of the present invention encodes an EDIRF, MTR-1, LSP-1, TAP-1, or PA-I protein and comprises a nucleotide sequence which hybridizes under stringent hybridization conditions to a nucleic acid molecule having the nucleotide sequence of SEQ ID NO:1, 3, 5, 7, 8, 9, 34, 36, 43, 45, 49, 51, 53, or 55, or the DNA insert of the plasmid deposited with ATCC as Accession Number 98613, 98614, 98550, or 98554.

Another embodiment of the invention features EDIRF, MTR-1, LSP-1, TAP-1, and PA-I nucleic acid molecules which specifically detect EDIRF, MTR-1, LSP-1, TAP-1, and PA-I nucleic acid molecules relative to nucleic acid molecules encoding other EDIRF, MTR-1, LSP-1, TAP-1, or PA-I family members. For example, in one embodiment, an EDIRF nucleic acid molecule hybridizes under stringent conditions to a nucleic acid molecule comprising nucleotides 520-738 of SEQ ID NO:1, nucleotides 525-756 of SEQ ID NO:3, nucleotides 1-350 or to nucleotides 1025-1259 of SEQ ID NO:5. In another embodiment, a TAP-1 nucleic acid molecule hybridizes under stringent conditions to a nucleic acid molecule comprising nucleotides 1-528 of SEQ ID NO:49 or to nucleotides 1 to 258 of SEQ ID NO:51. In another embodiment, a PA-I nucleic acid molecule hybridizes under stringent conditions to a nucleic acid molecule comprising nucleotides 654-795 of SEQ ID NO:53, or nucleotides 600-741 of SEQ ID NO:55.

In another embodiment, the invention provides an isolated EDIRF, MTR-1, LSP-1, TAP-1, or PA-I nucleic acid molecule which comprises at least 15 consecutive (e.g., at least 15 contiguous) nucleotides of SEQ ID NO:1, 3, 5, 7, 8, 9, 34, 36, 43, 45, 49, 51, 53, or 55.

Another embodiment of the invention provides an isolated nucleic acid molecule which is antisense to the coding strand of an EDIRF, MTR-1, LSP-1, TAP-1, or PA-I nucleic acid molecule.

Another aspect of the invention provides a vector comprising an EDIRF, MTR-1, LSP-1, TAP-1, or PA-I nucleic acid molecule. In certain embodiments, the vector is a recombinant expression vector. In another embodiment the invention provides a host cell containing a vector of the invention. The invention also provides a method for producing EDIRF, MTR-1, LSP-1, TAP-1, or PA-I protein by culturing in a suitable medium, a host cell of the invention containing a recombinant expression vector such that EDIRF, MTR-1, LSP-1, TAP-1, or PA-I protein is produced. In one embodiment, the host cell is a non-human mammalian host cell (e.g., a CHO cell or a COS cell).

Another aspect of this invention features isolated or recombinant EDIRF, MTR-1, LSP-1, TAP-1, and PA-I proteins and polypeptides. In one embodiment, an isolated EDIRF protein has a signal sequence, an N-terminal unique domain, a C-terminal IL-17-like domain, and is secreted. In another embodiment, an isolated MTR-1 protein includes twelve transmembrane domains and a pentameric motif. In addition, the MTR-1 protein of the invention can further include at least one octameric motif, preferably two. In a further aspect of the invention, the MTR-1 protein includes twelve transmembrane domains, a pentameric motif and an extended loop segment between the sixth and seventh transmembrane domains. In another embodiment, an isolated LSP-1 protein has a signal sequence, an N-terminal immunoglobulin (Ig)-like domain, a C-terminal transmembrane domain, and is membrane bound or secreted. In another embodiment, an isolated TAP-1 protein has a serine-proline-threonine rich region, a C-terminal unique domain and is secreted. In another embodiment, an isolated PA-I protein has a cysteine-rich domain and is secreted.

In another embodiment, an isolated EDIRF, MTR-1, LSP-1, TAP-1, or PA-I protein has an amino acid sequence sufficiently homologous to the amino acid sequence of SEQ ID NO:2, 4, 6, 35, 44, 50, or 54. In a preferred embodiment, an EDIRF, MTR-1, LSP-1, TAP-1, or PA-I protein has an amino acid sequence at least about 60%, 65%, 70%, 75%, 80%, 85%, 90%, or 95% homologous to the amino acid sequence of SEQ ID NO:2, 4, 6, 35, 44, 50, or 54. In another embodiment, an EDIRF, MTR-1, LSP-1, TAP-1, or PA-I protein has the amino acid sequence of SEQ ID NO:2, 4, 6, 35, 44, 50, or 54. In yet another embodiment, an isolated EDIRF, MTR-1, LSP-1, TAP-1, or PA-I protein is a cell surface-associated protein.

Another embodiment of the invention features an isolated EDIRF, MTR-1, LSP-1, TAP-1, or PA-I protein which is encoded by a nucleic acid molecule having a nucleotide sequence at least about 60%, 65%, 70%, 75%, 80%, 85%, 90%, or 95% homologous to a nucleotide sequence of SEQ ID NO:7, 8, 9, 36, 45, 51, or 55, the nucleotide sequence of the DNA insert of the plasmid deposited with ATCC as Accession Number 98613, 98614, 98550, or 98554, or a complement thereof. Another embodiment of the invention features an isolated EDIRF, MTR-1, LSP-1, TAP-1, or PA-I protein which is encoded by a nucleic acid molecule having a nucleotide sequence at least about 60%, 65%, 70%, 75%, 80%, 85%, 90%, or 95% homologous to a nucleotide sequence of SEQ ID NO:1, 3, 5, 34, 43, 49, or 53, the nucleotide sequence of the DNA insert of the plasmid deposited with ATCC as Accession Number 98613, 98614, 98550, or 98554, or a complement thereof. This invention also features an isolated EDIRF, MTR-1, LSP-1, TAP-1, or PA-I protein which is encoded by a nucleic acid molecule having a nucleotide sequence which hybridizes under stringent hybridization conditions to a nucleic acid molecule comprising the nucleotide sequence of SEQ ID NO:7, 8, 9, 36, 45, 51, or 55, or a complement thereof. The invention further features an isolated EDIRF, MTR-1, LSP-1, TAP-1, or PA-I protein which is encoded by a nucleic acid molecule having a nucleotide sequence which hybridizes under stringent hybridization conditions to a nucleic acid molecule comprising the nucleotide sequence of SEQ ID NO:1, 3, 5, 34, 43, 49, or 53, the nucleotide sequence of the DNA insert of the plasmid deposited with ATCC as Accession Number 98613, 98614, 98550, or 98554, or a complement thereof.

In another embodiment, the invention provides an isolated EDIRF, MTR-1, LSP-1, TAP-1, or PA-I protein which comprises at least 10 consecutive (e.g., at least 10 contiguous) amino acid residues of SEQ ID NO:2, 4, 6, 35, 44, 50, or 54. In another embodiment, the invention provides an isolated EDIRF, MTR-1, LSP-1, TAP-1, or PA-I protein which corresponds to at least half of a full-length amino acid sequence of SEQ ID NO:2, 4, 6, 35, 44, 50, or 54.

The EDIRF, MTR-1, LSP-1, TAP-1, and PA-I proteins of the present invention, or biologically active portions thereof, can be operatively linked to a non-EDIRF, non-MTR-1, non-LSP-1, non-TAP-1, or non-PA-I polypeptide to form EDIRF, MTR-1, LSP-1, TAP-1, and PA-I fusion proteins. The invention further features antibodies that specifically bind EDIRF, MTR-1, LSP-1, TAP-1, or PA-I proteins, such as monoclonal or polyclonal antibodies. In addition, the EDIRF, MTR-1, LSP-1, TAP-1, and PA-I proteins or biologically active portions thereof can be incorporated into pharmaceutical compositions, which optionally include pharmaceutically acceptable carriers.

In another aspect, the present invention provides a method for detecting EDIRF, MTR-1, LSP-1, TAP-1, or PA-I expression in a biological sample by contacting the biological sample with an agent capable of detecting an EDIRF, MTR-1, LSP-1, TAP-1, or PA-I nucleic acid molecule, protein or polypeptide such that the presence of EDIRF, MTR-1, LSP-1, TAP-1, or PA-I nucleic acid molecule, protein or polypeptide is detected in the biological sample.

In another aspect, the present invention provides a method for detecting the presence of EDIRF, MTR-1, LSP-1, TAP-1, or PA-I activity in a biological sample by contacting the biological sample with an agent capable of detecting an indicator of EDIRF, MTR-1, LSP-1, TAP-1, or PA-I activity such that the presence of EDIRF, MTR-1, LSP-1, TAP-1, or PA-I activity is detected in the biological sample.

In another aspect, the invention provides a method for modulating EDIRF, MTR-1, LSP-1, TAP-1, or PA-I activity comprising contacting the cell with an agent that modulates EDIRF, MTR-1, LSP-1, TAP-1, or PA-I activity such that EDIRF, MTR-1, LSP-1, TAP-1, or PA-I activity in the cell is modulated. In one embodiment, the agent inhibits EDIRF, MTR-1, LSP-1, TAP-1, or PA-I activity. In another embodiment, the agent stimulates EDIRF, MTR-1, LSP-1, TAP-1, or PA-I activity. In one embodiment, the agent is an antibody that specifically binds to EDIRF, MTR-1, LSP-1, TAP-1, or PA-I protein. In another embodiment, the agent modulates expression of EDIRF, MTR-1, LSP-1, TAP-1, or PA-I by modulating transcription of an EDIRF, MTR-1, LSP-1, TAP-1, or PA-I gene or translation of an EDIRF, MTR-1, LSP-1, TAP-1, or PA-I mRNA. In yet another embodiment, the agent is a nucleic acid molecule having a nucleotide sequence that is antisense to the coding strand of the EDIRF, MTR-1, LSP-1, TAP-1, or PA-I mRNA or the EDIRF, MTR-1, LSP-1, TAP-1, or PA-I gene.

In one embodiment, the methods of the present invention are used to treat a subject having a disorder characterized by aberrant EDIRF, MTR-1, LSP-1, TAP-1, or PA-I protein or nucleic acid expression or activity by administering an agent which is an EDIRF, MTR-1, LSP-1, TAP-1, or PA-I modulator to the subject. In one embodiment, the EDIRF, MTR-1, LSP-1, TAP-1, or PA-I modulator is an EDIRF, MTR-1, LSP-1, TAP-1, or PA-I protein. In another embodiment the EDIRF, MTR-1, LSP-1, TAP-1, or PA-I modulator is an EDIRF, MTR-1, LSP-1, TAP-1, or PA-I nucleic acid molecule. In yet another embodiment, the EDIRF, MTR-1, LSP-1, TAP-1, or PA-I modulator is a peptide, peptidomimetic, or other small molecule. In a preferred embodiment, the disorder characterized by aberrant EDIRF, MTR-1, LSP-1, TAP-1, or PA-I protein or nucleic acid expression is an immune or inflammatory disorder, a hematopoietic disorder, a differentiative or developmental disorder, a proliferative disorder, or a proinflammatory disorder.

The present invention also provides a diagnostic assay for identifying the presence or absence of a genetic alteration characterized by at least one of (i) aberrant modification or mutation of a gene encoding an EDIRF, MTR-1, LSP-1, TAP-1, or PA-I protein; (ii) mis-regulation of said gene; and (iii) aberrant post-translational modification of an EDIRF, MTR-1, LSP-1, TAP-1, or PA-I protein, wherein a wild-type form of said gene encodes an protein with an EDIRF, MTR-1, LSP-1, TAP-1, or PA-I activity.

In another aspect the invention provides a method for identifying a compound that binds to or modulates the activity of an EDIRF, MTR-1, LSP-1, TAP-1, or PA-I protein, by providing a indicator composition comprising an EDIRF, MTR-1, LSP-1, TAP-1, or PA-I protein having EDIRF, MTR-1, LSP-1, TAP-1, or PA-I activity, contacting the indicator composition with a test compound, and determining the effect of the test compound on EDIRF, MTR-1, LSP-1, TAP-1, or PA-I activity in the indicator composition to identify a compound that modulates the activity of an EDIRF, MTR-1, LSP-1, TAP-1, or PA-I protein.

Other features and advantages of the invention will be apparent from the following detailed description and claims.

### BRIEF DESCRIPTION OF THE DRAWINGS

FIG. 1 depicts the cDNA sequence and predicted amino acid sequences of two forms of human EDIRF. FIG. 1A depicts the nucleotide and amino acid sequences of human EDIRF I. The nucleotide sequence corresponds to nucleic acids 1 to 738 of SEQ ID NO:1. The amino acid sequence corresponds to amino acids 1 to 180 of SEQ ID NO:2. FIG. 1B depicts the nucleotide and amino acid sequence of human EDIRF II. The nucleotide sequence corresponds to nucleic acids 1 to 1259 of SEQ ID NO:5. The amino acid sequence corresponds to amino acids 1 to 261 of SEQ ID NO:6.

FIG. 2 depicts the cDNA sequence and predicted amino acid sequence of murine EDIRF I. The nucleotide sequence corresponds to nucleic acids 1 to 756 of SEQ ID NO:3. The amino acid sequence corresponds to amino acids 1 to 180 of SEQ ID NO:4.

FIG. 3 depicts an alignment of the amino acid sequences of human EDIRF I (corresponding to amino acids 1 to 180 of SEQ ID NO:2), human EDIRF II (corresponding to amino acids 1-261 of SEQ ID NO:6), murine EDIRF I (corresponding to amino acids 1 to 180 of SEQ ID NO:4), human IL-17 precursor (Swiss-Prot™ Accession No. Q16552, corresponding to amino acid residues 1-155 of SEQ ID NO:14), murine IL-17 precursor, alternatively known as cytotoxic T lymphocyte-associated antigen 8 precursor or CTLA-8 precursor (Swiss-Prot™ Accession No. Q62386, corresponding to amino acid residues 1-158 of SEQ ID NO:15), and herpesvirus Saimiri immediate early gene 13 protein precursor (Swiss-Prot™ Accession No. P24916, corresponding to amino acid residues 1-157 of SEQ ID NO:16).

FIG. 4 depicts the cDNA sequence and predicted amino acid sequence of murine MTR-1. The nucleic acid sequence corresponds to nucleic acids 1 to 3689 of SEQ ID NO:34. The amino acid sequence corresponds to amino acids 1 to 622 of SEQ ID NO:35. The locations of the two octameric motifs are indicated by underlining.

FIG. 5 depicts an alignment of the amino acid sequence of murine MTR-1 (corresponding to amino acids 495 to 622 of SEQ ID NO:35) and human glucose transporter 3 (SEQ ID NO:39).

FIG. 6 depicts an alignment of the amino acid sequence of murine MTR-1 (corresponding to amino acids 7 to 617 of SEQ ID NO:35) and beet sugar transporter (SEQ ID NO:40). Murine MTR-1 is shown as the upper sequence and beet sugar transporter is shown as the lower sequence. The locations of the two octameric motifs are indicated by underlining.

FIG. 7 depicts the cDNA sequence and predicted amino acid sequence of human LSP-1. The nucleotide sequence corresponds to nucleic acids 1 to 2462 of SEQ ID NO:43. The amino acid sequence corresponds to amino acids 1 to 226 of SEQ ID NO:44.

FIG. 8 is a schematic drawing depicting selected clones which were isolated and sequenced to derive the nucleotide sequence of the gene encoding human LSP-1. The figure details the relationship between the original, partial LSP-1 clone isolated from a bone marrow cDNA library, three additional clones for which partial sequence information was available, and the final composite sequence generated from complete sequence analysis of the additional clones as well as the nucleotide sequence of the original partial clone. The coding region is depicted by the boxed regions. The shaded box at the N-terminus of the coding region represents the location of the signal peptide (SP), the Ig-like domain (Ig) is depicted as the unshaded box in the center of the coding region, and the C-terminal shaded box represents the transmembrane domain (TM).

FIG. 9 is a schematic diagram depicting the biological and functional domains of human LSP-1. The LSP-1 protein comprises at least a signal peptide from about amino acids 1-20 of the amino acid sequence depicted (which corresponds to the amino acid sequence of SEQ ID NO:44), an Ig-like domain from about amino acids 46-128 of the amino acid sequence depicted, and a transmembrane domain from about amino acids 192-213 of the amino acid sequence depicted.

FIG. 10 depicts an alignment of the LSP-1 nucleic acid molecule with the FDF03 nucleic acid molecule (SEQ ID NO:48; described in International Publication No. WO 98/24906) using the GAP program in the GCG software package (pam120 matrix) and a gap weight of 12 and a length weight of 4. The two sequences are 45.3% identical, with a global alignment score of 1812.

FIG. 11 depicts an alignment of the LSP-1 protein with the FDF03 protein (SEQ ID NO:58; described in International Publication No. WO 98/24906) using the GAP program in the GCG software package (pam120 matrix) and a gap weight of 12 and a length weight of 4. The two sequences are 51.6% identical, with a global alignment score of 291.

FIG. 12 depicts the cDNA sequence and predicted amino acid sequence of human TAP-1. The nucleotide sequence corresponds to nucleic acids 1 to 528 of SEQ ID NO:49. The amino acid sequence corresponds to amino acids 1 to 86 of SEQ ID NO:50.

FIG. 13 depicts an alignment of the amino acid sequences of human TAP-1(corresponding to amino acids 15 to 75 of SEQ ID NO:50) and human TPO (SEQ ID NO:52; Swiss-Prot™ Accession Numbers P40225, 1401246, 939627). Identical amino acids are indicated by a single amino acid code in the row between the TAP-1 and TPO sequences, conserved amino acids are indicated as (+).

FIG. 14 depicts the cDNA sequence and predicted amino acid sequence of murine PA-I. The nucleotide sequence corresponds to nucleic acids 1 to 933 of SEQ ID NO:53. The amino acid sequence corresponds to amino acids 1 to 253 of SEQ ID NO:54.

FIG. 15 depicts an alignment of the amino acid sequences of murine PA-I (corresponding to amino acids 1 to 247 of SEQ ID NO:54), and murine proliferin related protein (SEQ ID NO:56; Swiss-Prot™ Accession No. P04769). The signal sequence is underlined. Cysteine residues are circled, and the location of disufide bonds between certain cysteine residues of PA-I are indicated schematically (-S-S-).

FIG. 16 depicts northern blots performed using mouse PA-I (clone aa014234) as a probe. FIG. 16A depicts a northern blot using human tissue from placenta, heart, brain, lung, liver, skeletal muscle, kidney and pancreas. FIG. 16B depicts a northern blot using tissue from mouse embryos (day 7, 11, 15, and 17 embryos). FIG. 16C depicts a northern blot using human tissue from brain, lung, liver, and kidney.

### DETAILED DESCRIPTION OF THE INVENTION

The present invention is based, at least in part, on the discovery of novel molecules having homology to members of the IL-17 protein family, referred to herein as “EDIRF” protein and nucleic acid molecules, which comprise a family of molecules having certain conserved structural and functional features. The present invention is further based, at least in part, on the discovery of novel molecules of the transmembrane segment transporter superfamily, referred to herein as “MTR-1” nucleic acid and protein molecules, which comprise a family of molecules having certain conserved structural and functional features. The present invention is further based, at least in part, on the discovery of novel molecules specific to peripheral blood leukocytes, referred to herein as “LSP-1” protein and nucleic acid molecules, which comprise a family of molecules having certain conserved structural and functional features. The present invention is further based, at least in part, of the discovery of novel hematopoietic specific factors, referred to herein as “TAP-1” nucleic acid and protein molecules, which comprise a family of molecules having certain conserved structural and functional features. The present invention is still further based, at least in part, on the discovery of novel molecules of the prolactin-growth hormone superfamily, referred to herein as “PA-I” protein and nucleic acid molecules, which comprise a family of molecules having certain conserved structural and functional features.

The term “family” when referring to the protein and nucleic acid molecules of the invention is intended to mean two or more protein or nucleic acid molecules having a common structural domain and having sufficient amino acid or nucleotide sequence homology as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

One embodiment of the invention features EDIRF, MTR-1, LSP-1, TAP-1, and PA-I nucleic acid and protein molecules, preferably human and mouse EDIRF, human LSP-1 and TAP-1, and mouse MTR-1 and PA-I nucleic acid and protein molecules. The EDIRF, MTR-1, LSP-1, TAP-1, and PA-I nucleic acid and protein molecules of the invention are described in further detail in the following subsections.

A. EDIRF Nucleic Acid and Protein Molecules

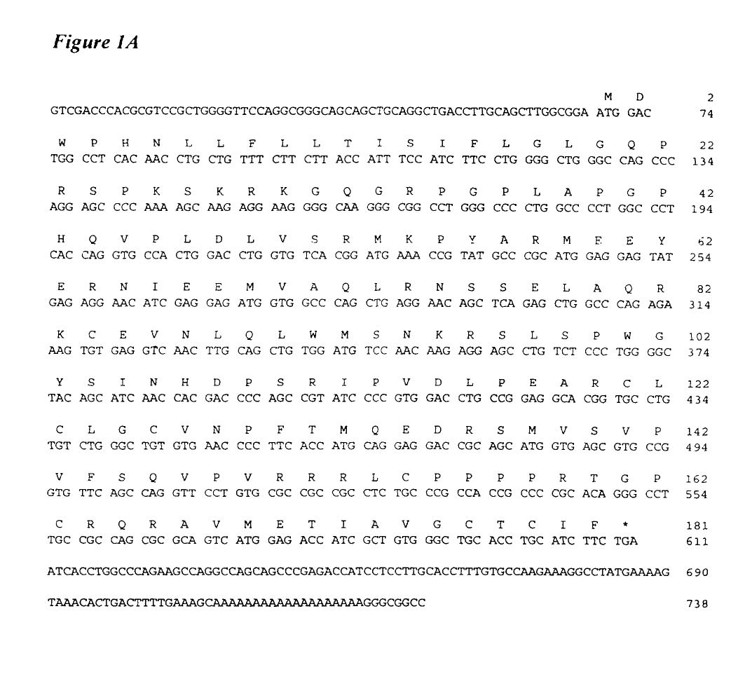

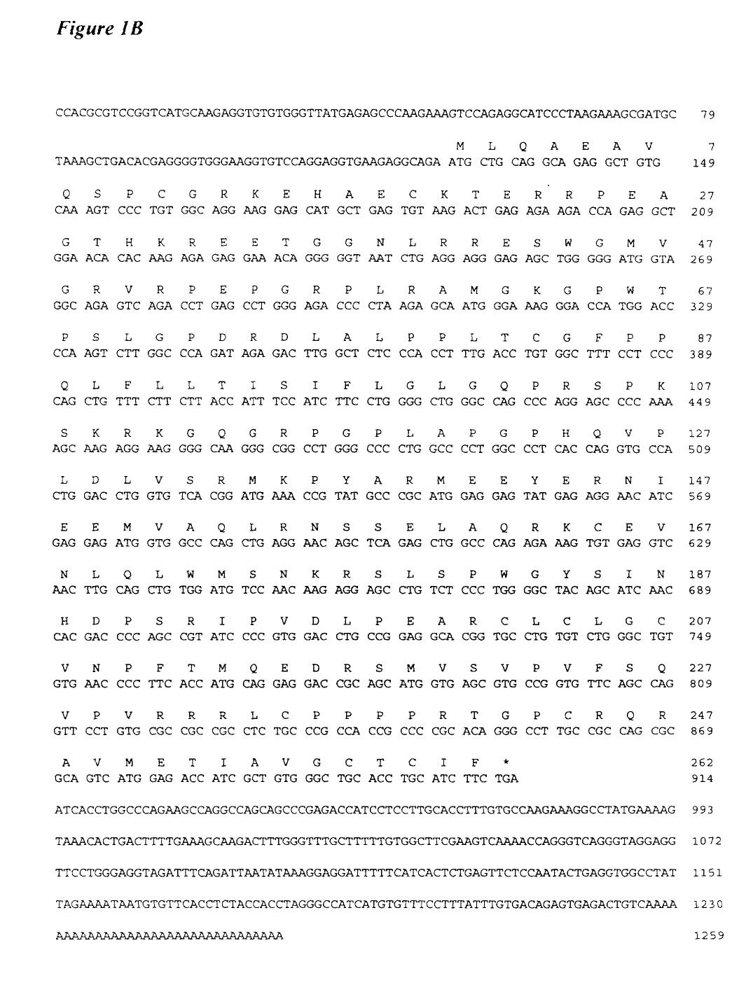

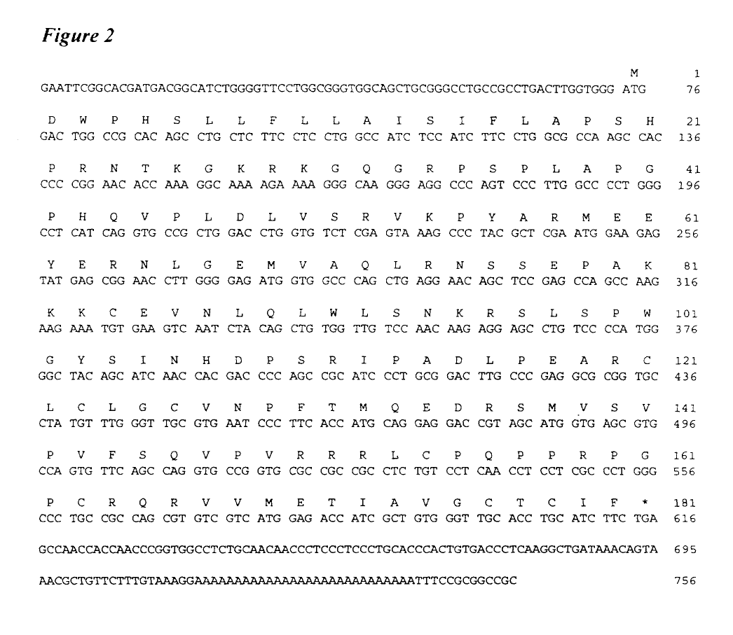

The nucleotide sequences of two human EDIRF nucleic acid molecules (human EDIRF I and human EDIRF II) and the amino acid sequence of the respective human EDIRF protein molecules are depicted in FIG. 1. Human EDIRF II has significant homology to human EDIRF I and includes additional 5′ nucleotide sequence not present in human EDIRF I. The nucleotide sequence of the murine EDIRF I nucleic acid molecule and the amino acid sequence of the murine EDIRF I protein molecule are depicted in FIG. 2.

The EDIRF nucleic acid and protein molecules of the present invention have homology to the IL-17 protein family, whose members include, at least, human IL-17, murine IL-17, alternatively known as cytotoxic T lymphocyte-associated antigen 8 (CTLA-8) and herpesvirus Saimiri immediate early gene 13 protein (HSV13).

In one embodiment, an EDIRF family member is identified based on the presence of a “C-terminal IL-17-like domain” in the protein or corresponding nucleic acid molecule. As used herein, the term “C-terminal IL-17-like domain” refers to a protein domain which is at least about 70-110 amino acid residues in length, preferably at least about 80-100 amino acid residues in length, and more preferably at least about 85-90 amino acid residues in length, and has at least about 25-60%, preferably at least about 30-50%, more preferably at least about 35-45% homology, and even more preferably at least about 38-41% homology with the amino acid sequence of human IL-17, as set forth in SEQ ID NO:14.

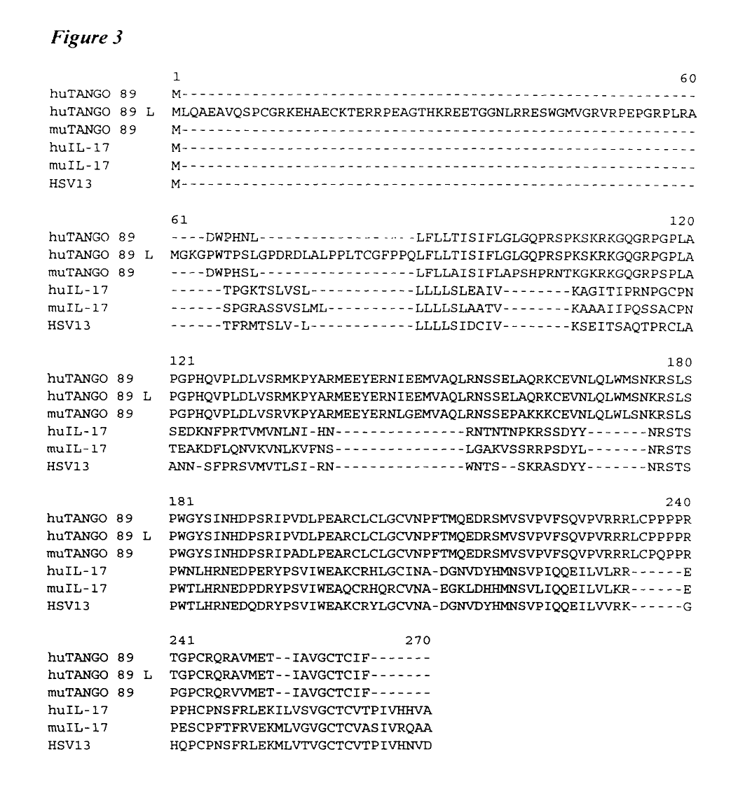

A C-terminal IL-17-like domain further contains at least about three, more preferably at least about four, and even more preferably at least about five cysteine residues which are conserved between EDIRF molecules and members of the IL-17 protein family (e.g., human IL-17, murine IL-17, alternatively known as CTLA-8, and herpesvirus Saimiri immediate early gene 13 protein.) Preferably, the C-terminal IL-17-like domain of EDIRF has cysteine residues which are located in the same or similar positions as cysteine residues in a IL-17 protein family. For example, when an EDIRF protein of the invention is aligned with an IL-17 family member for purposes of comparison (see e.g., FIG. 3) preferred cysteine-rich domains of the invention are those in which cysteine residues in the amino acid sequence of EDIRF are located in the same or similar position as the cysteine residues in the IL-17 family member. As an illustrative embodiment, FIG. 3 shows cysteine residues located in the same or similar positions of the human EDIRF protein (corresponding to SEQ ID NO:2) and human IL-17 at the following locations: amino acid number 121 of human EDIRF I and amino acid number 94 of the human IL-17 protein; amino acid number 126 of human EDIRF I and amino acid number 99 of the human IL-17 protein; amino acid number 163 of human EDIRF I and amino acid number 129 of the human IL-17 protein; amino acid number 178 of human EDIRF I and amino acid number 144 of the human IL-17 protein; and amino acid number 176 of human EDIRF I and amino acid number 146 of the human IL-17 protein.

In a preferred embodiment, a C-terminal IL-17-like domain is located in the C-terminal region of an EDIRF protein. For example, in one embodiment, an EDIRF protein contains a C-terminal IL-17-like domain containing about amino acids 95-180 of SEQ ID NO:2. In another embodiment, an EDIRF protein contains a C-terminal IL-17-like domain containing about amino acids 95-180 of SEQ ID NO:4. In yet another embodiment, an EDIRF protein contains a C-terminal IL-17-like domain containing about amino acids 176-261 of SEQ ID NO:6.

Another embodiment of the invention features EDIRF molecules which contain an N-terminal unique domain. The term “N-terminal unique domain” as used herein, refers to a protein domain of an EDIRF protein family member which includes amino acid residues N-terminal to the N-terminus of a C-terminal IL-17-like domain in the amino acid sequence of the EDIRF protein, e.g., a protein domain which includes amino acid residues from the N-terminus of the C-terminal IL-17-like domain to the N-terminal amino acid residue of the amino acid sequence of the protein. As used herein, an “N-terminal unique domain” refers to a protein domain which is at least about 70-120 amino acid residues in length, preferably at least about 80-110 amino acid residues in length, and more preferably at least about 90-100 amino acid residues in length, and has at least about 65-95%, preferably at least about 70-90%, and more preferably at least about 75-85% homology with the amino acid sequence of a second EDIRF family member. As further defined herein, a N-terminal unique domain of an EDIRF protein family member, however, is not sufficiently homologous to the amino acid sequence of a member of another protein family, such as an IL-17 protein family. For example, an N-terminal unique domain of human EDIRF I (containing about amino acids 1-94 of SEQ ID NO:2 has at least about 80% homology to the N-terminal unique domain of murine EDIRF I (containing about amino acids 1-94 of SEQ ID NO:4) but has no significant homology to the amino acid sequence of human IL-17.

As further defined herein, an N-terminal unique domain may further contain an “N-terminal signal sequence”. As used herein, a “signal sequence” refers to a peptide containing about 20 amino acids which occurs at the extreme N-terminal end of secretory and integral membrane proteins and which contains large numbers of hydrophobic amino acid residues. For example, a signal sequence contains at least about 14-28 amino acid residues, preferably about 16-26 amino acid residues, more preferably about 18-24 amino acid residues, and more preferably about 20-22 amino acid residues, and has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence to a lipid bilayer.

In a preferred embodiment, an EDIRF protein contains both an N-terminal unique domain and a C-terminal IL-17-like domain. In a more preferred embodiment, an EDIRF N-terminal unique domain further contains a signal sequence. In one exemplary embodiment, an EDIRF protein contains an N-terminal unique domain comprising amino acids 1-94 of SEQ ID NO:2 which further contains a signal sequence at about amino acids 1-19, 1-20, 1-21, or preferably, 1-22 of SEQ ID NO:2. In another exemplary embodiment, an EDIRF protein contains an N-terminal unique domain comprising amino acids 1-94 of SEQ ID NO:4 which further contains a signal sequence at amino about acids 1-19, 1-20, 1-21, or preferably, 1-22 of SEQ ID NO:4. In yet another exemplary embodiment, an EDIRF protein contains an N-terminal unique domain comprising amino acids 1-175 of SEQ ID NO:2.

Preferred EDIRF molecules of the present invention have an amino acid sequence sufficiently homologous, as defined herein, to the amino acid sequence of SEQ ID NO:2, SEQ ID NO:4 or SEQ ID NO:6.

As used interchangeably herein an “EDIRF activity”, “biological activity of EDIRF” or “functional activity of EDIRF” refer to an activity exerted by an EDIRF protein, polypeptide or nucleic acid molecule on an EDIRF responsive cell as determined in vivo, or in vitro, according to standard techniques. In one embodiment, an EDIRF activity is a direct activity, such as an association with a cell-surface protein (e.g., an EDIRF receptor). In another embodiment, an EDIRF activity is an indirect activity, such as the induction of synthesis of a second protein (e.g. a cellular cytokine) mediated by interaction of the EDIRF protein with a cell surface protein. In a preferred embodiment, an EDIRF activity is at least one or more of the following activities: (i) interaction of an EDIRF protein in the extracellular millieu with a non-EDIRF protein molecule on the surface of the same cell which secreted the EDIRF protein molecule; (ii) interaction of an EDIRF protein in the extracellular millieu with a non-EDIRF protein molecule on the surface of a different cell from that which secreted the EDIRF protein molecule; (iii) complex formation between an EDIRF protein and an EDIRF receptor; (iv) complex formation between an EDIRF protein and non-EDIRF receptor; and (v) interaction of an EDIRF protein with a second protein in the extracellular millieu. In yet another preferred embodiment, an EDIRF activity is at least one or more of the following activities: (i) activation of an EDIRF-dependent signal transduction pathway; (ii) modulation of secretion of a non-IL-17 cytokine; (iii) interaction of an EDIRF protein with a non-EDIRF cell surface molecule (e.g., a proteoglycan or sulfated proteoglycan); (iv) modulation of secretion of IL-17; (v) modulation of surface expression of a cellular adhesion molecule; (vi) modulation of a proinflammatory cytokine; (vii) modulation of a hematopoietic cytokine; (viii) modulation of the development or differentiation of an EDIRF-expressing cell; (ix) modulation of the development or differentiation of a non-EDIRF-expressing cell; (x) modulation of the homeostasis of an EDIRF-expressing cell; and (xi) modulation of the homeostasis of a non-EDIRF-expressing cell.

Accordingly, another embodiment of the invention features isolated EDIRF proteins and polypeptides having an EDIRF activity. Preferred EDIRF proteins have a C-terminal IL-17-like domain and an EDIRF activity. In another embodiment, the EDIRF protein has an N-terminal unique domain, a C-terminal IL-17-like domain, and an EDIRF activity. In another preferred embodiment, the EDIRF protein has at least at least a C-terminal IL-17-like domain, an N-terminal unique domain, an EDIRF activity, and an amino acid sequence sufficiently homologous to an amino acid sequence of SEQ ID NO:2, SEQ ID NO:4 or SEQ ID NO:6.

In a particularly preferred embodiment, the EDIRF protein and nucleic acid molecules of the present invention are human EDIRF molecules. A human EDIRF cDNA molecule (human EDIRF I) was obtained from a human fetal lung cDNA library as described herein. A second human EDIRF cDNA (human EDIRF II) was also obtained from the library as described herein. The nucleotide sequences of the isolated human EDIRF cDNAs and the predicted amino acid sequences of the human EDIRF proteins are shown in FIG. 1 and in SEQ ID NOs:1 and 2 and SEQ ID NOs:5 and 6, respectively. In addition, the nucleotide sequence corresponding to the coding region of the human EDIRF cDNAs are represented as SEQ ID NOs:7 and 9.

EDIRF mRNA transcripts of approximately <1.0 and ˜5.0 kb were expressed in most human tissues tested (See Example 2).

The human EDIRF I cDNA set forth in SEQ ID NO:1, is approximately 738 nucleotides in length and encodes a protein which is approximately 180 amino acid residues in length (SEQ ID NO:2). The human EDIRF II cDNA set forth in SEQ ID NO:5, is approximately 1259 nucleotides in length and encodes a protein which is approximately 261 amino acid residues in length (SEQ ID NO:6). The human EDIRF proteins contain an N-terminal signal sequence, an N-terminal unique domain, and a C-terminal IL-17-like domain, as defined herein. An EDIRF N-terminal unique domain can be found at least, for example, from about amino acids 1-94 of SEQ ID NO:2 and, for example, from about amino acids 61-175 of SEQ ID NO:6. An EDIRF C-terminal IL-17-like domain can be found at least, for example, from about amino acids 95-180 of SEQ ID NO:2 and, for example, from about amino acids 176-261 of SEQ ID NO:6. A signal sequence can be found at least, for example, from about amino acids 1-20 of SEQ ID NO:2. The prediction of such a signal peptide can be made, for example, utilizing the computer algorithm SIGNALP (Henrik, et al. (1997) Protein Engineering 10:1-6).

In another embodiment, the EDIRF protein and nucleic acid molecules of the present invention are murine EDIRF molecules. A murine EDIRF full-length cDNA molecule (murine EDIRF I) was identified as described herein. The nucleotide sequence of the full-length murine EDIRF I cDNA and the predicted amino acid sequence of the murine EDIRF I protein are shown in FIG. 2 and in SEQ ID NOs:3 and 4, respectively. In addition, the nucleotide sequence corresponding to the coding region of the murine EDIRF I cDNA is represented as SEQ ID NO:8.

EDIRF mRNA transcripts of approximately 1.0 kb were expressed at low levels in adult murine lung (See Example 2). A 1.0 kb developmentally-regulated transcript was also identified in murine embryonic tissue. The expression of murine EDIRF transcripts was also detected by in situ analysis of 13.5-day mouse embryos.

The murine EDIRF I cDNA is approximately 756 nucleotides in length and encodes a protein which is approximately 180 amino acid residues in length (SEQ ID NO:4). The murine EDIRF I protein contains an N-terminal signal sequence, an N-terminal unique domain, and a C-terminal IL-17-like domain, as described herein. An EDIRF N-terminal unique domain can be found at least, for example, from about amino acids 1-94 of SEQ ID NO:4. An EDIRF C-terminal IL-17-like domain can be found at least, for example, from about amino acids 95-180 of SEQ ID NO:4. A signal sequence can be found at least, for example, from about amino acids 1-22 of SEQ ID NO:4. The prediction of such a signal peptide can be made, for example, utilizing the computer algorithm SIGNALP (Henrik, et al. (1997) Protein Engineering 10:1-6).

An alignment of the amino acid sequences of human EDIRF I, human EDIRF II, and murine EDIRF I, as well as human IL-17 (Swiss-Prot™ Accession No. Q16552), murine IL-17, alternatively known as CTLA-8 (Swiss-Prot™ Accession No. Q62386), and herpesvirus Saimiri immediate early gene 13 protein (Swiss-Prot™ Accession No. P24916) is shown in FIG. 3. (The alignment was generated using MegAlign™ sequence alignment software. The initial pairwise alignment step was performed using a Wilbur-Lipmann algorithm with a K-tuple of 2, a GAP penalty of 5, a window of 4, and diagonals saved set to =4. The multiple alignment step was performed using the Clustal algorithm with a PAM 250 residue weight Table, a GAP penalty of 10, and a GAP length penalty of 10.)

B. MTR-1 Nucleic Acid and Protein Molecules

In one embodiment, a member of the MTR-1 family is identified based on the presence of twelve transmembrane domains in the protein or corresponding nucleic acid molecule. As used herein, the term “transmembrane domain” refers to an amino acid sequence having at least about 10, preferably about 14, preferably about 18, more preferably about 19, and even more preferably about 20, 21 or 25 amino acid residues, of which at least about 60-70%, preferably about 80% and more preferably about 90% of the amino acid residues contain non-polar side chains, for example, alanine, valine, leucine, isoleucine, proline, phenylalanine, tryptophan, and methionine. A transmembrane domain is lipophilic in nature.

Preferred MTR-1 molecules of the present have an amino acid sequence sufficiently homologous, as defined herein, to the amino acid sequence of SEQ ID NO:35 or the amino acid sequence encoded by the nucleotide sequence of the DNA insert of the plasmid deposited with ATCC as Accession No: 98550.

As used interchangeably herein an “MTR-1 activity”, “biological activity of MTR-1” or “functional activity of MTR-1” refer to an activity exerted by an MTR-1 protein or nucleic acid molecule on an MTR-1 responsive cell as determined in vivo or in vitro, according to standard techniques. In one embodiment, an MTR-1 activity is a direct activity, such as transport of a metabolite. In another embodiment, an MTR-1 activity is an indirect activity, such as stimulating an enzyme within a cell by rate limiting transport of a metabolite into the cell. In a preferred embodiment, an MTR-1 activity is at least one or more of the following activities: (i) ability to be translocated into a plasma membrane of a cell; and (ii) transport of a metabolite across the plasma membrane. Preferably, an MTR-1 activity is at least one or more of the following activities: (i) uptake of a metabolite into a cell (ii) release of a metabolite from a cell; (iii) ability to regulate metabolite concentration within a cell.

As used herein the phrase “transport a metabolite” refers to the transport of a metabolite across the plasma membrane. The transport of a metabolite can occur, for example, by facilitated diffusion. Facilitated diffusion allows a metabolite to be transported down a concentration gradient either into or out of the cell. In one embodiment, the transporter protein can release a metabolite from within a cell. In another embodiment, the metabolite transporter is responsible for the uptake of a metabolite from the blood into the cytoplasm of a cell. The term “metabolite” as used herein, refers to a product of metabolism, for example, a carbon structure. In a preferred embodiment, the metabolite is a sugar, e.g., glucose.

Accordingly, another embodiment of the invention features isolated MTR-1 proteins having an MTR-1 activity. Preferred MTR-1 proteins have twelve transmembrane domains and an MTR-1 activity. In another preferred embodiment, the MTR-1 protein has twelve transmembrane domains, an MTR-1 activity and an amino acid sequence sufficiently homologous to an amino acid sequence of SEQ ID NO:35 or the amino acid sequence encoded by the nucleic acid sequence of the DNA insert of the plasmid deposited with ATCC as Accession No: 98550.

Another embodiment of the invention features MTR-1 molecules which contain a pentameric motif. The term “pentameric motif” as used herein, refers to a conserved motif of an MTR-1 protein member (or transmembrane segment transporter superfamily member) which includes five amino acid residues. A pentameric motif comprises the following amino acid sequence: R-X-G-R-X₁ (SEQ ID NO:37), wherein X is any amino acid residue and X₁ is either arginine (R) or lysine (K), and wherein the standard IUPAC one-letter codes for the amino acids are used. As used herein, all sequences containing one-letter codes for amino acids are defined by the IUPAC standards. A pentameric motif is substantially homologous between MTR-1 protein family members such that the motif is at least about 60%, even more preferably about 80% homologous to the pentameric motif amino acid sequence of SEQ ID NO:35.

Accordingly, in one embodiment, MTR-1 proteins of the invention contain twelve transmembrane domains and have an amino acid sequence sufficiently homologous to a pentameric motif amino acid sequence of SEQ ID NO:35. In another preferred embodiment, the MTR-1 protein has twelve transmembrane domains, an amino acid sequence sufficiently homologous to a pentameric motif amino acid sequence of SEQ ID NO:35 and an MTR-1 activity.

Yet another embodiment of the invention features MTR-1 proteins which contain at least one octameric motif. The term “octameric motif”, as used herein, refers to a conserved motif of an MTR-1 family member (or a transmembrane segment transporter superfamily member) which includes eight amino acid residues. An octameric motif comprises the following amino acid sequence: E-X-X-X-X-X-X-R (SEQ ID NO:38), wherein X is any amino acid residue. In one embodiment, an MTR-1 protein includes an octameric motif having at least about 25%, preferably at least 30%, and more preferably about 40% amino acid sequence homology to an octameric motif amino acid sequence of SEQ ID NO:35. Preferably, an MTR-1 protein includes at least two octameric motifs. For example, in one embodiment, an MTR-1 protein has two octameric motifs and has at least about 25%, more preferably about 30%, and even more preferably about 40% homology to the octameric motif amino acid sequence of SEQ ID NO:35.

In another embodiment of the invention, the MTR-1 protein contains at least one extended loop segment between the transmembrane domains. The term “extended loop segment” as used herein, refers to an extended segment of amino acid residues connecting two transmembrane domains. An extended segment of amino acids has about 40 to about 70, preferably about 50 to about 70, and more preferably about 60 to about 70 amino acid residues. In a preferred embodiment, the extended loop segment is an intracellular loop joining the sixth and seventh transmembrane segments of the MTR-1 protein. Another preferred embodiment features an extended loop segment sufficiently homologous to the amino acid sequence of SEQ ID NO:35 (amino acid residues 220-282).

Accordingly, another embodiment of the invention is an MTR-1 protein having twelve transmembrane domains, a pentameric motif, and at least one amino acid sequence sufficiently homologous to an octameric motif amino acid sequence of SEQ ID NO:35. In another embodiment, MTR-1 proteins have twelve transmembrane domains, a pentameric motif, and at least one extended loop segment connecting transmembrane domains. Even more preferably, the MTR-1 proteins also have MTR-1 activity.

In one embodiment, the MTR-1 nucleic acid and protein molecules of the present invention are murine MTR-1 molecules. A murine MTR-1 nucleic acid molecule was identified from a murine choroid plexus cDNA library (described in further detail in Example 10). The nucleotide sequence of the isolated murine MTR-1 cDNA and the predicted amino acid sequence of the murine MTR-1 protein are shown in FIG. 4 and in SEQ ID NOs:34 and 35, respectively. A plasmid containing the full length nucleotide sequence encoding murine MTR-1 was deposited with ATCC on Sep. 26, 1997 and assigned Accession Number 98550. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

A search of Dbest™ (an EST database) using mouse MTR-1 revealed one similar human amino acid sequence from human placenta (Accession No. R59842) which is described as a metabolite transporter and which is 81% identical to amino acids 325-622 of the murine MTR-1 amino acid sequence of SEQ ID NO:35.

3 kb and 4 kb MTR-1 mRNA transcripts are expressed in mouse tissues including heart, skeletal muscle and kidney. Chromosomal mapping indicates that the murine MTR-1 gene is located on chromosome 10 near marker D10MIT86. This region is syntenic to 6q in human. Chromosomal mapping of the partial human gene (Accession No. R59842) indicates that this gene is located on chromosome 6q. Three human diabetes genes, insulin dependent diabetes mellitus 5 and 8 and transient neonatal diabetes mellitus, were mapped close to, but not directly on, this region.

The murine MTR-1 gene (also referred to as “TANGO 49”), which is approximately 3689 nucleotides in length, encodes a protein having a molecular weight of approximately 67 kD and which is approximately 622 amino acid residues in length. The murine MTR-1 protein contains twelve transmembrane domains. An MTR-1 transmembrane domain can be found, for example, from about amino acids 44-67, 85-106, 112-130, 142-160, 170-192, 202-220, 283-304, 322-343, 351-368, 469-488, 503-523, and 534-552 of SEQ ID NO:35. An MTR-1 pentameric motif can be found, for example, from about amino acids 107-111 of SEQ ID NO:235, and MTR-1 octameric motifs can be found at least, for example, from about amino acids 161-168 and 494-501 of SEQ ID NO:35.

An alignment of the murine MTR-1 amino acid sequence to other members of the transmembrane segment transporter superfamily of proteins is presented in FIGS. 5 and 6. FIG. 5 depicts an alignment of the amino acid sequences of murine MTR-1 (corresponding to amino acids 495 to 622 of SEQ ID NO:35) and human Glut 3 (SEQ ID NO:39; Swiss Prot™ Accession No. P11169). FIG. 6 depicts an alignment of the amino acid sequence of murine MTR-1 (corresponding amino acids 7 to 617 of SEQ ID NO:35) and a beet sugar transporter (SEQ ID NO:40). A pentameric motif is designated by italicized characters. Octameric motifs are designated by underlined, italicized characters. A transmembrane domain is indicated by bold characters.

C. LSP-1 Nucleic Acid and Protein Molecules

In one embodiment, an LSP-1 family member is identified based on the presence of “an immunoglobulin (Ig)-like domain” and a “transmembrane domain”, as defined herein, in the protein or corresponding nucleic acid molecule. As used herein, the term “immunoglobulin (Ig)-like domain” refers to a protein domain having an amino acid sequence of at least about 50, preferably at least about 60, more preferably at least about 70 amino acid residues, and even more preferably at least about 80-90 amino acids of which at least about 30%, preferably at least about 40%, more preferably at least about 50, 60 or 70% of the amino acids are homologous to the amino acid sequence if an immunoglobulin domain. The homologous amino acids between an Ig-like domain and an Ig domain can be positioned across the entire Ig-like domain, referred to herein as a “full” Ig-like domain. Alternatively, the homologous amino acids between an Ig-like domain and an Ig domain can be concentrated in regions of high homology dispersed throughout the Ig-like domain, referred to as a “partial” Ig-like domain. In a preferred embodiment, an Ig-like domain is located in the N-terminal region of an LSP-1 protein. For example, in one embodiment, an LSP-1 protein contains an Ig-like domain containing about amino acids 46-128 of SEQ ID NO:44, wherein amino acids homologous to as Ig domain are concentrated between amino acids 46-78 and amino acids 109-128.

In one embodiment, an LSP-1 protein contains a transmembrane domain, as defined herein, containing about amino acids 192-213 of SEQ ID NO:44.

In another preferred embodiment, an LSP-1 family member is identified further based on the presence of an N-terminal signal sequence, as defined herein. For example, in one embodiment, an LSP-1 protein contains a signal sequence containing about amino acids 1-20 of SEQ ID NO:44.

Preferred LSP-1 molecules of the present have an amino acid sequence sufficiently homologous, as defined herein, to the amino acid sequence of SEQ ID NO:44.

As used interchangeably herein an “LSP-1 activity”, “biological activity of LSP-1” or “functional activity of LSP-1” refer to an activity exerted by an LSP-1 protein, polypeptide or nucleic acid molecule on an LSP-1 responsive cell as determined in vivo or in vitro, according to standard techniques. In one embodiment, an LSP-1 activity is a direct activity, such as an association with or an enzymatic activity on a second protein. In another embodiment, an LSP-1 activity is an indirect activity, such as a cellular signaling activity mediated by interaction of the LSP-1 protein with a second protein. In a preferred embodiment, an LSP-1 activity is at least one or more of the following activities: (i) interaction of an LSP-1 protein on the cell surface with a second non-LSP-1 protein molecule on the surface of the same cell; (ii) interaction of an LSP-1 protein on the cell surface with a second non-LSP-1 protein molecule on the surface of a different cell; (iii) complex formation between a soluble LSP-1 protein and a cognate ligand; (iv) complex formation between a membrane-bound LSP-1 protein and a cytokine; (v) interaction of an LSP-1 protein with an intracellular protein via a second protein on the cell surface. In yet another preferred embodiment, an LSP-1 activity is at least one or more of the following activities: (i) modulation of cellular signal transduction; (ii) regulation of a cell involved in an inflammatory response; (iii) homing of a cell having an LSP-1 protein on its cell surface from a first to a second anatomical location; and (iv) modulation of a cell involved in the immune response.

Accordingly, another embodiment of the invention features isolated LSP-1 proteins and polypeptides having an LSP-1 activity. Preferred LSP-1 proteins have at least an N-terminal partial immunoglobulin (Ig) domain, a C-terminal transmembrane domain, and an LSP-1 activity. In another preferred embodiment, the LSP-1 protein has at least at least an N-terminal partial immunoglobulin (Ig) domain, a C-terminal transmembrane domain, an LSP-1 activity, and an amino acid sequence sufficiently homologous to an amino acid sequence of SEQ ID NO:44.

In a particularly preferred embodiment, the LSP-1 protein and nucleic acid molecules of the present invention are human LSP-1 molecules. A human LSP-1 cDNA was identified by the Signal Peptide trAP methodology described herein (See Example 14). The nucleic acid sequence of a positive cDNA clone was used to search the GenBank™ EST database and multiple ESTs having greater than 95% nucleotide identity were found. Three clones containing published nucleotide sequences were purchased from Research Genetics (Huntsville, Ala.) as part of the IMAGE Consortium. The full sequence of the human clone was assembled as depicted in FIG. 8. A nucleotide sequence of the isolated human LSP-1 cDNA and the predicted amino acid sequence of the human LSP-1 protein are shown in FIG. 7 and in SEQ ID NOs:43 and 44, respectively. In addition, the nucleotide sequence corresponding to the coding region of the human LSP-1 cDNA (nucleotides 1332-2009) is represented as SEQ ID NO:45.

An approximately 1.5 kb LSP-1 mRNA transcript is expressed at very low levels in most human tissues tested. Significant expression of LSP-1 mRNA was detected only in peripheral blood leukocytes. Chromosomal mapping indicates that the human LSP-1 gene maps to chromosome 7q21-q22, at 111-112 cM.

The human LSP-1 cDNA, which is approximately 2462 nucleotides in length, encodes a protein which is approximately 226 amino acid residues in length. The human LSP-1 protein contains an N-terminal signal sequence, an N-terminal partial immunoglobulin (Ig) domain, and a C-terminal transmembrane domain. An LSP-1 N-terminal partial immunoglobulin (Ig) domain can be found at least, for example, from about amino acids 46-128 of SEQ ID NO:244. An LSP-1 C-terminal transmembrane domain can be found at least, for example, from about amino acids 192-213 of SEQ ID NO:44. The human LSP-1 protein is a membrane-bound protein which further contains a signal sequence at about amino acids 1-20 of SEQ ID NO:44. The prediction of such a signal peptide can be made, for example, utilizing the computer algorithm SIGNALP (Henrik, et al. (1997) Protein Engineering 10:1-6).

D. TAP-1Nucleic Acid and Protein Molecules

The carboxy-terminal domain of TAP-1 molecules has homology with the carboxy-terminal domain of human thrombopoietin (TPO). TPO has been identified as the ligand of the c-mpl cytokine receptor which upon activation of the receptor functions as a megakaryocyte lineage-specific factor. The N-terminal domain of human TPO shares homology to erythropoietin (EPO). Thus, TAP-1, TPO and EPO may comprise a family of structurally- and functionally related factors. For example, TAP-1 molecules of the present invention may influence cell proliferation or differentiation, for example, hematopoietic cell proliferation or differentiation, e.g., the maturation or differentiation of, megakaryocytes into platelets, or erythroid progenitor cells into erythrocytes.

In one embodiment, a member of the TAP-1 family is identified based on the presence of at least one “serine-proline-threonine rich” in the protein or corresponding nucleic acid molecule. As used herein, the term “serine-proline-threonine rich” refers to a protein domain of about 7, 10, 15, 20, 30, 40, 50, 60, 70, or 80 amino acids, preferably about 10 to 30 amino acids, and most preferably about 18-22 amino acids having at least about 15% serine, proline and/or threonine residues, more preferably about 20 amino acids having at least about 20% serine, proline and/or threonine residues, and even more preferably about 20 amino acids having at least about 30% serine, proline and/or threonine residues.

In one embodiment, a TAP-1protein includes a serine-proline-threonine rich domain having at least about 20%, preferably at least about 30%, and more preferably about 40% amino acid sequence homology to a TAP-1 serine-proline-threonine rich domain, such as the domain of SEQ ID NO:50 (e.g., amino acid residues 1-20 or 40-60 of SEQ ID NO:50).

Preferred TAP-1 molecules of the present have an amino acid sequence sufficiently homologous, as defined herein, to all or a portion of the amino acid sequence of SEQ ID NO:50, such as a serine-proline-threonine rich domain of the amino acid sequence of SEQ ID NO:50. In one embodiment, a TAP-1 protein contains a serine-proline-threonine rich domain and a TAP-1activity.

In one embodiment, a member of the TAP-1family is identified based on the presence of a unique carboxyl terminal domain. The terms “C-terminal unique domain” or “carboxy-terminal domain” as used herein, refer to a protein domain of a TAP-1 protein family member which includes at least one serine-proline-threonine-rich domain and shares structural similarity to a human TPO C-terminal domain. A C-terminal unique domain is sufficiently homologous between TAP-1 protein family members such that the domain is at least about 40%, preferably about 50%, more preferably about 60%, even more preferably about 70%, 80%, or 90% homologous. As defined herein, a C-terminal unique domain of a TAP-1 protein family member, however, is not sufficiently homologous to a C-terminal unique domain of a member of another protein family, such as a TPO protein family member.

As used interchangeably herein, a “TAP-1 activity”, “biological activity of TAP-1” or “functional activity of TAP-1” refer to an activity exerted by a TAP-1protein, polypeptide or nucleic acid molecule on a TAP-1 responsive cell as determined in vivo or in vitro, according to standard techniques. In one embodiment, a TAP-1activity is a direct activity, such as an association with, or an enzymatic activity, on a second protein, e.g., a cell-surface receptor. In another embodiment, a TAP-1 activity is an indirect activity, such as a cellular signaling activity mediated by interaction of the TAP-1protein with a second protein. In a preferred embodiment, a TAP-1 activity is at least one or more of the following activities: (i) interaction, e.g., binding to, a cell-surface receptor, e.g., a hematopoietic-cell surface receptor; (ii) modulation of, e.g., activation or inhibition of, a cell-surface receptor; (iii) modulation of cellular signal transduction. In yet another preferred embodiment, a TAP-1 activity is at least one or more of the following activities: (i) regulation of cellular proliferation; (ii) regulation of cellular differentiation; (iii) regulation of cell survival; (iv) modulation of a cell involved in the immune response (v) regulation of maturation and/or differentiation of a hematopoietic stem cell; (vi) modulation of megakaryocytopoiesis; (vii) modulation of thrombopoiesis; (viii) regulation of maturation and/or differentiation of a megakaryocyte into platelets; and (ix) regulation of maturation and/or differentiation of erythroid progenitor cells into erythrocytes.

Accordingly, another embodiment of the invention features isolated TAP-1proteins and polypeptides having a TAP-1 activity. Preferred TAP-1 proteins have at least one serine-proline-threonine rich domain and a TAP-1 activity. In another preferred embodiment, the TAP-1protein has at least one serine-proline-threonine rich domain, a TAP-1 activity and an amino acid sequence sufficiently homologous to an amino acid sequence of SEQ ID NO:50.

In a particularly preferred embodiment, the TAP-1 protein and nucleic acid molecules of the present invention are human TAP-1 molecules. A partial human TAP-1 cDNA, also referred to as TANGO-94, was identified by analysis of an EST database using mouse TPO sequence as a probe. A partial human clone (jthqb070d08) was obtained from a human prostate cDNA library and was subsequently fully sequenced. Clone jthqb070d08 was deposited with the American Type Culture Collection on Oct. 2, 1997 and has ATCC Accession Number 98554. This clone contains a nucleotide sequence of the isolated C-terminal domain of human TAP-1 cDNA (nucleotides 1-528 corresponding to the C-terminus and 3′ untranslated sequence) and the predicted amino acid sequence of the human TAP-1 protein (amino acids 1-86) are shown in FIG. 12 and in SEQ ID NOs:49 and 50, respectively. The amino acid sequences showed 32% identity to the C-terminal part of human TPO. The nucleotide sequence corresponding to the coding region of the human TAP-1 cDNA are nucleotides 1-258 of SEQ ID NO:49, while nucleotides 259-528 correspond to the 3′ untranslated region of the gene.

Using the jthqb070d08 cDNA as a probe, a 3 kb TAP-1 mRNA transcript was shown to be expressed in human fetal liver tissues. In addition to the 3 kb band, several bands were detected in the Northern blots which may indicate splice variants of TAP-1. Two other less intense bands of approximately 5 and 2 kb were detected in all tissues tested.

Human TAP-1 is a partial sequence which is approximately 528 nucleotides in length, encodes a protein which is approximately 86 amino acid residues in length. The human TAP-1 protein contains four serine-proline-threonine-rich domains. A TAP-1 serine-proline-threonine-rich domain can be found at least, for example, from about amino acids 1-20 of SEQ ID NO:50 (Gly1 to Gly20 of SEQ ID NO:50); from about amino acids 21-40 of SEQ ID NO:50 (Ile20 to Ala40 of SEQ ID NO:50); from about amino acids 41-60 of SEQ ID NO:50 (Val40 to Gly60 of SEQ ID NO:50); and from about amino acids 61-81 of SEQ ID NO:50 (Pro61 to Thr81 of SEQ ID NO:50 ). The human TAP-1 C-terminal domain appears to encode a secreted protein, e.g., a growth factor secreted protein.

The human TAP-1 amino acid sequence shares significant homology, about 32% homology, with the C-terminal region of TPO. An alignment of the human TAP-1 amino acid sequences to human TPO sequences is presented in FIG. 13. The figure depicts an alignment of the amino acid sequences of TAP-1 (corresponding to amino acids 15 to 75 of SEQ ID NO:50) and human TPO sequences (Swiss-Prot™ Accession Numbers P40225, 1401246, and 939627). Identical residues are indicated in the row between the TAP-1 and the TPO sequences by a single amino acid code; conserved amino acid residues are indicated as (+).

Using the jthqb070d08 cDNA as a probe, 8 clones from a human fetal liver library were isolated and submitted for sequencing. 3 out of the 8 clones contain an insert of approximately 3 kb.

E. PA-I Nucleic Acid and Protein Molecules

In one embodiment, a PA-I family member is identified based on the presence of at least one “cysteine-rich domain” in the protein or corresponding nucleic acid molecule. As used herein, the term “cysteine-rich domain” refers to a protein domain having an amino acid sequence of at least about 20, preferably at least about 30, more preferably at least about 35-40 amino acid residues, of which at least about 2, preferably at least about 3, more preferably at least about 4, 5 or 6 amino acids are the amino acid residue cysteine. Cysteine-rich domains having lengths of 45-50 or 60 amino acid residues and having up to 7, 8, 9 or 10 cysteine residues are also within the scope of this invention. In one embodiment, a PA-I protein includes a cysteine rich domain having at least about 20%, preferably at least about 30%, and more preferably about 40% amino acid sequence homology to a proliferin-related protein cysteine-rich domain, such as the cysteine-rich domain of SEQ ID NO:56 (e.g., amino acid residues 198-243 of murine proliferin-related protein, Swiss-Prot™ Accession No. P04769).

Preferred PA-I molecules of the invention have an amino acid sequence sufficiently homologous, as defined herein, to an amino acid sequence of SEQ ID NO:54. In one embodiment, the PA-I protein contains a cysteine-rich domain and a PA-I activity.

As used interchangeably herein a “PA-I activity”, “biological activity of PA-I” or “functional activity of PA-I” refer to an activity exerted by a PA-I protein, polypeptide or nucleic acid molecule on a PA-I responsive cell as determined in vivo or in vitro, according to standard techniques. In one embodiment, a PA-I activity is a direct activity, such as an association with or an enzymatic activity on a second protein. In another embodiment, a PA-I activity is an indirect activity, such as a cellular signaling activity mediated by interaction of the PA-I protein with a second protein. In a preferred embodiment, a PA-I activity is at least one or more of the following activities: (i) formation of a complex with a cell-surface protein(s) or a ligand, e.g., a lipid or carbohydrate; (ii) formation of a complex with a prolactin and/or growth hormone receptor. In yet another preferred embodiment, a PA-I activity is at least one or more of the following activities: (i) regulation of cellular growth; (ii) regulation of cellular proliferation; (iii) regulation of angiogenesis; (iv) regulation of cellular differentiation; and (v) regulation of cell survival.

Accordingly, another embodiment of the invention features isolated PA-I proteins and polypeptides having a PA-I activity. Preferred PA-I proteins have at least one cysteine-rich domain and a PA-I activity. In another preferred embodiment, the PA-I protein has at least one cysteine-rich domain, a PA-I activity and an amino acid sequence sufficiently homologous to an amino acid sequence of SEQ ID NO:54.

Yet another embodiment of the invention features PA-I molecules which contain a signal sequence, as defined herein. In a further embodiment, the invention features PA-I molecules which are secreted. As used herein, “secreted” refers to protein molecules which have the ability to be directed to the cellular plasma membrane (usually through a signal peptide) and subsequently released into the extracellular space. Such secreted PA-I molecules lack a transmembrane domain.

In a particularly preferred embodiment, the PA-I protein and nucleic acid molecules of the present invention are human PA-I molecules. Using clone aa014234 as a probe for northern blots, a band of 1 Kb was detected in human placenta tissue (see FIG. 16).

In another preferred embodiment, the PA-I protein and nucleic acid molecules of the present invention are murine PA-I molecules. A murine PA-I cDNA (also referred to as MOPAI or TANGO 95) was identified by homology with human growth hormone. In particular, a human growth hormone cDNA sequence was used to search the GenBank™ EST database, and clone aa014234 was identified. This clone, containing the published nucleotide sequence, was purchased from Research Genetics (Huntsville, Ala.) as part of the IMAGE Consortium and subsequently fully sequenced. A nucleotide sequence of the isolated murine PA-I cDNA and the predicted amino acid sequence of the murine PA-I protein are shown in FIG. 14 and in SEQ ID NOs:53 and 54, respectively. In addition, the nucleotide sequence corresponding to the coding region of the murine PA-I cDNA (nucleotides 55-816) is represented as SEQ ID NO:55.

A BlastP search (BLAST™ searching, utilizing an amino acid sequence against a protein database), using the translation product (frame 1) of the cDNA sequence represented as SEQ ID NO:53, revealed homology to proteins belonging to the prolactin-growth hormone superfamily. One example of such a protein is mouse proliferin-related protein, which is 243 amino acids in length and is 35% identical (see FIG. 15) to amino acids 1-247 of the murine PA-I amino acid sequence depicted in FIG. 14 and SEQ ID NO:54.

A 1 kb PA-I mRNA transcript is expressed in murine tissues from day 7 embryos (see FIG. 16).

The murine PA-I cDNA, which is 933 nucleotides in length, encodes a protein which is approximately 253 amino acid residues in length. The murine PA-I protein contains one cysteine-rich domain. A PA-I cysteine rich domain can be found at least, for example, from about amino acids 201-247 of SEQ ID NO:54 (Lys201 to Lys247 of the murine PA-I amino acid sequence). The murine PA-I protein is a secreted protein which lacks a transmembrane domain. The murine PA-I protein further contains a signal sequence at amino acids 1-30 of SEQ ID NO:54 (Met1 to Ser30 of the murine PA-I amino acid sequence). The prediction of such a signal peptide can be made utilizing the computer algorithm SIGNALP (Henrik, et al. (1997) Protein Engineering 10:1-6).

For conventience, the following table lists SEQ ID NOs for nucleic acid and polypeptide sequences of the invention:

| SEQ ID NO | Description of Sequence |
|---|---|
| 1 | human EDIRF I nucleotide sequence |
| 2 | human EDIRF I amino acid sequence |
| 3 | murine EDIRF I nucleotide sequence |
| 4 | murine EDIRF I amino acid sequence |
| 5 | human EDIRF II nucleotide sequence |
| 6 | human EDIRF II amino acid sequence |
| 7 | human EDIRF I coding region |
| 8 | murine EDIRF I coding region |
| 9 | human EDIRF II coding region |
| 34 | murine MTR-1 nucleotide sequence |
| 35 | murine MTR-1 amino acid sequence |
| 36 | murine MTR-1 coding region |
| 43 | human LSP-1 nucleotide sequence |
| 44 | human LSP-1 amino acid sequence |
| 45 | human LSP-1 coding region |
| 49 | human TAP-1 nucleotide sequence |
| 50 | human TAP-1 amino acid sequence |
| 51 | human TAP-1 coding region |
| 53 | murine PA-I (Proliferin analog I) nucleotide sequence |
| 54 | murine PA-I amino acid sequence |
| 55 | murine PA-I coding region |

various aspects of the invention are described in further detail in the following subsections:

I. Isolated Nucleic Acid Molecules

One aspect of the invention pertains to isolated nucleic acid molecules that encode EDIRF, MTR-1, LSP-1, TAP-1, and PA-I proteins or biologically active portions thereof, as well as nucleic acid fragments sufficient for use as hybridization probes to identify EDIRF, MTR-1, LSP-1, TAP-1, and PA-I -encoding nucleic acids (e.g., EDIRF, MTR-1, LSP-1, TAP-1, and PA-I mRNA) and fragments for use as PCR primers for the amplification or mutation of EDIRF, MTR-1, LSP-1, TAP-1, and PA-I nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated EDIRF, MTR-1, LSP-1, TAP-1, and PA-I nucleic acid molecule can contain less than about 5 kb, 4kb, 3kb, 2kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554, or a portion thereof, can be isolated using standard molecular biology techniques and the sequence information provided herein. Using all or portion of the nucleic acid sequence of SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554, as a hybridization probe, EDIRF, MTR-1, LSP-1, TAP-1, and PA-I nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 8, 9, 34, 36, 43, 45, 49, 51, 53, or 55, or the nucleotide sequence of the DNA insert of the plasmid deposited with ATCC as Accession Number 98613, 98614, 98550, or 98554 can be isolated by the polymerase chain reaction (PCR) using synthetic oligonucleotide primers designed based upon the sequence of SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to EDIRF, MTR-1, LSP-1, TAP-1, and PA-I nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to a human EDIRF cDNA. This cDNA comprises sequences encoding a human EDIRF protein (i.e., “the coding region”, from nucleotides 69-608), as well as 5′ untranslated sequences (nucleotides 1-68) and 3′ untranslated sequences (nucleotides 609-738). Alternatively, the nucleic acid molecule can comprise only the coding region of SEQ ID NO:1 (e.g., nucleotides 69-608, corresponding to nucleotides 1-540 of SEQ ID NO:7).

In another preferred embodiment, an isolated nucleic acid molecule of the invention comprises the nucleotide sequence shown in SEQ ID NO:3. The sequence of SEQ ID NO:3 corresponds to the murine EDIRF cDNA. This cDNA comprises sequences encoding the murine EDIRF protein (i.e., “the coding region”, from nucleotides 74-613), as well as 5′ untranslated sequences (nucleotides 1-73) and 3′ untranslated sequences (nucleotides 614-756). Alternatively, the nucleic acid molecule can comprise only the coding region of SEQ ID NO:3 (e.g., nucleotides 74-613, corresponding to nucleotides 1-540 of SEQ ID NO:8).

In another preferred embodiment, an isolated nucleic acid molecule of the invention comprises the nucleotide sequence shown in SEQ ID NO:5. The sequence of SEQ ID NO:5 corresponds to a second human EDIRF cDNA, referred to herein as long-form human EDIRF. This cDNA comprises sequences encoding a human EDIRF protein. In one embodiment, this cDNA comprises a coding region from nucleotides 129-911, as well as 5′ untranslated sequences from nucleotides 1-128 and 3′ untranslated sequences from nucleotides 912-1259. Alternatively, the nucleic acid molecule can comprise only the coding region of SEQ ID NO:5 (e.g., nucleotides 129-911, corresponding to nucleotides 1-783 of SEQ ID NO:9). The human EDIRF cDNA of SEQ ID NO:5 also contains additional in frame ATG codons at positions 264-266 and 309-311 of SEQ ID NO:5 encoding methionines at positions 46 and 61 of SEQ ID NO:6, respectively. Thus, in another embodiment, the nucleotide sequence of SEQ ID NO:5 comprises a coding region from nucleotides 264-911, as well as 5′ untranslated sequences from nucleotides 1-263 and 3′ untranslated sequences from nucleotides 912-1259. In yet another embodiment, the nucleotide sequence of SEQ ID NO:5 comprises a coding region from nucleotides 309-911, as well as 5′ untranslated sequences from nucleotides 1-308 and 3′ untranslated sequences from nucleotides 912-1259. The ATG codons of SEQ ID NO:6, at positions 1-3, 264-266, and 309-311 are in a less favorable context for translation than is the ATG codon at position 1-3 of SEQ ID NO:1, indicating that human EDIRF proteins may be translated with a lower efficiency from the long-form human EDIRF nucleotide sequence of SEQ ID NO:5 as compared to the human EDIRF nucleotide sequence of SEQ ID NO:1.

In another preferred embodiment, an isolated nucleic acid molecule of the invention comprises the nucleotide sequence shown in SEQ ID NO:34 or SEQ ID NO:36 or the nucleotide sequence of the DNA insert of the plasmid deposited with ATCC as Accession No: 98550. The sequence of SEQ ID NO:34 corresponds to the murine MTR-1 cDNA. This cDNA comprises sequences encoding the murine MTR-1 protein (i.e., “the coding region”, from nucleotides 57-1925), as well as 5′ untranslated sequences (nucleotides 1 to 56) and 3′ untranslated sequences (nucleotides 1925-3689). Alternatively, the nucleic acid molecule can comprise only the coding region of SEQ ID NO:34 (e.g., nucleotides 57-1925) as set forth in SEQ ID NO:36.

In another preferred embodiment, an isolated nucleic acid molecule of the invention comprises the nucleotide sequence shown in SEQ ID NO:43. The sequence of SEQ ID NO:43 corresponds to the human LSP-1 cDNA. This cDNA comprises sequences encoding the human LSP-1 protein (i.e., “the coding region”, from nucleotides 1332-2009), as well as 5′ untranslated sequences (nucleotides 1-1331) and 3′ untranslated sequences (nucleotides 2010-2462). Alternatively, the nucleic acid molecule can comprise only the coding region of SEQ ID NO:43 (e.g., nucleotides 1332-2009, corresponding to SEQ ID NO:45).

In yet another preferred embodiment, an isolated nucleic acid molecule of the invention comprises the nucleotide sequence shown in SEQ ID NO:49. The sequence of SEQ ID NO:49 corresponds to the human TAP-1 cDNA. This cDNA comprises sequences encoding the human TAP-1 protein (i.e., “the coding region”, from nucleotides 1-258 of SEQ ID NO:49, corresponding to SEQ ID NO:51), as well as 3′ untranslated sequences (nucleotides 259-528 of SEQ ID NO:49). Alternatively, the nucleic acid molecule can comprise only the coding region of SEQ ID NO:49 (e.g., nucleotides 1-258 of SEQ ID NO:49, corresponding to SEQ ID NO:5 1).

In still another preferred embodiment, an isolated nucleic acid molecule of the invention comprises the nucleotide sequence shown in SEQ ID NO:53. The sequence of SEQ ID NO:53 corresponds to the murine PA-I cDNA. This cDNA comprises sequences encoding the murine PA-I protein (i.e., “the coding region”, from nucleotides 55-816 of SEQ ID NO:53, corresponding to SEQ ID NO:55), as well as 5′ untranslated sequences (nucleotides 1-54 of SEQ ID NO:53) and 3′ untranslated sequences (nucleotides 817-933 of SEQ ID NO:53). Alternatively, the nucleic acid molecule can comprise only the coding region of SEQ ID NO:53 (e.g., nucleotides 55-816, corresponding to SEQ ID NO:55). In another preferred embodiment, an isolated nucleic acid molecule of the invention comprises the nucleotide sequence of the DNA insert of the plasmid deposited with ATCC as Accession Number 98613, 98614, 98550, or 98554.

In another preferred embodiment, an isolated nucleic acid molecule of the invention comprises a nucleic acid molecule which is a complement of the nucleotide sequence shown in SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554, or a portion of any of these nucleotide sequences. A nucleic acid molecule which is complementary to the nucleotide sequence shown in SEQ ID NO:1, 3, 5, 7, 8, 9, 34, 36, 43, 45, 49, 51, 53, or 55, or the nucleotide sequence of the DNA insert of the plasmid deposited with ATCC as Accession Number 98613, 98614, 98550, or 98554 is one which is sufficiently complementary to the nucleotide sequence shown in SEQ ID NO:1, 3, 5, 7, 8, 9, 34, 36, 43, 45, 49, 51, 53, or 55, or the nucleotide sequence of the DNA insert of the plasmid deposited with ATCC as Accession Number 98613, 98614, 98550, or 98554, such that it can hybridize to the nucleotide sequence shown in SEQ ID NO:1, 3, 5, 7, 8, 9, 34, 36, 43, 45, 49, 51, 53, or 55, or the nucleotide sequence of the DNA insert of the plasmid deposited with ATCC as Accession Number 98613, 98614, 98550, or 98554, thereby forming a stable duplex.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95% or more homologous to the nucleotide sequence shown in SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554, or a portion of any of these nucleotide sequences.

Moreover, the nucleic acid molecule of the invention can comprise only a portion of the nucleic acid sequence of SEQ ID NO:1, 3, 5, 7, 8, 9, 34, 36, 43, 45, 49, 51, 53, or 55, the nucleotide sequence of the DNA insert of the plasmid deposited with ATCC as Accession Number 98613, 98614, 98550, or 98554, for example a fragment which can be used as a probe or primer or a fragment encoding a biologically active portion of EDIRF, MTR-1, LSP-1, TAP-1, or PA-I. The nucleotide sequence determined from the cloning of the human and murine EDIRF, MTR-1, LSP-1, TAP-1, and PA-I genes allows for the generation of probes and primers designed for use in identifying and/or cloning EDIRF, MTR-1, LSP-1, TAP-1, and PA-I homologues in other cell types, e.g., from other tissues, as well as EDIRF, MTR-1, LSP-1, TAP-1, and PA-I homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO:1, 3, 5, 7, 8, 9, 34, 36, 43, 45, 49, 51, 53, or 55, the DNA insert of the plasmid deposited with ATCC as Accession Number 98613, 98614, 98550, or 98554 sense, or an anti-sense sequence of SEQ ID NO:1, 3, 5, 7, 8, 9, 34, 36, 43, 45, 49, 51, 53, or 55, the DNA insert of the plasmid deposited with ATCC as Accession Number 98613, 98614, 98550, or 98554, or of a naturally occurring mutant of SEQ ID NO:NO:1, 3, 5, 7, 8, 9, 34, 36, 43, 45, 49, 51, 53, or 55, the DNA insert of the plasmid deposited with ATCC as Accession Number 98613, 98614, 98550, or 98554.

Probes based on the human EDIRF nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an EDIRF, MTR-1, LSP-1, TAP-1, or PA-I protein, such as by measuring a level of an EDIRF, MTR-1, LSP-1, TAP-1, or PA-I -encoding nucleic acid in a sample of cells from a subject e.g., detecting EDIRF, MTR-1, LSP-1, TAP-1, or PA-I mRNA levels or determining whether a genomic EDIRF, MTR-1, LSP-1, TAP-1, or PA-I gene has been mutated or deleted.

A nucleic acid fragment encoding a “biologically active portion of EDIRF, MTR-1, LSP-1, TAP-1, or PA-I” can be prepared by isolating a portion of SEQ ID NO:NO:1, 3, 5, 7, 8, 9, 34, 36, 43, 45, 49, 51, 53, or 55, the DNA insert of the plasmid deposited with ATCC as Accession Number 98613, 98614, 98550, or 98554 which encodes a polypeptide having an EDIRF, MTR-1, LSP-1, TAP-1, or PA-I biological activity (the biological activities of the EDIRF, MTR-1, LSP-1, TAP-1, and PA-I proteins have previously been described), expressing the encoded portion of EDIRF, MTR-1, LSP-1, TAP-1, or PA-I protein (e.g., by recombinant expression in vitro) and assessing the activity of the encoded portion of EDIRF, MTR-1, LSP-1, TAP-1, or PA-I.

The invention further encompasses nucleic acid molecules that differ from the nucleotide sequence shown in SEQ ID NO:NOs:1, 3, 5, 7, 8, 9, 34, 36, 43, 45, 49, 51, 53, and 55 and from the nucleotide sequences of the DNA insert of the plasmid deposited with ATCC as Accession Number 98613, 98614, 98550, or 98554, or portions thereof, due to degeneracy of the genetic code and thus encode the same EDIRF, MTR-1, LSP-1, TAP-1, or PA-I protein as that encoded by the nucleotide sequence shown in SEQ ID NO:NO:1, 3, 5, 7, 8, 9, 34, 36, 43, 45, 49, 51, 53, or 55 or by the nucleotide sequence of the DNA insert of the plasmid deposited with ATCC as Accession Number 98613, 98614, 98550, or 98554. In another embodiment, an isolated nucleic acid molecule of the invention has a nucleotide sequence encoding a protein having an amino acid sequence shown in SEQ ID NO:2, 4, 6, 35, 44, 50, or 54.

In addition to the human and murine EDIRF, MTR-1, LSP-1, TAP-1, and PA-I nucleotide sequences shown in SEQ ID NO:NO:1, 3, 5, 7, 8, 9, 34, 36, 43, 45, 49, 51, 53, and 55, it will be appreciated by those skilled in the art that DNA sequence polymorphisms that lead to changes in the amino acid sequences of EDIRF, MTR-1, LSP-1, TAP-1, and PA-I may exist within a population (e.g., the human population). Such genetic polymorphism in the EDIRF, MTR-1, LSP-1, TAP-1, and PA-I genes may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. Allelic variants of any of these genes can be identified by sequencing the corresponding chromosomal portion at the indication location in multiple individuals.

As used herein, the terms “gene” and “recombinant gene” refer to nucleic acid molecules comprising an open reading frame encoding an EDIRF, MTR-1, LSP-1, TAP-1, or PA-I protein, preferably a mammalian EDIRF, MTR-1, LSP-1, TAP-1, or PA-I protein. Such natural allelic variations can typically result in 1-5% variance in the nucleotide sequence of the EDIRF, MTR-1, LSP-1, TAP-1, or PA-I gene. Any and all such nucleotide variations and resulting amino acid polymorphisms in EDIRF, MTR-1, LSP-1, TAP-1, or PA-I that are the result of natural allelic variation and that do not alter the functional activity of EDIRF, MTR-1, LSP-1, TAP-1, or PA-I are intended to be within the scope of the invention.

Moreover, nucleic acid molecules encoding EDIRF, MTR-1, LSP-1, TAP-1, or PA-I proteins from other species, and thus which have a nucleotide sequence which differs from the human or mouse sequences of SEQ ID NO:NO:1, 3, 5, 7, 8, 9, 34, 36, 43, 45, 49, 51, 53, and 55, or the nucleotide sequence of the DNA insert of the plasmid deposited with ATCC as Accession Number 98613, 98614, 98550, or 98554 are intended to be within the scope of the invention. Nucleic acid molecules corresponding to natural allelic variants and homologues of the EDIRF, MTR-1, LSP-1, TAP-1, or PA-I cDNA of the invention can be isolated based on their homology to the human or murine EDIRF, MTR-1, LSP-1, TAP-1, or PA-I nucleic acids disclosed herein using the human cDNA, murine cDNA, or portions thereof, as a hybridization probe according to standard hybridization techniques under stringent hybridization conditions. For example, a viral EDIRF, MTR-1, LSP-1, TAP-1, or PA-I cDNA can be isolated based on its homology to human or murine EDIRF, MTR-1, LSP-1, TAP-1, or PA-I. Nucleic acid or amino acid sequence homology can be determined by employing the techniques taught herein.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:NO:1, 3, 5, 7, 8, 9, 34, 36, 43, 45, 49, 51, 53, or 55 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the EDIRF, MTR-1, LSP-1, TAP-1, or PA-I sequence that may exist in the population, the skilled artisan will further appreciate that changes can be introduced by mutation into the nucleotide sequence of SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554, thereby leading to changes in the amino acid sequence of the encoded EDIRF, MTR-1, LSP-1, TAP-1, or PA-I protein, without altering the functional ability of the EDIRF, MTR-1, LSP-1, TAP-1, or PA-I protein. For example, nucleotide substitutions leading to amino acid substitutions at “non-essential” amino acid residues can be made in the sequence of SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554. A “non-essential” amino acid residue is a residue that can be altered from the wild-type sequence of EDIRF, MTR-1, LSP-1, TAP-1, or PA-I (e.g., the sequence of SEQ ID NO:2, 4, 6, 35, 44, 50, or 54) without altering the biological activity, whereas an “essential” amino acid residue is required for biological activity. For example, amino acid residues that are conserved among the EDIRF proteins of the present invention, are predicted to be particularly unamenable to alteration. Furthermore, amino acid residues that are conserved between EDIRF protein and other proteins having IL-17-like domains are not likely to be amenable to alteration.

In addition, the amino acid sequences of human EDIRF have at least one predicted N-glycosylation site corresponding to about amino acids 75-78 of SEQ ID NO:2 or to about amino acids 156-159 of SEQ ID NO:6 which is not likely to be amenable to alteration. Likewise, the amino acid sequence of murine EDIRF has at least one predicted N-glycosylation site corresponding to about amino acids 75-78 of SEQ ID NO:4 which is not likely to be amenable to alteration.

In another example, amino acid residues that are conserved among the MTR-1 protein of the present invention, as well as among other members of the transmembrane segment transporter superfamily (as indicated by the alignment and comparison of the amino acid sequence of SEQ ID NO:35 and the amino acid sequence of human-glucose transporter 3 as presented in FIG. 5, and beet sugar transporter as presented in FIG. 6) are predicted to be particularly unamenable to alteration. For example, transmembrane segment sugar transporters, as well as the MTR-1 protein of the present invention, contain a pentameric motif which is not likely to be amenable to alteration. Furthermore the conserved octameric motifs are not likely to be amenable to mutation. Other amino acid residues, however (e.g., those that are not conserved or only semi-conserved among members of the transmembrane segment transporter superfamily), may not be essential for activity and thus are likely to be amenable to alteration.

In another example, amino acid residues that are conserved among the LSP-1 proteins of the present invention are predicted to be particularly unamenable to alteration. Furthermore, amino acid residues that are conserved between LSP-1 protein and other proteins having Ig-like or Ig domains are not likely to be amenable to alteration. In addition, the amino acid sequence of human LSP-1 has at least one predicted N-glycosylation sites corresponding to about amino acids 100-103 of SEQ ID NO:44 which is not likely to be amenable to alteration.

In yet another example, amino acid residues of TAP-1 that are conserved among the family members of this invention (as indicated by an alignment and comparison of the amino acid sequences of SEQ ID NO:50 with sequences of TPO, e.g., human TPO shown in FIG. 13) are predicted to be essential in TAP-1 and thus are not likely to be amenable to alteration. Identical or conserved amino acid sequences between TAP-1 and human TPO are indicated in the middle row between these sequences in FIG. 13.

In still another example, amino acid residues of PA-I that are conserved among the prolactin-growth hormone family members of this invention (as indicated by the alignment and comparison of the amino acid sequences of SEQ ID NOs:54 and 56 presented as FIG. 15) are predicted to be essential in PA-I and thus are not likely to be amenable to alteration. For example, most proteins of the prolactin-growth hormone family, as well as the PA-I protein of the present invention, contain at least four cysteine residues among the cysteine rich domains (residues 101, 218, 235 and 244 of SEQ ID NO:54).

Accordingly, another aspect of the invention pertains to nucleic acid molecules encoding EDIRF proteins that contain changes in amino acid residues that are not essential for activity. Such EDIRF, MTR-1, LSP-1, TAP-1, or PA-I proteins differ in amino acid sequence from SEQ ID NO:2, 4, 6, 35, 44, 50, or 54 yet retain biological activity.

In one embodiment, the isolated nucleic acid molecule comprises a nucleotide sequence encoding a protein, wherein the protein comprises an amino acid sequence at least about 60% homologous to the amino acid sequence of SEQ ID NO:2, 4, 6, 35, 44, 50, or 54. Preferably, the protein encoded by the nucleic acid molecule is at least about 70% homologous to SEQ ID NO:2, 4, 6, 35, 44, 50, or 54, more preferably at least about 80% homologous to SEQ ID NO:2, 4, 6, 35, 44, 50, or 54, even more preferably at least about 90% homologous to SEQ ID NO:2, 4, 6, 35,44, 50, or 54, and most preferably at least about 95% homologous to SEQ ID NO:2, 4, 6, 35, 44, 50, or 54.

An isolated nucleic acid molecule encoding an EDIRF, MTR-1, LSP-1, TAP-1, or PA-I protein homologous to the protein of SEQ ID NO:2, 4, 6, 35, 44, 50, or 54 can be created by introducing one or more nucleotide substitutions, additions or deletions into the nucleotide sequence of SEQ ID NO:NO:1, 3, 5, 7, 8, 9, 34, 36, 43, 45, 49, 51, 53, or 55, or into the nucleotide sequence of the DNA insert of the plasmid deposited with ATCC as Accession Number 98613, 98614, 98550, or 98554, such that one or more amino acid substitutions, additions or deletions are introduced into the encoded protein. Mutations can be introduced into SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in EDIRF, MTR-1, LSP-1, TAP-1, or PA-I is preferably replaced with another amino acid residue from the same side chain family. Alternatively, in another embodiment, mutations can be introduced randomly along all or part of an EDIRF, MTR-1, LSP-1, TAP-1, or PA-I coding sequence, such as by saturation mutagenesis, and the resultant mutants can be screened for EDIRF, MTR-1, LSP-1, TAP-1, or PA-I biological activity to identify mutants that retain activity. Following mutagenesis of SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554, the encoded protein can be expressed recombinantly and the activity of the protein can be determined.

In one embodiment, a mutant polypeptide that is a variant of a polypeptide of the invention can be assayed for: (1) the ability to form protein:protein interactions with a polypeptide of the invention; (2) the ability to bind a ligand of a polypeptide of the invention; (3) the ability to bind with a modulator or substrate of a polypeptide of the invention; (4) the ability to modulate a physiological activity of a polypeptide of the invention, such as one of those disclosed herein; or (5) the ability to catalyze a reaction catalyzed by a polypeptide of the invention. In another preferred embodiment, a mutant EDIRF, MTR-1, LSP-1, TAP-1, or PA-I protein can be assayed for demonstration of the EDIRF, MTR-1, LSP-1, TAP-1, or PA-I activities, as described herein.

In addition to the nucleic acid molecules encoding EDIRF, MTR-1, LSP-1, TAP-1, and PA-I proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire EDIRF, MTR-1, LSP-1, TAP-1, or PA-I coding strand, or to only a portion thereof. In one embodiment, an antisense nucleic acid molecule is antisense to a “coding region” of the coding strand of a nucleotide sequence encoding EDIRF, MTR-1, LSP-1, TAP-1, or PA-I. The term “coding region” refers to the region of the nucleotide sequence comprising codons which are translated into amino acid residues (e.g., the coding region of human EDIRF corresponds to SEQ ID NO:7 or 9; the coding region of murine EDIRF corresponds to SEQ ID NO:8; the coding region of murine MTR-1 corresponds to SEQ ID NO:36; the coding region of human LSP-1 corresponds to SEQ ID NO:45; the coding region of human TAP-1 corresponds to SEQ ID NO:51; and the coding region of murine PAI-I corresponds to SEQ ID NO:55). In another embodiment, the antisense nucleic acid molecule is antisense to a “noncoding region” of the coding strand of a nucleotide sequence encoding EDIRF, MTR-1, LSP-1, TAP-1, or PA-I. The term “noncoding region” refers to 5′ and 3′ sequences which flank the coding region that are not translated into amino acids (i.e., also referred to as 5′ and 3′ untranslated regions).

Given the coding strand sequences encoding EDIRF, MTR-1, LSP-1, TAP-1, and PA-I disclosed herein (e.g., SEQ ID NO:7, SEQ ID NO:8 or SEQ ID NO:9), antisense nucleic acids of the invention can be designed according to the rules of Watson and Crick base pairing. The antisense nucleic acid molecule can be complementary to the entire coding region of EDIRF, MTR-1, LSP-1, TAP-1, or PA-I mRNA, but more preferably is an oligonucleotide which is antisense to only a portion of the coding or noncoding region of EDIRF, MTR-1, LSP-1, TAP-1, or PA-I mRNA. For example, the antisense oligonucleotide can be complementary to the region surrounding the translation start site of EDIRF, MTR-1, LSP-1, TAP-1, or PA-I mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an EDIRF, MTR-1, LSP-1, TAP-1, or PA-I protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave EDIRF, MTR-1, LSP-1, TAP-1, or PA-I mRNA transcripts to thereby inhibit translation of EDIRF, MTR-1, LSP-1, TAP-1, or PA-I mRNA. A ribozyme having specificity for an EDIRF, MTR-1, LSP-1, TAP-1, or PA-I -encoding nucleic acid can be designed based upon the nucleotide sequence of an EDIRF, MTR-1, LSP-1, TAP-1, or PA-I cDNA disclosed herein (i.e., SEQ ID NO:NO:1, 3, 5, 7, 8, 9, 34, 36, 43, 45, 49, 51, 53, or 55, or the DNA insert of the plasmid deposited with ATCC as Accession Number 98613, 98614, 98550, or 98554). For example, a derivative of a Tetrahymena L-19 IVS RNA can be constructed in which the nucleotide sequence of the active site is complementary to the nucleotide sequence to be cleaved in an EDIRF, MTR-1, LSP-1, TAP-1, or PA-I-encoding mRNA. See, e.g., Cech et al. U.S. Pat. No. 4,987,071; and Cech et al. U.S. Pat. No. 5,116,742. Alternatively, EDIRF, MTR-1, LSP-1, TAP-1, or PA-I mRNA can be used to select a catalytic RNA having a specific ribonuclease activity from a pool of RNA molecules. See, e.g., Bartel, D. and Szostak, J. W. (1993) Science 261:1411-1418.

Alternatively, EDIRF, MTR-1, LSP-1, TAP-1, or PA-I gene expression can be inhibited by targeting nucleotide sequences complementary to the regulatory region of the EDIRF, MTR-1, LSP-1, TAP-1, or PA-I (e.g., the EDIRF, MTR-1, LSP-1, TAP-1, or PA-I promoter and/or enhancers) to form triple helical structures that prevent transcription of the EDIRF, MTR-1, LSP-1, TAP-1, or PA-I gene in target cells. See generally, Helene, C. (1991) Anticancer Drug Des. 6(6):569-84; Helene, C. et al. (1992) Ann. N.Y Acad. Sci. 660:27-36; and Maher, L. J. (1992) Bioessays 14(12):807-15.

In preferred embodiments, the nucleic acids of EDIRF, MTR-1, LSP-1, TAP-1, or PA-I can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. USA 93: 14670-675.

PNAs of EDIRF, MTR-1, LSP-1, TAP-1, or PA-I can be used therapeutic and diagnostic applications. For example, PNAs can be used as antisense or antigene agents for sequence-specific modulation of gene expression by, e.g., inducing transcription or translation arrest or inhibiting replication. PNAs of EDIRF, MTR-1, LSP-1, TAP-1, or PA-I can also be used, e.g., in the analysis of single base pair mutations in a gene by, e.g., PNA directed PCR clamping; as 'artificial restriction enzymes when used in combination with other enzymes, e.g., S1 nucleases (Hyrup B. (1996) supra); or as probes or primers for DNA sequence and hybridization (Hyrup B. et al. (1996) supra; Perry-O'Keefe supra).

In another embodiment, PNAs of EDIRF, MTR-1, LSP-1, TAP-1, or PA-I can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of EDIRF, MTR-1, LSP-1, TAP-1, or PA-I can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Research 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988). In addition, oligonucleotides can be modified with hybridization-triggered cleavage agents (See, e.g., Krol et al., 1988, Bio Techniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated Proteins and Antibodies

One aspect of the invention pertains to isolated EDIRF, MTR-1, LSP-1, TAP-1, and PA-I proteins, and biologically active portions thereof, as well as polypeptide fragments suitable for use as immunogens to raise anti- EDIRF, MTR-1, LSP-1, TAP-1, and PA-I antibodies. In one embodiment, native EDIRF, MTR-1, LSP-1, TAP-1, and PA-I proteins can be isolated from cells or tissue sources by an appropriate purification scheme using standard protein purification techniques. In another embodiment, EDIRF, MTR-1, LSP-1, TAP-1, and PA-I proteins are produced by recombinant DNA techniques. Alternative to recombinant expression, an EDIRF, MTR-1, LSP-1, TAP-1, or PA-I protein or polypeptide can be synthesized chemically using standard peptide synthesis techniques.

An “isolated” or “purified” protein or biologically active portion thereof is substantially free of cellular material or other contaminating proteins from the cell or tissue source from which the EDIRF, MTR-1, LSP-1, TAP-1, or PA-I protein is derived, or substantially free from chemical precursors or other chemicals when chemically synthesized. The language “substantially free of cellular material” includes preparations of EDIRF, MTR-1, LSP-1, TAP-1, or PA-I protein in which the protein is separated from cellular components of the cells from which it is isolated or recombinantly produced. In one embodiment, the language “substantially free of cellular material” includes preparations of EDIRF, MTR-1, LSP-1, TAP-1, or PA-I protein having less than about 30% (by dry weight) of non-EDIRF, non-MTR-1, non-LSP-1, non-TAP-1, or non-PA-I protein (also referred to herein as a “contaminating protein”), more preferably less than about 20% of non-EDIRF, non-MTR-1, non-LSP-1, non-TAP-1, or non-PA-I protein, still more preferably less than about 10% of non-EDIRF, non-MTR-1, non-LSP-1, non-TAP-1, or non-PA-I protein, and most preferably less than about 5% non-EDIRF, non-MTR-1, non-LSP-1, non-TAP-1, or non-PA-I protein. When the EDIRF, MTR-1, LSP-1, TAP-1, or PA-I protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of EDIRF, MTR-1, LSP-1, TAP-1, or PA-I protein in which the protein is separated from chemical precursors or other chemicals which are involved in the synthesis of the protein. In one embodiment, the language “substantially free of chemical precursors or other chemicals” includes preparations of EDIRF, MTR-1, LSP-1, TAP-1, or PA-I protein having less than about 30% (by dry weight) of chemical precursors or non-EDIRF, non-MTR-1, non-LSP-1, non-TAP-1, or non-PA-I chemicals, more preferably less than about 20% chemical precursors or non-EDIRF, non-MTR-1, non-LSP-1, non-TAP-1, or non-PA-I chemicals, still more preferably less than about 10% chemical precursors or non-EDIRF, non-MTR-1, non-LSP-1, non-TAP-1, or non-PA-I chemicals, and most preferably less than about 5% chemical precursors or non-EDIRF, non-MTR-1, non-LSP-1, non-TAP-1, or non-PA-I chemicals.

Biologically active portions of an EDIRF, MTR-1, LSP-1, TAP-1, or PA-I protein include peptides comprising amino acid sequences sufficiently homologous to or derived from the amino acid sequence of the EDIRF, MTR-1, LSP-1, TAP-1, or PA-I protein, e.g., the amino acid sequence shown in SEQ ID NO:2, 4, 6, 35, 44, 50, or 54, which include fewer amino acids than the full length EDIRF, MTR-1, LSP-1, TAP-1, or PA-I proteins, and exhibit at least one activity of an EDIRF, MTR-1, LSP-1, TAP-1, or PA-I protein. Typically, biologically active portions comprise a domain or motif with at least one activity of the EDIRF, MTR-1, LSP-1, TAP-1, or PA-I protein. A biologically active portion of an EDIRF, MTR-1, LSP-1, TAP-1, or PA-I protein can be a polypeptide which is, for example, 10, 25, 50, 100 or more amino acids in length.

In one embodiment, a biologically active portion of an EDIRF protein comprises at least a C-terminal IL-17-like domain. In yet another embodiment, a biologically active portion of an EDIRF protein comprises at least a signal sequence. In an alternative embodiment, a biologically active portion of an EDIRF protein comprises an EDIRF amino acid sequence lacking a signal sequence.

It is to be understood that a preferred biologically active portion of an EDIRF protein of the present invention may contain at least one of the above-identified structural domains. A more preferred biologically active portion of an EDIRF protein may contain at least two of the above-identified structural domains. An even more preferred biologically active portion of an EDIRF protein may contain at least three of the above-identified structural domains.

In another embodiment, a biologically active portion of a LSP-1 protein comprises at least a transmembrane domain. In yet another embodiment, a biologically active portion of a LSP-1 protein comprises at least a signal sequence. In an alternative embodiment, a biologically active portion of a LSP-1 protein comprises a LSP-1 amino acid sequence lacking a transmembrane domain. In yet another embodiment, a biologically active portion of a LSP-1 protein comprises a LSP-1 amino acid sequence lacking a transmembrane domain and a signal sequence. Such a preferred LSP-1 molecules are referred to as a “LSP-1 extracellular domains”. For example, preferred LSP-1 extracellular domains contain at least about amino acids 1-190 of SEQ ID NO:2.

In yet another embodiment, a biologically active portion of a TAP-1 protein comprises at least one serine-proline-threonine rich region of TAP-1. In an alternative embodiment, a biologically active portion of a TAP-1 protein comprises at least a C-terminal unique domain of a TAP-1 protein.

In still another embodiment, a biologically active portion of a PA-I protein comprises at least one cysteine rich domain, characteristic of the prolactin-growth hormone superfamily of proteins. In another embodiment, a biologically active portion of a Proliferin analog I protein comprises at least a signal sequence.

Moreover, other biologically active portions, in which other regions of the protein are deleted, can be prepared by recombinant techniques and evaluated for one or more of the functional activities of a native EDIRF, MTR-1, LSP-1, TAP-1, or PA-I protein.

In a preferred embodiment, the EDIRF, MTR-1, LSP-1, TAP-1, or PA-I protein has an amino acid sequence shown in SEQ ID NO:2, 4, 6, 35, 44, 50, or 54. In other embodiments, the EDIRF, MTR-1, LSP-1, TAP-1, or PA-I protein is substantially homologous to SEQ ID NO:2, 4, 6, 35, 44, 50, or 54 and retains the functional activity of the protein of SEQ ID NO:2, 4, 6, 35, 44, 50, or 54 yet differs in amino acid sequence due to natural allelic variation or mutagenesis, as described in detail in subsection I above. Accordingly, in another embodiment, the EDIRF, MTR-1, LSP-1, TAP-1, or PA-I protein is a protein which comprises an amino acid sequence at least about 60% homologous to the amino acid sequence of SEQ ID NO:2, 4, 6, 35, 44, 50, or 54 and retains the functional activity of the EDIRF, MTR-1, LSP-1, TAP-1, or PA-I proteins of SEQ ID NO:2, 4, 6, 35, 44, 50, or 54, respectively. Preferably, the protein is at least about 70% homologous to SEQ ID NO:2, 4, 6, 35, 44, 50, or 54, more preferably at least about 80% homologous to SEQ ID NO:2, 4, 6, 35, 44, 50, or 54, even more preferably at least about 90% homologous to SEQ ID NO:2, 4, 6, 35, 44, 50, or 54, and most preferably at least about 95% or more homologous to SEQ ID NO:2, 4, 6, 35, 44, 50, or 54.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In one embodiment, an alignment is a global alignment, e.g., an overall sequence alignment. In another embodiment, an alignment is a local alignment. In a preferred embodiment, the length of a sequence aligned for comparison purposes is at least 30%, preferably at least 40%, more preferably at least 50%, even more preferably at least 60%, and even more preferably at least 70%, 80%, or 90% of the length of the reference sequence to which it is aligned (e.g., when aligning a second sequence to the EDIRF amino acid sequence of SEQ ID NO:2, at least 54, preferably at least 72, more preferably at least 90, even more preferably at least 108, and even more preferably at least 126, 144 or 162 amino acid residues are aligned; when aligning a second sequence to the MTR-1 amino acid sequence of SEQ ID NO:35 having 622 amino acid residues, at least 187, preferably at least 249, more preferably at least 311, even more preferably at least 373, and even more preferably at least 435, 498, or 560 are aligned; when aligning a second sequence to the LSP-1 amino acid sequence of SEQ ID NO:44 having 226 amino acid residues, at least 68, preferably at least 90, more preferably at least 113, even more preferably at least 136, and even more preferably at least 158, 181, or 203 are aligned; when aligning a second sequence to the TAP-1 amino acid sequence of SEQ ID NO:50 having 86 amino acid residues, at least 66, preferably at least 46, more preferably at least 26 are aligned; and when aligning a second sequence to the PA-I amino acid sequence of SEQ ID NO:54 having 253 amino acid residues, at least 76, preferably at least 101, more preferably at least 127, even more preferably at least 152, and even more preferably at least 177, 202, or 228 are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, 5, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to EDIRF, MTR-1, LSP-1, TAP-1, or PA-I nucleic acid molecules of the invention. BLAST protein searches can be performed with the XBLAST program, score=50, wordlength=3 to obtain amino acid sequences homologous to EDIRF, MTR-1, LSP-1, TAP-1, or PA-I protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides EDIRF, MTR-1, LSP-1, TAP-1, and PA-I chimeric or fusion proteins. As used herein, an EDIRF, MTR-1, LSP-1, TAP-1, or PA-I “chimeric protein” or “fusion protein” comprises an EDIRF, MTR-1, LSP-1, TAP-1, or PA-I polypeptide operatively linked to a non-EDIRF, non-MTR-1, non-LSP-1, non-TAP-1, or non-PA-I polypeptide, e.g., a heterologous polypeptide. An “EDIRF, MTR-1, LSP-1, TAP-1, or PA-I polypeptide” refers to a polypeptide having an amino acid sequence corresponding to EDIRF, MTR-1, LSP-1, TAP-1, or PA-I, whereas a “non-EDIRF, non-MTR-1, non-LSP-1, non-TAP-1, or non-PA-I polypeptide” refers to a polypeptide having an amino acid sequence corresponding to a protein which is not substantially homologous to the EDIRF, MTR-1, LSP-1, TAP-1, or PA-I protein, e.g., a protein which is different from the EDIRF, MTR-1, LSP-1, TAP-1, or PA-I protein and which is derived from the same or a different organism. Within an EDIRF, MTR-1, LSP-1, TAP-1, or PA-I fusion protein the EDIRF, MTR-1, LSP-1, TAP-1, or PA-I polypeptide can correspond to all or a portion of an EDIRF, MTR-1, LSP-1, TAP-1, or PA-I protein. In a preferred embodiment, an EDIRF, MTR-1, LSP-1, TAP-1, or PA-I fusion protein comprises at least one biologically active portion of an EDIRF, MTR-1, LSP-1, TAP-1, or PA-I protein. In another preferred embodiment, an EDIRF, MTR-1, LSP-1, TAP-1, or PA-I fusion protein comprises at least two biologically active portions of an EDIRF, MTR-1, LSP-1, TAP-1, or PA-I protein. In another preferred embodiment, an EDIRF, MTR-1, LSP-1, TAP-1, or PA-I fusion protein comprises at least three biologically active portions of an EDIRF, MTR-1, LSP-1, TAP-1, or PA-I protein. Within the fusion protein, the term “operatively linked” is intended to indicate that the EDIRF, MTR-1, LSP-1, TAP-1, or PA-I polypeptide and the non-EDIRF, non-MTR-1, non-LSP-1, non-TAP-1, or non-PA-I polypeptide are fused in-frame to each other. The non-EDIRF, non-MTR-1, non-LSP-1, non-TAP-1, or non-PA-I polypeptide can be fused to the N-terminus or C-terminus of the EDIRF, MTR-1, LSP-1, TAP-1, or PA-I polypeptide.

For example, in one embodiment, the fusion protein is a GST-EDIRF, GST-MTR-1, GST-LSP-1, GST-TAP-1, or GST-PA-I fusion protein in which the EDIRF, MTR-1, LSP-1, TAP-1, or PA-I sequences are fused to the C-terminus of the GST sequences. Such fusion proteins can facilitate the purification of recombinant EDIRF, MTR-1, LSP-1, TAP-1, or PA-I.

In another embodiment, the fusion protein is an EDIRF, MTR-1, LSP-1, TAP-1, or PA-I protein containing a heterologous signal sequence at its N-terminus. For example, the native EDIRF, LSP-1, or PA-I signal sequence (i.e., about amino acids 1 to 22 of SEQ ID NO:2, about amino acids 1 to 22 of SEQ ID NO:4, about amino acid residues 1-20 of SEQ ID NO:44) can be removed and replaced with a signal sequence from another protein. In certain host cells (e.g., mammalian host cells), expression and/or secretion of EDIRF, MTR-1, LSP-1, TAP-1, or PA-I can be increased through use of a heterologous signal sequence.

In yet another embodiment, the fusion protein is an EDIRF, MTR-1, LSP-1, TAP-1, or PA-I-immunoglobulin fusion protein in which the EDIRF, MTR-1, LSP-1, TAP-1, or PA-I sequences (e.g., those comprising primarily the extracellular domain) are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon, D. J. et al. (1989) Nature 337:525-531 and Capon U.S. Pat. Nos. 5,116,964 and 5,428,130 [CD4-IgG1 constructs]; Linsley, P. S. et al. (1991) J. Exp. Med. 173:721-730 [a CD28-IgG1 construct and a B7-1-IgG1 construct]; and Linsley, P. S. et al. (1991) J. Exp. Med. 174:561-569 and U.S. Pat. No. 5,434,131 [a CTLA4-IgG1]). Such fusion proteins have proven useful for modulating receptor-ligand interactions. Soluble derivatives of cell surface proteins of the tumor necrosis factor receptor (TNFR) superfamily proteins have been made consisting of an extracellular domain of the cell surface receptor fused to an immunoglobulin constant (Fc) region (see for example Moreland et al. (1997) N. Engl. J. Med. 337(3):141-147; van der Poll et al. (1997) Blood 89(10):3727-3734; and Ammann et al. (1997) J. Clin. Invest. 99(7):1699-1703.) Furthermore, fusion proteins have been made using the CH2 and CH3 domains of IgG fused downstream of murine IL-17 leader sequences and upstream of murine CTLA-8 sequences and upstream of HVS13 sequences (see for example Yao et al. (1995) Immunity 8:811-821.)

The EDIRF, MTR-1, LSP-1, TAP-1, or PA-I-immunoglobulin fusion proteins of the invention can be incorporated into pharmaceutical compositions and administered to a subject to inhibit an interaction between an EDIRF, MTR-1, LSP-1, TAP-1, or PA-I protein and an EDIRF, MTR-1, LSP-1, TAP-1, or PA-I receptor on the surface of a cell, to thereby suppress EDIRF, MTR-1, LSP-1, TAP-1, or PA-I-mediated cellular function in vivo. The EDIRF, MTR-1, LSP-1, TAP-1, or PA-I-immunoglobulin fusion proteins can be used to affect the bioavailability of an EDIRF, MTR-1, LSP-1, TAP-1, or PA-I protein. Inhibition of the EDIRF, MTR-1, LSP-1, TAP-1, or PA-I protein/EDIRF, MTR-1, LSP-1, TAP-1, or PA-I receptor interaction may be useful therapeutically, for example, in regulation of the cellular immune response, regulation of inflammation, or regulation of hematopoiesis. Moreover, the EDIRF, MTR-1, LSP-1, TAP-1, or PA-I-immunoglobulin fusion proteins of the invention can be used as immunogens to produce anti-EDIRF, MTR-1, LSP-1, TAP-1, or PA-I antibodies in a subject, to purify EDIRF, MTR-1, LSP-1, TAP-1, or PA-I receptors and in screening assays to identify molecules which inhibit the interaction of an EDIRF, MTR-1, LSP-1, TAP-1, or PA-I protein with an EDIRF, MTR-1, LSP-1, TAP-1, or PA-I receptor.

Preferably, an EDIRF, MTR-1, LSP-1, TAP-1, or PA-I chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An EDIRF, MTR-1, LSP-1, TAP-1, or PA-I -encoding nucleic acid can be cloned into such an expression vector such that the fusion moiety is linked in-frame to the EDIRF, MTR-1, LSP-1, TAP-1, or PA-I protein.

The present invention also pertains to variants of the EDIRF, MTR-1, LSP-1, TAP-1, or PA-I proteins which function as either EDIRF, MTR-1, LSP-1, TAP-1, or PA-I agonists (mimetics) or as EDIRF, MTR-1, LSP-1, TAP-1, or PA-I antagonists. Variants of the EDIRF, MTR-1, LSP-1, TAP-1, or PA-I protein can be generated by mutagenesis, e.g., discrete point mutation or truncation of the EDIRF, MTR-1, LSP-1, TAP-1, or PA-I protein. An agonist of the EDIRF, MTR-1, LSP-1, TAP-1, or PA-I protein can retain substantially the same, or a subset, of the biological activities of the naturally occurring form of the EDIRF, MTR-1, LSP-1, TAP-1, or PA-I protein. An antagonist of the EDIRF, MTR-1, LSP-1, TAP-1, or PA-I protein can inhibit one or more of the activities of the naturally occurring form of the EDIRF, MTR-1, LSP-1, TAP-1, or PA-I protein by, for example, competitively binding to an EDIRF, MTR-1, LSP-1, TAP-1, or PA-I receptor or an EDIRF, MTR-1, LSP-1, TAP-1, or PA-I-binding protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the EDIRF, MTR-1, LSP-1, TAP-1, or PA-I proteins.

In one embodiment, variants of the EDIRF, MTR-I, LSP-1, TAP-1, or PA-I protein which function as either EDIRF, MTR-1, LSP-1, TAP-1, or PA-I agonists (mimetics) or as EDIRF, MTR-1, LSP-1, TAP-1, or PA-I antagonists can be identified by screening combinatorial libraries of mutants, (e.g., truncation mutants) of the EDIRF, MTR-1, LSP-1, TAP-1, or PA-I protein for EDIRF, MTR-1, LSP-1, TAP-1, or PA-I protein agonist or antagonist activity. In one embodiment, a variegated library of EDIRF, MTR-1, LSP-1, TAP-1, or PA-I variants is generated by combinatorial mutagenesis at the nucleic acid level and is encoded by a variegated gene library. A variegated library of EDIRF, MTR-1, LSP-1, TAP-1, or PA-I variants can be produced by, for example, enzymatically ligating a mixture of synthetic oligonucleotides into gene sequences such that a degenerate set of potential EDIRF, MTR-1, LSP-1, TAP-1, or PA-I sequences is expressible as individual polypeptides, or alternatively, as a set of larger fusion proteins (e.g., for phage display) containing the set of EDIRF, MTR-1, LSP-1, TAP-1, or PA-I sequences therein. There are a variety of methods which can be used to produce libraries of potential EDIRF, MTR-1, LSP-1, TAP-1, or PA-I variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential EDIRF, MTR-1, LSP-1, TAP-1, or PA-I sequences. Methods for synthesizing degenerate oligonucleotides are known in the art (see, e.g., Narang, S. A. (1983) Tetrahedron 39:3; Itakura et a. (1984) Annu. Rev. Biochem. 53:323; Itakura et al. (1984) Science 198:1056; Ike et al. (1983) Nucleic Acid Res. 11:477.

In addition, libraries of fragments of the EDIRF, MTR-1, LSP-1, TAP-1, or PA-I protein coding sequence can be used to generate a variegated population of EDIRF, MTR-1, LSP-1, TAP-1, or PA-I fragments for screening and subsequent selection of variants of an EDIRF, MTR-1, LSP-1, TAP-1, or PA-I protein. In one embodiment, a library of coding sequence fragments can be generated by treating a double stranded PCR fragment of an EDIRF, MTR-1, LSP-1, TAP-1, or PA-I coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the EDIRF, MTR-1, LSP-1, TAP-1, or PA-I protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of EDIRF, MTR-1, LSP-1, TAP-1, or PA-I proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify EDIRF, MTR-1, LSP-1, TAP-1, or PA-I variants (Arkin and Youvan (1992) Proc. Natl. Acad. Sci. USA 89:7811-7815; Delagrave et al. (1 993) Protein Engineering 6(3):327-33 1).

In one embodiment, cell based assays can be exploited to analyze a variegated EDIRF, MTR-1, LSP-1, TAP-1, or PA-I library. For example, a library of expression vectors can be transfected into a cell line which ordinarily secretes EDIRF, MTR-1, LSP-1, TAP-1, or PA-I protein. Supernatants from the transfected cells are then contacted with EDIRF, MTR-1, LSP-1, TAP-1, or PA-I -responsive cells and the effect of the mutation in EDIRF, MTR-1, LSP-1, TAP-1, or PA-I can be detected, e.g., by measuring any of a number of EDIRF, MTR-1, LSP-1, TAP-1, or PA-I-responsive cell responses. Plasmid DNA can then be recovered from the mutant EDIRF, MTR-1, LSP-1, TAP-1, or PA-I-secreting cells which score for inhibition, or alternatively, potentiation of the EDIRF, MTR-1, LSP-1, TAP-1, or PA-I-dependent response, and the individual clones further characterized.

An isolated EDIRF, MTR-1, LSP-1, TAP-1, or PA-I protein, or a portion or fragment thereof, can be used as an immunogen to generate antibodies that bind EDIRF, MTR-1, LSP-1, TAP-1, or PA-I using standard techniques for polyclonal and monoclonal antibody preparation. The full-length EDIRF, MTR-1, LSP-1, TAP-1, or PA-I protein can be used or, alternatively, the invention provides antigenic peptide fragments of EDIRF, MTR-1, LSP-1, TAP-1, or PA-I for use as immunogens. The antigenic peptide of EDIRF, MTR-1, LSP-1, TAP-1, or PA-I comprises at least 8 amino acid residues of the amino acid sequence shown in SEQ ID NO:2, 4, 6, 35, 44, 50, or 54 and encompasses an epitope of EDIRF, MTR-1, LSP-1, TAP-1, or PA-I such that an antibody raised against the peptide forms a specific immune complex with EDIRF, MTR-1, LSP-1, TAP-1, or PA-I. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of EDIRF, MTR-1, LSP-1, TAP-1, or PA-I that are located on the surface of the protein, e.g., hydrophilic regions.

A EDIRF, MTR-1, LSP-1, TAP-1, or PA-I immunogen typically is used to prepare antibodies by immunizing a suitable subject, (e.g., rabbit, goat, mouse or other mammal) with the immunogen. An appropriate immunogenic preparation can contain, for example, recombinantly expressed EDIRF, MTR-1, LSP-1, TAP-1, or PA-I protein or a chemically synthesized EDIRF, MTR-1, LSP-1, TAP-1, or PA-I polypeptide. The preparation can further include an adjuvant, such as Freund's complete or incomplete adjuvant, or similar immunostimulatory agent. Immunization of a suitable subject with an immunogenic EDIRF, MTR-1, LSP-1, TAP-1, or PA-I preparation induces a polyclonal anti-EDIRF, MTR-1, LSP-1, TAP-1, or PA-I antibody response.

Accordingly, another aspect of the invention pertains to anti-EDIRF, MTR-1, LSP-1, TAP-1, or PA-I antibodies. The term “antibody” as used herein refers to immunoglobulin molecules and immunologically active portions of immunoglobulin molecules, i.e., molecules that contain an antigen binding site which specifically binds (immunoreacts with) an antigen, such as EDIRF, MTR-1, LSP-1, TAP-1, or PA-I. Examples of immunologically active portions of immunoglobulin molecules include F(ab) and F(ab′)₂ fragments which can be generated by treating the antibody with an enzyme such as pepsin or papain, respectively. The invention provides polyclonal and monoclonal antibodies that bind EDIRF, MTR-1, LSP-1, TAP-1, or PA-I. The term “monoclonal antibody” or “monoclonal antibody composition”, as used herein, refers to a population of antibody molecules that contain only one species of an antigen binding site capable of immunoreacting with a particular epitope of EDIRF, MTR-1, LSP-1, TAP-1, or PA-I. A monoclonal antibody composition thus typically displays a single binding affinity for a particular EDIRF, MTR-1, LSP-1, TAP-1, or PA-I protein with which it immunoreacts.

Polyclonal anti-EDIRF, MTR-1, LSP-1, TAP-1, or PA-I antibodies can be prepared as described above by immunizing a suitable subject with an EDIRF, MTR-1, LSP-1, TAP-1, or PA-I immunogen. The anti-EDIRF, MTR-1, LSP-1, TAP-1, or PA-I antibody titer in the immunized subject can be monitored over time by standard techniques, such as with an enzyme linked immunosorbent assay (ELISA) using immobilized EDIRF, MTR-1, LSP-1, TAP-1, or PA-I. If desired, the antibody molecules directed against EDIRF, MTR-1, LSP-1, TAP-1, or PA-I can be isolated from the mammal (e.g., from the blood) and further purified by well known techniques, such as protein A chromatography to obtain the IgG fraction. At an appropriate time after immunization, e.g., when the anti-EDIRF, MTR-1, LSP-1, TAP-1, or PA-I antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an EDIRF, MTR-1, LSP-1, TAP-1, or PA-I immunogen as described above, and the culture supernatants of the resulting hybridoma cells are screened to identify a hybridoma producing a monoclonal antibody that binds EDIRF, MTR-1, LSP-1, TAP-1, or PA-I.

Any of the many well known protocols used for fusing lymphocytes and immortalized cell lines can be applied for the purpose of generating an anti-EDIRF, MTR-1, LSP-1, TAP-1, or PA-I monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind EDIRF, MTR-1, LSP-1, TAP-1, or PA-I, e.g., using a standard ELISA assay.

Alternative to preparing monoclonal antibody-secreting hybridomas, a monoclonal anti-EDIRF, MTR-1, LSP-1, TAP-1, or PA-I antibody can be identified and isolated by screening a recombinant combinatorial immunoglobulin library (e.g., an antibody phage display library) with EDIRF, MTR-1, LSP-1, TAP-1, or PA-I to thereby isolate immunoglobulin library members that bind EDIRF, MTR-1, LSP-1, TAP-1, or PA-I. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-EDIRF, MTR-1, LSP-1, TAP-1, or PA-I antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar ((1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jaspers et al. (1994) Biotechnology (NY) 12:899-903).

An anti-EDIRF, MTR-1, LSP-1, TAP-1, or PA-I antibody (e.g., monoclonal antibody) can be used to isolate EDIRF, MTR-1, LSP-1, TAP-1, or PA-I by standard techniques, such as affinity chromatography or immunoprecipitation. An anti-EDIRF, MTR-1, LSP-1, TAP-1, or PA-I antibody can facilitate the purification of natural EDIRF, MTR-1, LSP-1, TAP-1, or PA-I from cells and of recombinantly produced EDIRF expressed in host cells. Moreover, an anti-EDIRF, MTR-1, LSP-1, TAP-1, or PA-I antibody can be used to detect EDIRF, MTR-1, LSP-1, TAP-1, or PA-I protein (e.g., in a cellular lysate or cell supernatant) in order to evaluate the abundance and pattern of expression of the EDIRF, MTR-1, LSP-1, TAP-1, or PA-I protein. Anti-EDIRF, MTR-1, LSP-1, TAP-1, or PA-I antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine).

Conjugated antibodies of the invention can be used for modifying a given biological response, the drug moiety not being limited to classical chemical therapeutic agents. For example, the drug moiety can be a protein or polypeptide possessing a desired biological activity. Such proteins include, for example, toxins such as abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Arnon et al. (1985) “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al. (1987) “Antibodies For Drug Delivery ”, in Controlled Drug Delivery, 2nd ed., Robinson et al., Eds., Marcel Dekker, Inc., pp. 623-653; Thorpe, 1985 “Antibody Carriers Of Cytotoxic Agents In Cancer Therapy: A Review”, in Monoclonal Antibodies '84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al. (1982) Immunol. Rev. 62:119-158). Alternatively, an antibody can be conjugated to a second antibody to form an antibody heteroconjugate as described by Segal in U.S. Pat. No. 4,676,980.

III. Recombinant Expression Vectors and Host Cells

Another aspect of the invention pertains to vectors, preferably expression vectors, containing a nucleic acid encoding EDIRF, MTR-1, LSP-1, TAP-1, or PA-I (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., EDIRF, MTR-1, LSP-1, TAP-1, or PA-I proteins, mutant forms of EDIRF, MTR-1, LSP-1, TAP-1, or PA-I, fusion proteins, etc.).

The recombinant expression vectors of the invention can be designed for expression of EDIRF, MTR-1, LSP-1, TAP-1, or PA-I in prokaryotic or eukaryotic cells. For example, EDIRF, MTR-1, LSP-1, TAP-1, or PA-I can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in EDIRF, MTR-1, LSP-1, TAP-1, or PA-I activity assays, in EDIRF, MTR-1, LSP-1, TAP-1, or PA-I ligand binding (e.g., direct assays or competitive assays described in detail below), to generate antibodies specific for EDIRF, MTR-1, LSP-1, TAP-1, or PA-I proteins, as examples. In a preferred embodiment, an EDIRF, MTR-1, LSP-1, TAP-1, or PA-I fusion expressed in a retroviral expression vector of the present invention can be utilized to infect bone marrow cells which are subsequently transplanted into irradiated recipients. The pathology of the subject recipient is then examined after sufficient time has passed (e.g. six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS 174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the EDIRF, MTR-1, LSP-1, TAP-1, or PA-I expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, EDIRF, MTR-1, LSP-1, TAP-1, or PA-I can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to EDIRF, MTR-1, LSP-1, TAP-1, or PA-I mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1)1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, EDIRF, MTR-1, LSP-1, TAP-1, or PA-I protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding EDIRF, MTR-1, LSP-1, TAP-1, or PA-I or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) EDIRF, MTR-1, LSP-1, TAP-1, or PA-I protein. Accordingly, the invention further provides methods for producing EDIRF, MTR-1, LSP-1, TAP-1, or PA-I protein using the host cells of the invention. In one embodiment, the method comprises culturing the host cell of invention (into which a recombinant expression vector encoding EDIRF, MTR-1, LSP-1, TAP-1, or PA-I has been introduced) in a suitable medium such that EDIRF, MTR-1, LSP-1, TAP-1, or PA-I protein is produced. In another embodiment, the method further comprises isolating EDIRF, MTR-1, LSP-1, TAP-1, or PA-I from the medium or the host cell.

The host cells of the invention can also be used to produce nonhuman transgenic animals. For example, in one embodiment, a host cell of the invention is a fertilized oocyte or an embryonic stem cell into which EDIRF, MTR-1, LSP-1, TAP-1, or PA-I-coding sequences have been introduced. Such host cells can then be used to create non-human transgenic animals in which exogenous EDIRF, MTR-1, LSP-1, TAP-1, or PA-I sequences have been introduced into their genome or homologous recombinant animals in which endogenous EDIRF, MTR-1, LSP-1, TAP-1, or PA-I sequences have been altered. Such animals are useful for studying the function and/or activity of EDIRF, MTR-1, LSP-1, TAP-1, or PA-I and for identifying and/or evaluating modulators of EDIRF, MTR-1, LSP-1, TAP-1, or PA-I activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous EDIRF, MTR-1, LSP-1, TAP-1, or PA-I gene has been altered by homologous recombination between the endogenous gene and an exogenous DNA molecule introduced into a cell of the animal, e.g., an embryonic cell of the animal, prior to development of the animal.

A transgenic animal of the invention can be created by introducing EDIRF, MTR-1, LSP-1, TAP-1, or PA-I-encoding nucleic acid into the male pronuclei of a fertilized oocyte, e.g., by microinjection, retroviral infection, and allowing the oocyte to develop in a pseudopregnant female foster animal. The EDIRF, MTR-1, LSP-1, TAP-1, or PA-I cDNA sequences of SEQ ID NO:1, 3, 5, 34, 43, 49, or 53, or the DNA insert of the plasmid deposited with ATCC as Accession Number 98613, 98614, 98550, or 98554 can be introduced as a transgene into the genome of a non-human animal. Intronic sequences and polyadenylation signals can also be included in the transgene to increase the efficiency of expression of the transgene. A tissue-specific regulatory sequence(s) can be operably linked to the EDIRF, MTR-1, LSP-1, TAP-1, or PA-I transgene to direct expression of EDIRF, MTR-1, LSP-1, TAP-1, or PA-I protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the EDIRF, MTR-1, LSP-1, TAP-1, or PA-I transgene in its genome and/or expression of EDIRF, MTR-1, LSP-1, TAP-1, or PA-I mRNA in tissues or cells of the animals. A transgenic founder animal can then be used to breed additional animals carrying the transgene. Moreover, transgenic animals carrying a transgene encoding EDIRF, MTR-1, LSP-1, TAP-1, or PA-I can further be bred to other transgenic animals carrying other transgenes.

To create a homologous recombinant animal, a vector is prepared which contains at least a portion of an EDIRF, MTR-1, LSP-1, TAP-1, or PA-I gene into which a deletion, addition or substitution has been introduced to thereby alter, e.g., functionally disrupt, the EDIRF, MTR-1, LSP-1, TAP-1, or PA-I gene. The EDIRF, MTR-1, LSP-1, TAP-1, or PA-I gene can be a human gene (e.g., the cDNA of SEQ ID NO:1, 5, 43, or 49), but more preferably, is a non-human homologue of a human EDIRF, MTR-1, LSP-1, TAP-1, or PA-I gene (e.g., the cDNA of SEQ ID NO:3, 34, or 53). For example, a mouse EDIRF, MTR-1, LSP-1, TAP-1, or PA-I gene of SEQ ID NO:3 can be used to construct a homologous recombination vector suitable for altering an endogenous EDIRF, MTR-1, LSP-1, TAP-1, or PA-I gene in the mouse genome. In a preferred embodiment, the vector is designed such that, upon homologous recombination, the endogenous EDIRF, MTR-1, LSP-1, TAP-1, or PA-I gene is functionally disrupted (i.e., no longer encodes a functional protein; also referred to as a “knock out” vector). Alternatively, the vector can be designed such that, upon homologous recombination, the endogenous EDIRF, MTR-1, LSP-1, TAP-1, or PA-I gene is mutated or otherwise altered but still encodes functional protein (e.g., the upstream regulatory region can be altered to thereby alter the expression of the endogenous EDIRF, MTR-1, LSP-1, TAP-1, or PA-I protein). In the homologous recombination vector, the altered portion of the EDIRF, MTR-1, LSP-1, TAP-1, or PA-I gene is flanked at its 5′ and 3′ ends by additional nucleic acid of the EDIRF, MTR-1, LSP-1, TAP-1, or PA-I gene to allow for homologous recombination to occur between the exogenous EDIRF gene carried by the vector and an endogenous EDIRF gene in an embryonic stem cell. The additional flanking EDIRF, MTR-1, LSP-1, TAP-1, or PA-I nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced EDIRF, MTR-1, LSP-1, TAP-1, or PA-I gene has homologously recombined with the endogenous EDIRF, MTR-1, LSP-1, TAP-1, or PA-I gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The EDIRF, MTR-1, LSP-1, TAP-1, or PA-I nucleic acid molecules, EDIRF, MTR-1, LSP-1, TAP-1, or PA-I proteins, and anti- EDIRF, MTR-1, LSP-1, TAP-1, and PA-I antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). For antibodies, examples of dosages are from about 0.1 milligram per kilogram to 100 milligrams per kilogram of body weight (generally 10 milligrams per kilogram to 20 milligrams per kilogram). If the antibody is to act in the brain, a dosage of 50 milligrams per kilogram to 100 milligrams per kilogram is usually appropriate. It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an EDIRF, MTR-1, LSP-1, TAP-1, or PA-I protein or anti-EDIRF, MTR-1, LSP-1, TAP-1, or PA-I antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials); and d) methods of treatment (e.g., therapeutic and prophylactic methods as well as such methods in the context of pharmacogenetics).

As described herein, an EDIRF protein of the invention has one or more of the following activities: (i) activation of an EDIRF-dependent signal transduction pathway; (ii) modulation of secretion of a non-IL-17 cytokine; (i) activation of an EDIRF-dependent signal transduction pathway; (ii) modulation of secretion of a non-IL-17 cytokine; (iii) interaction of an EDIRF protein with a non-EDIRF cell surface molecule (e.g., a proteoglycan or sulfated proteoglycan); (iv) interaction of an EDIRF protein with heparin; (v) modulation of secretion of IL-17; (vi) modulation of surface expression of a cellular adhesion molecule; (vii) modulation of a proinflammatory cytokine; (viii) modulation of a hematopoietic cytokine; (ix) modulation of the development or differentiation of an EDIRF-expressing cell; (x) modulation of the development or differentiation of a non-EDIRF-expressing cell; (xi) modulation of the homeostasis of an EDIRF-expressing cell; and (xii) modulation of the homeostasis of a non-EDIRF-expressing cell and can thus be used in, for example, (1) regulation of the cellular immune response; (2) regulation of inflammation; (3) regulation of hematopoiesis, or (4) control of differentiation or development, either in vitro or in vivo.

As described herein, a MTR-1 protein of the invention has the following activities: 1) ability to be translocated into the plasma membrane; 2) the uptake and/or release of metabolites from a cell and can thus be used as an transport agent for metabolites or as an indicator of inhibition or stimulation of metabolite transport by various compounds, either in vitro or in vivo.

As described herein, a LSP-1 protein of the invention has one or more of the following activities: (i) interaction of a LSP-1 protein on the cell surface with a second non-LSP-1 protein molecule on the surface of the same cell; (ii) interaction of a LSP-1 protein on the cell surface with a second non-LSP-1 protein molecule on the surface of a different cell; (iii) complex formation between a soluble LSP-1 protein and a cognate ligand; (iv) complex formation between a membrane-bound LSP-1 protein and a cytokine; (v) interaction of a LSP-1 protein with an intracellular protein via a second protein on the cell surface and can thus be used for (i) modulation of cellular signal transduction; (ii) regulation of a cell involved in an inflammatory response; (iii) homing of a cell having a LSP-1 protein on its cell surface from a first to a second anatomical location; and (iv) modulation of a cell involved in the immune response, either in vitro or in vivo.

As described herein, a TAP-1 protein of the invention has the following activities: (i) interaction, e.g., binding to, a cell-surface receptor, e.g., a hematopoietic-cell surface receptor; (ii) modulation of, e.g., activation or inhibition of, a cell-surface receptor (iii) modulation of cellular signal transduction and can thus be used to (i) regulate cellular proliferation; (ii) regulate cellular differentiation; (iii) regulate cell survival; (iv) modulate a cell involved in the immune response (v) regulate maturation and/or differentiation of a hematopoietic stem cell; (vi) modulate megakaryocytopoiesis; (vii) modulate thrombopoiesis; (viii) regulate maturation and/or differentiation of a megakaryocyte into platelets; and (ix) regulate maturation and/or differentiation of erythroid progenitor cells into erythrocytes, either in vitro or in vivo.

As described herein, a PA-I protein of the invention has one or more of the following activities: (i) formation of a complex with a cell-surface protein(s) or a ligand, e.g., a lipid or carbohydrate; (ii) formation of a complex with a prolactin and/or growth hormone receptor; (iii) regulation of cellular growth; (iv) regulation of cellular proliferation; (v) regulation of angiogenesis; (vi) regulation of cellular differentiation; and (vii) regulation of cell survival, and can thus be used to (i) modulate complex formation with a cell-surface protein(s) or a ligand, e.g., a lipid or carbohydrate; (ii) modulate complex formation with a prolactin and/or growth hormone receptor; (iii) regulate cellular growth; (iv) regulate cellular proliferation; (v) regulate angiogenesis; (vi) regulate cellular differentiation; and (vii) regulate cell survival, either in vitro or in vivo.

The isolated nucleic acid molecules of the invention can be used, for example, to express EDIRF, MTR-1, LSP-1, TAP-1, or PA-I protein (e.g., via a recombinant expression vector in a host cell in gene therapy applications), to detect EDIRF, MTR-1, LSP-1, TAP-1, or PA-I mRNA (e.g., in a biological sample) or a genetic alteration in an EDIRF, MTR-1, LSP-1, TAP-1, or PA-I gene, and to modulate EDIRF, MTR-1, LSP-1, TAP-1, or PA-I activity, as described further below. In addition, the EDIRF, MTR-1, LSP-1, TAP-1, or PA-I proteins can be used to screen drugs or compounds which modulate the EDIRF, MTR-1, LSP-1, TAP-1, or PA-I activity as well as to treat disorders characterized by insufficient or excessive production of EDIRF, MTR-1, LSP-1, TAP-1, or PA-I protein or production of EDIRF, MTR-1, LSP-1, TAP-1, or PA-I protein forms which have decreased or aberrant activity compared to EDIRF, MTR-1, LSP-1, TAP-1, or PA-I wild type protein (e.g., inflammatory diseases such as arthritis, differentiative or developmental disorders, or immune response disorders; insulin resistance seen with noninsulin dependent diabetes mellitus and obesity; hematopoietic disorders disorders such as thrombocytopenia or anemia; or proliferative disorders such as cancer or angiogenesis related disorders). Moreover, soluble forms of the EDIRF, MTR-1, LSP-1, TAP-1, or PA-I protein can be used to bind membrane-bound EDIRF, MTR-1, LSP-1, TAP-1, or PA-I or other membrane-bound cytokine receptors and influence bioavailability of EDIRF, MTR-1, LSP-1, TAP-1, or PA-I ligands or the cognate ligands of other membrane-bound receptors. In addition, the anti-EDIRF, MTR-1, LSP-1, TAP-1, or PA-I antibodies of the invention can be used to detect and isolate EDIRF, MTR-1, LSP-1, TAP-1, or PA-I proteins and modulate EDIRF, MTR-1, LSP-1, TAP-1, or PA-I activity.

It will therefore be appreciated that the EDIRF, MTR-1, LSP-1, TAP-1, or PA-I polypeptides, nucleic acids, and modulators thereof can be used to modulate the function, morphology, proliferation and/or differentiation of cells in the tissues in which it is expressed, as described herein. Such molecules can thus be used to treat disorders associated with abnormal or aberrant metabolism or function of cells in the tissues in which it is expressed.

For example, when EDIRF, MTR-1, LSP-1, TAP-1, or PA-I polypeptides and nucleic acids are expressed in the pancreas, the polypeptides, nucleic acids, and modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

In another example, when EDIRF, MTR-1, LSP-1, TAP-1, or PA-I polypeptides and nucleic acids are expressed in the heart, the polypeptides, nucleic acids, and modulators thereof can be used to treat heart disorders, e.g., ischemic heart disease, atherosclerosis, hypertension, angina pectoris, Hypertrophic Cardiomyopathy, and congenital heart disease.

In still another example, the EDIRF, MTR-1, LSP-1, TAP-1, or PA-I polypeptides, nucleic acids, and modulators thereof may be used to modulate the pituitary gland, which secretes such hormones as thyroid stimulating hormone (TSH), follicle stimulating hormone (FSH), adrenocotropic hormone (ACTH), and others. It controls the activity of many other endocrine glands (thyroid, ovaries, adrenal, etc.). Pituitary related disorders include, among others, acromegaly, Cushing's syndrome, craniopharyngiomas, Empty Sella syndrome, hypogonadism, hypopituitarism, and hypophysitis, in addition to disorders of the endocrine glands the pituitary controls.

In another example, the EDIRF, MTR-1, LSP-1, TAP-1, or PA-I polypeptides, nucleic acids, and modulators thereof can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

In another example, the EDIRF, MTR-1, LSP-1, TAP-1, or PA-I polypeptides, nucleic acids, and modulators thereof can be used to treat disorders of the adrenal medulla, such as neoplasms (e.g., pheochromocytomas, neuroblastomas, and ganglioneuromas).

In another example, the EDIRF, MTR-1, LSP-1, TAP-1, or PA-I polypeptides, nucleic acids, and modulators thereof can be used to treat disorders of the thyroid gland, such as hyperthyroidism (e.g., diffuse toxic hyperplasia, toxic multinodular goiter, toxic adenoma, and acute or subacute thyroiditis), hypothyroidism (e.g., cretinism and myxedema), thyroiditis (e.g., Hashimoto's thyroiditis, subacute granulomatous thyroiditis, subacute lymphocytic thyroiditis, Riedel's thryroiditis), Graves' disease, goiter (e.g., simple diffuse goiter and multinodular goiter), or tumors (e.g., adenoma, papillary carcinoma, follicular carcinoma, medullary carcinoma, undifferentiated malignant carcinoma, Hodgkin's disease, and non-Hodgkin's lymphoma).

In another example, the EDIRF, MTR-1, LSP-1, TAP-1, or PA-I polypeptides, nucleic acids, and modulators thereof can be used to treat gastric disorders, such as congenital anomalies (e.g., diaphragmatic hernias, pyloric stenosis, gastric diverticula, and gastric dilatation), gastritis (e.g., acute mucosal inflammation, chronic fundal gastritis, chronic antral gastritis, hypertrophic gastritis, granulomatous gastritis, eosinophilic gastritis), ulcerations (e.g., peptic ulcers, gastric ulcers, and duodenal ulcers), or tumors (e.g., benign polyps, malignant carcinoma, argentaffinomas, carcinoids, gastrointestinal lymphomas, carcomas, and metastatic carcinoma).

In another example, the EDIRF, MTR-1, LSP-1, TAP-1, or PA-I polypeptides, nucleic acids, and modulators thereof can be used to treat placental disorders, such as toxemia of pregnancy (e.g., preeclampsia and eclampsia), placentitis, or spontaneous abortion.

In another example, the EDIRF, MTR-1, LSP-1, TAP-1, or PA-I polypeptides, nucleic acids, and modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis, Goodpasture's syndrome, idiopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another example, the EDIRF, MTR-1, LSP-1, TAP-1, or PA-I polypeptides, nucleic acids, and modulators thereof can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, the EDIRF, MTR-1, LSP-1, TAP-1, or PA-I polypeptides, nucleic acids, and modulators thereof can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

In another example, the EDIRF, MTR-1, LSP-1, TAP-1, or PA-I polypeptides, nucleic acids, and modulators thereof can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis), hepatitis (e.g., chronic active hepatitis, acute viral hepatitis, and toxic and drug-induced hepatitis), cirrhosis (e.g., alcoholic cirrhosis, biliary cirrhosis, and hemochromatosis), or malignant tumors (e.g., primary carcinoma, hepatoma, hepatoblastoma, liver cysts, and angiosarcoma).

In another example, the EDIRF, MTR-1, LSP-1, TAP-1, or PA-I polypeptides, nucleic acids, and modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, the EDIRF, MTR-1, LSP-1, TAP-1, or PA-I polypeptides, nucleic acids, and modulators thereof can be used to treat testicular disorders, such as unilateral testicular enlargement (e.g., nontuberculous, granulomatous orchitis); inflammatory diseases resulting in testicular dysfunction (e.g., gonorrhea and mumps); cryptorchidism; sperm cell disorders (e.g., immotile cilia syndrome and germinal cell aplasia); acquired testicular defects (e.g., viral orchitis); and tumors (e.g., germ cell tumors, interstitial cell tumors, androblastoma, testicular lymphoma and adenomatoid tumors).

In another example, the EDIRF, MTR-1, LSP-1, TAP-1, or PA-I polypeptides, nucleic acids, and modulators thereof can be used to treat uterine disorders, e.g., hyperplasia of the endometrium, uterine cancers (e.g., uterine leiomyomoma, uterine cellular leiomyoma, leiomyosarcoma of the uterus, malignant mixed mullerian Tumor of uterus, uterine Sarcoma), and dysfunctional uterine bleeding (DUB).

In another example, EDIRF, MTR-1, LSP-1, TAP-1, or PA-I polypeptides, nucleic acids, and modulators thereof can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, EDIRF, MTR-1, LSP-1, TAP-1, or PA-I nucleic acids, proteins, and modulators thereof can be used to modulate or treat disorders that include hepatitis B as well as brain and CNS related disorders. Such brain and CNS related disorders include but are not limited to bacterial and viral meningitis, Alzheimers Disease, cerebral toxoplasmosis, Parkinson's disease, multiple sclerosis, brain cancers (e.g., metastatic carcinoma of the brain, glioblastoma, lymphoma, astrocytoma, acoustic neuroma), hydrocephalus, and encephalitis.

In another example, EDIRF, MTR-1, LSP-1, TAP-1, or PA-I nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. EDIRF, MTR-1, LSP-1, TAP-1, or PA-I nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus EDIRF, MTR-1, LSP-1, TAP-1, or PA-I nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

In another example, EDIRF, MTR-1, LSP-1, TAP-1, or PA-I nucleic acids, proteins, and modulators thereof can be used to modulate the proliferation, differentiation, and/or function of cells that form bone matrix, e.g., osteoblasts and osteoclasts, and can be used to modulate the formation of bone matrix. Thus EDIRF, MTR-1, LSP-1, TAP-1, or PA-I nucleic acids, proteins, and modulators thereof can be used to treat cartilage and bone associated diseases and disorders, and can play a role in bone growth, formation, and remodeling. Examples of cartilage and bone associated diseases and disorders include e.g., bone cancer, achondroplasia, myeloma, fibrous dysplasia, scoliosis, osteoarthritis, osteosarcoma, and osteoporosis.

In another example, EDIRF, MTR-1, LSP-1, TAP-1, or PA-I nucleic acids, proteins, and modulators thereof can be used to modulate the proliferation, differentiation, and/or function of cells that appear in the bone marrow, e.g., stem cells (e.g., hematopoietic stem cells), and blood cells, e.g., erythrocytes, platelets, and leukocytes. Thus EDIRF, MTR-1, LSP-1, TAP-1, or PA-I nucleic acids, proteins, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

In another example, EDIRF, MTR-1, LSP-1, TAP-1, or PA-I nucleic acids, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

In another example, EDIRF, MTR-1, LSP-1, TAP-1, or PA-I polypeptides, nucleic acids, and modulators thereof can be used to treat esophageal and other digestive system related disorders, e.g., dysphagia (e.g., oropharyngeal dysphagia, esophageal dysphagia), pyrosis, achalasia, diffuse esophageal spasm, nutcracker esophagus, and gastroesophageal refulx disease.

In another example, EDIRF, MTR-1, LSP-1, TAP-1, or PA-I polypeptides, nucleic acids, and modulators thereof can be used to treat eye disorders, e.g., Retinitis Pigmentosa, Cataract, Color Blindness, Conjunctivitis, Dry Eyes, Glaucoma, Keratoconus, Macular Degeneration, Microphthalmia and Anophthalmia, Myopia, Nystagmus, Retinitis Pigmentosa, and Trachoma.

In another example, EDIRF, MTR-1, LSP-1, TAP-1, or PA-I polypeptides, nucleic acids, and modulators thereof can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

In another example, EDIRF, MTR-1, LSP-1, TAP-1, or PA-I polypeptides, nucleic acids, and modulators thereof can be used to treat ovarian disorders, such as ovarian endometriosis, non-neoplastic cysts (e.g., follicular and luteal cysts and polycystic ovaries) and tumors (e.g., tumors of surface epithelium, germ cell tumors, ovarian fibroma, sex cord-stromal tumors, and ovarian cancers (e.g., metastatic carcinomas, and ovarian teratoma).

In another example, EDIRF, MTR-1, LSP-1, TAP-1, or PA-I polypeptides, nucleic acids, and modulators thereof can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, EDIRF, MTR-1, LSP-1, TAP-1, or PA-I polypeptides, nucleic acids, and modulators thereof can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., colitis (e.g., idiopathic ulcerative colitis, pseudomembranous colitis), and lymphopathia venereum), Crohn's disease, and tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas).

In another example, EDIRF, MTR-1, LSP-1, TAP-1, or PA-I polypeptides, nucleic acids, and modulators thereof can be used to treat leukocytic disorders, such as leukopenias (e.g., neutropenia, monocytopenia, lymphopenia, and granulocytopenia), leukocytosis (e.g., granulocytosis, lymphocytosis, eosinophilia, monocytosis, acute and chronic lymphadenitis), malignant lymphomas (e.g., Non-Hodgkin's lymphomas, Hodgkin's lymphomas, leukemias, agnogenic myeloid metaplasia, multiple myeloma, plasmacytoma, Waldenstrom's macroglobulinemia, heavy-chain disease, monoclonal gammopathy, histiocytoses, eosinophilic granuloma, and angioimmunoblastic lymphadenopathy).

A. Screening Assays:

The invention provides a method (also referred to herein as a “screening assay”) for identifying modulators, i.e., candidate or test compounds or agents (e.g., peptides, peptidomimetics, small molecules or other drugs) which bind to EDIRF, MTR-1, LSP-1, TAP-1, or PA-I proteins or have a stimulatory or inhibitory effect on, for example, EDIRF, MTR-1, LSP-1, TAP-1, or PA-I expression or EDIRF, MTR-1, LSP-1, TAP-1, or PA-I activity.

In one embodiment, the invention provides assays for screening candidate or test compounds which bind to or modulate the activity of an EDIRF, MTR-1, LSP-1, TAP-1, or PA-I protein or polypeptide or biologically active portion thereof. In another embodiment, the invention provides assays for screening candidate or test compounds which bind to or modulate the activity of an EDIRF, MTR-1, LSP-1, TAP-1, or PA-I receptor. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an EDIRF, MTR-1, LSP-1, TAP-1, or PA-I receptor on the cell surface is contacted with a test compound and the ability of the test compound to bind to an EDIRF, MTR-1, LSP-1, TAP-1, or PA-I receptor determined. The cell, for example, can be of mammalian origin or a yeast cell. Determining the ability of the test compound to bind to an EDIRF, MTR-1, LSP-1, TAP-1, or PA-I receptor can be accomplished, for example, by coupling the test compound with a radioisotope or enzymatic label such that binding of the test compound to the EDIRF, MTR-1, LSP-1, TAP-1, or PA-I receptor can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with an EDIRF, MTR-1, LSP-1, TAP-1, or PA-I receptor without the labeling of any of the interactants. For example, a microphysiometer can be used to detect the interaction of a test compound with an EDIRF, MTR-1, LSP-1, TAP-1, or PA-I receptor without the labeling of either the test compound or the receptor. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses an EDIRF, MTR-1, LSP-1, TAP-1, or PA-I receptor on the cell surface with an EDIRF, MTR-1, LSP-1, TAP-1, or PA-I protein or biologically-active portion thereof, to form an assay mixture, contacting the assay mixture with a test compound, and determining the ability of the test compound to interact with an EDIRF, MTR-1, LSP-1, TAP-1, or PA-I receptor, wherein determining the ability of the test compound to interact with an EDIRF, MTR-1, LSP-1, TAP-1, or PA-I receptor comprises determining the ability of the test compound to preferentially bind to the EDIRF, MTR-1, LSP-1, TAP-1, or PA-I receptor as compared to the ability of EDIRF, MTR-1, LSP-1, TAP-1, or PA-I, or a biologically active portion thereof, to bind to the receptor.

In another embodiment, an assay is a cell-based assay comprising contacting a cell expressing an EDIRF, MTR-1, LSP-1, TAP-1, or PA-I target molecule with a test compound and determining the ability of the test compound to modulate (e.g. stimulate or inhibit) the activity of the EDIRF, MTR-1, LSP-1, TAP-1, or PA-I target molecule. Determining the ability of the test compound to modulate the activity of EDIRF, MTR-1, LSP-1, TAP-1, or PA-I target molecule can be accomplished, for example, by determining the ability of the EDIRF, MTR-1, LSP-1, TAP-1, or PA-I protein to bind to or interact with the EDIRF, MTR-1, LSP-1, TAP-1, or PA-I target molecule. As used herein, a “target molecule” is a molecule with which an EDIRF, MTR-1, LSP-1, TAP-1, or PA-I protein binds or interacts in nature, for example, a molecule on the surface of a cell which expresses an EDIRF, MTR-1, LSP-1, TAP-1, or PA-I protein, a molecule on the surface of a second cell, or a molecule in the extracellular milieu. An EDIRF, MTR-1, LSP-1, TAP-1, or PA-I target molecule can be a non-EDIRF, non-MTR-1, non-LSP-1, non-TAP-1, or non-PA-I molecule or an EDIRF, MTR-1, LSP-1, TAP-1, or PA-I protein or polypeptide of the present invention. The target, for example, can be a membrane-bound or membrane-associated protein which facilitates signal transduction from EDIRF, MTR-1, LSP-1, TAP-1, or PA-I to an intercellular protein which has catalytic activity or to an intercellular protein which facilitates the association of downstream signaling molecules with EDIRF, MTR-1, LSP-1, TAP-1, or PA-I.

Determining the ability of the EDIRF, MTR-1, LSP-1, TAP-1, or PA-I protein to bind to or interact with an EDIRF, MTR-1, LSP-1, TAP-1, or PA-I target molecule can be accomplished by one of the methods described above for determining direct binding. In a preferred embodiment, determining the ability of the EDIRF, MTR-1, LSP-1, TAP-1, or PA-I protein to bind to or interact with an EDIRF, MTR-1, LSP-1, TAP-1, or PA-I target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an EDIRF, MTR-1, LSP-1, TAP-1, or PA-I-responsive regulatory element operatively linked to a nucleic acid encoding a detectable marker, e.g., luciferase), or detecting a cellular response, for example, an immune cell response or cell homing.

In yet another embodiment, an assay of the present invention is a cell-free assay in which an EDIRF, MTR-1, LSP-1, TAP-1, or PA-I protein or biologically active portion thereof is contacted with a test compound and the ability of the test compound to bind to the EDIRF, MTR-1, LSP-1, TAP-1, or PA-I protein or biologically active portion thereof is determined. Binding of the test compound to the EDIRF, MTR-1, LSP-1, TAP-1, or PA-I protein can be determined either directly or indirectly as described above. In a preferred embodiment, the assay includes contacting the EDIRF, MTR-1, LSP-1, TAP-1, or PA-I protein or biologically active portion thereof with a known compound which binds EDIRF, MTR-1, LSP-1, TAP-1, or PA-I to form an assay mixture, contacting the assay mixture with a test compound, and determining the ability of the test compound to interact with an EDIRF, MTR-1, LSP-1, TAP-1, or PA-I protein, wherein determining the ability of the test compound to interact with an EDIRF, MTR-1, LSP-1, TAP-1, or PA-I protein comprises determining the ability of the test compound to preferentially bind to EDIRF, MTR-1, LSP-1, TAP-1, or PA-I or biologically active portion thereof as compared to the known compound.

In another embodiment, the assay is a cell-free assay in which EDIRF, MTR-1, LSP-1, TAP-1, or PA-I protein or biologically active portion thereof is contacted with a test compound and the ability of the test compound to modulate (e.g., stimulate or inhibit) the activity of the EDIRF, MTR-1, LSP-1, TAP-1, or PA-I protein or biologically active portion thereof is determined. Determining the ability of the test compound to modulate the activity of EDIRF, MTR-1, LSP-1, TAP-1, or PA-I can be accomplished, for example, by determining the ability of the EDIRF, MTR-1, LSP-1, TAP-1, or PA-I protein to bind to an EDIRF, MTR-1, LSP-1, TAP-1, or PA-I target molecule by one of the methods described above for determining direct binding. Determining the ability of the EDIRF, MTR-1, LSP-1, TAP-1, or PA-I protein to bind to an EDIRF, MTR-1, LSP-1, TAP-1, or PA-I target molecule can also be accomplished using a technology such as real-time Biomolecular Interaction Analysis (BIA). Sjolander, S. and Urbaniczky, C. (1991) Anal Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of EDIRF, MTR-1, LSP-1, TAP-1, or PA-I can be accomplished by determining the ability of the EDIRF, MTR-1, LSP-1, TAP-1, or PA-I protein to further modulate an EDIRF, MTR-1, LSP-1, TAP-1, or PA-I target molecule. For example, the catalytic/enzymatic activity of the target molecule on an appropriate substrate can be determined as previously described.

In yet another embodiment, the cell-free assay involves contacting the EDIRF, MTR-1, LSP-1, TAP-1, or PA-I protein or biologically active portion thereof with a known compound which binds EDIRF, MTR-1, LSP-1, TAP-1, or PA-I to form an assay mixture, contacting the assay mixture with a test compound, and determining the ability of the test compound to interact with an EDIRF, MTR-1, LSP-1, TAP-1, or PA-I protein, wherein determining the ability of the test compound to interact with an EDIRF, MTR-1, LSP-1, TAP-1, or PA-I protein comprises determining the ability of the EDIRF, MTR-1, LSP-1, TAP-1, or PA-I protein to preferentially bind to or modulate the activity of an EDIRF, MTR-1, LSP-1, TAP-1, or PA-I target molecule.

The cell-free assays of the present invention are amenable to use of both soluble and/or membrane-bound forms of isolated proteins (e.g. EDIRF, MTR-1, LSP-1, TAP-1, or PA-I proteins or biologically active portions thereof or EDIRF, MTR-1, LSP-1, TAP-1, or PA-I target molecules). In the case of cell-free assays in which a membrane-bound form an isolated protein is used (e.g., an EDIRF, MTR-1, LSP-1, TAP-1, or PA-I target molecule or receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-1 14, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either EDIRF, MTR-1, LSP-1, TAP-1, or PA-I or its target molecule to facilitate separation of complexed from uncomplexed forms of one or both of the proteins, as well as to accommodate automation of the assay. Binding of a test compound to EDIRF, MTR-1, LSP-1, TAP-1, or PA-I, or interaction of EDIRF, MTR-1, LSP-1, TAP-1, or PA-I with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/EDIRF, MTR-1, LSP-1, TAP-1, or PA-I fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or EDIRF, MTR-1, LSP-1, TAP-1, or PA-I protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of EDIRF, MTR-1, LSP-1, TAP-1, or PA-I binding or activity determined using standard techniques.

Other techniques for immobilizing proteins on matrices can also be used in the screening assays of the invention. For example, either EDIRF, MTR-1, LSP-1, TAP-1, or PA-I or its target molecule can be immobilized utilizing conjugation of biotin and streptavidin. Biotinylated EDIRF, MTR-1, LSP-1, TAP-1, or PA-I or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with EDIRF, MTR-1, LSP-1, TAP-1, or PA-I or target molecules but which do not interfere with binding of the EDIRF, MTR-1, LSP-l, TAP-1, or PA-I protein to its target molecule can be derivatized to the wells of the plate, and unbound target or EDIRF, MTR-1, LSP-1, TAP-1, or PA-I trapped in the wells by antibody conjugation. Methods for detecting such complexes, in addition to those described above for the GST-immobilized complexes, include immunodetection of complexes using antibodies reactive with the EDIRF, MTR-1, LSP-1, TAP-1, or PA-I or target molecule, as well as enzyme-linked assays which rely on detecting an enzymatic activity associated with the EDIRF, MTR-1, LSP-1, TAP-1, or PA-I or target molecule.

In another embodiment, modulators of EDIRF, MTR-1, LSP-l, TAP-1, or PA-I expression are identified in a method wherein a cell is contacted with a candidate compound and the expression of EDIRF, MTR-1, LSP-1, TAP-1, or PA-I mRNA or protein in the cell is determined. The level of expression of EDIRF, MTR-1, LSP-1, TAP-1, or PA-I mRNA or protein in the presence of the candidate compound is compared to the level of expression of EDIRF, MTR-1, LSP-1, TAP-1, or PA-I mRNA or protein in the absence of the candidate compound. The candidate compound can then be identified as a modulator of EDIRF, MTR-1, LSP-1, TAP-1, or PA-I expression based on this comparison. For example, when expression of EDIRF, MTR-1, LSP-1, TAP-1, or PA-I mRNA or protein is greater (statistically significantly greater) in the presence of the candidate compound than in its absence, the candidate compound is identified as a stimulator of EDIRF, MTR-1, LSP-1, TAP-1, or PA-I mRNA or protein expression. Alternatively, when expression of EDIRF, MTR-1, LSP-1, TAP-1, or PA-I mRNA or protein is less (statistically significantly less) in the presence of the candidate compound than in its absence, the candidate compound is identified as an inhibitor of EDIRF, MTR-1, LSP-1, TAP-1, or PA-I mRNA or protein expression. The level of EDIRF, MTR-1, LSP-1, TAP-1, or PA-I mRNA or protein expression in the cells can be determined by methods described herein for detecting EDIRF, MTR-1, LSP-1, TAP-1, or PA-I mRNA or protein.

In yet another aspect of the invention, the EDIRF, MTR-1, LSP-1, TAP-1, or PA-I proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with EDIRF, MTR-1, LSP-1, TAP-1, or PA-I (“EDIRF, MTR-1, LSP-1, TAP-1, or PA-I-binding proteins” or “EDIRF, MTR-1, LSP-1, TAP-1, or PA-I -bp”) and modulate EDIRF, MTR-1, LSP-1, TAP-1, or PA-I activity. Such EDIRF, MTR-1, LSP-1, TAP-1, or PA-I -binding proteins are also likely to be involved in the propagation of signals by the EDIRF, MTR-1, LSP-1, TAP-1, or PA-I proteins as, for example, downstream elements of an EDIRF, MTR-1, LSP-1, TAP-1, or PA-I -mediated signaling pathway. Alternatively, such EDIRF, MTR-1, LSP-1, TAP-1, or PA-I -binding proteins are likely to be cell-surface molecules associated with non-EDIRF, non-MTR-1, non-LSP-l, non-TAP-1, or non-PA-I expressing cells, wherein such EDIRF, MTR-1, LSP-1, TAP-1, or PA-I -binding proteins are involved in secondary cytokine production.

The two-hybrid system is based on the modular nature of most transcription factors, which consist of separable DNA-binding and activation domains. Briefly, the assay utilizes two different DNA constructs. In one construct, the gene that codes for EDIRF, MTR-1, LSP-1, TAP-1, or PA-I is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an EDIRF, MTR-1, LSP-1, TAP-1, or PA-I -dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with EDIRF, MTR-1, LSP-1, TAP-1, or PA-I.

This invention further pertains to novel agents identified by the above-described screening assays. Accordingly, it is within the scope of this invention to further use an agent identified as described herein in an appropriate animal model. For example, an agent identified as described herein (e.g., an EDIRF, MTR-1, LSP-1, TAP-1, or PA-I modulating agent, an antisense EDIRF, MTR-1, LSP-1, TAP-1, or PA-I nucleic acid molecule, an EDIRF, MTR-1, LSP-1, TAP-1, or PA-I -specific antibody, or an EDIRF, MTR-1, LSP-1, TAP-1, or PA-I binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the EDIRF, MTR-1, LSP-1, TAP-1, or PA-I, sequences, described herein, can be used to map the location of the EDIRF, MTR-1, LSP-1, TAP-1, or PA-I genes, respectively, on a chromosome. The mapping of the EDIRF, MTR-1, LSP-1, TAP-1, or PA-I sequences to chromosomes is an important first step in correlating these sequences with genes associated with disease.

For example, the LSP-1 gene maps to chromosome 7q21-q22, at 111-112 cM, very close to the Erythropoietin (EPO) gene.

Briefly, EDIRF, MTR-1, LSP-1, TAP-1, or PA-I genes can be mapped to chromosomes by preparing PCR primers (preferably 15-25 bp in length) from the EDIRF, MTR-1, LSP-1, TAP-1, or PA-I sequences. Computer analysis of the EDIRF, MTR-1, LSP-1, TAP-1, or PA-I, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the EDIRF, MTR-1, LSP-1, TAP-1, or PA-I sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the EDIRF, MTR-1, LSP-1, TAP-1, or PA-I sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, for example, Egeland, J. et al. (1987) Nature 325:783-787.

Moreover, differences in the DNA sequences between individuals affected and unaffected with a disease associated with the EDIRF, MTR-1, LSP-1, TAP-1, or PA-I gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The EDIRF, MTR-1, LSP-1, TAP-1, and PA-I sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the EDIRF, MTR-1, LSP-1, TAP-1, or PA-I sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The EDIRF, MTR-1, LSP-1, TAP-1, and PA-I sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:NO:1, 3, 5, 34, 43, 49, or 53 can comfortably provide positive individual identification with a panel of perhaps 10 to 1,000 primers which each yield a noncoding amplified sequence of 100 bases. If predicted coding sequences, such as those in SEQ ID NO:7, 8, 9, 36, 45, 51, or 55 are used, a more appropriate number of primers for positive individual identification would be 500-2,000.

If a panel of reagents from EDIRF, MTR-1, LSP-1, TAP-1, or PA-I sequences described herein is used to generate a unique identification database for an individual, those same reagents can later be used to identify tissue from that individual. Using the unique identification database, positive identification of the individual, living or dead, can be made from extremely small tissue samples.

3. Use of Partial Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NOs:1, SEQ ID NO:3 or SEQ ID NO:5 are particularly appropriate for this use as greater numbers of polymorphisms occur in the noncoding regions, making it easier to differentiate individuals using this technique. Examples of polynucleotide reagents include the EDIRF, MTR-1, LSP-1, TAP-1, or PA-I sequences or portions thereof, e.g., fragments derived from the noncoding regions of SEQ ID NO:1, 3, 5, 34, 43, 49, or 53, having a length of at least 20 bases, preferably at least 30 bases.

The EDIRF, MTR-1, LSP-1, TAP-1, or PA-I sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such EDIRF, MTR-1, LSP-1, TAP-1, or PA-I probes can be used to identify tissue by species and/or by organ type.

In a similar fashion, these reagents, e.g., EDIRF, MTR-1, LSP-1, TAP-1, or PA-I primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining EDIRF, MTR-1, LSP-1, TAP-1, or PA-I protein and/or nucleic acid expression as well as EDIRF, MTR-1, LSP-1, TAP-1, or PA-I activity, in the context of a biological sample (e.g., blood, serum, cells, tissue) to thereby determine whether an individual is afflicted with a disease or disorder, or is at risk of developing a disorder, associated with aberrant EDIRF, MTR-1, LSP-1, TAP-1, or PA-I expression or activity. The invention also provides for prognostic (or predictive) assays for determining whether an individual is at risk of developing a disorder associated with EDIRF, MTR-1, LSP-1, TAP-1, or PA-I protein, nucleic acid expression or activity. For example, mutations in an EDIRF, MTR-1, LSP-1, TAP-1, or PA-I gene can be assayed in a biological sample. Such assays can be used for prognostic or predictive purpose to thereby prophylactically treat an individual prior to the onset of a disorder characterized by or associated with EDIRF, MTR-1, LSP-1, TAP-1, or PA-I protein, nucleic acid expression or activity.

Another aspect of the invention pertains to monitoring the influence of agents (e.g., drugs, compounds) on the expression or activity of EDIRF, MTR-1, LSP-1, TAP-1, or PA-I in clinical trials.

These and other agents are described in further detail in the following sections.

1. Diagnostic Assays

An exemplary method for detecting the presence or absence of EDIRF, MTR-1, LSP-1, TAP-1, or PA-I in a biological sample involves obtaining a biological sample from a test subject and contacting the biological sample with a compound or an agent capable of detecting EDIRF, MTR-1, LSP-1, TAP-1, or PA-I protein or nucleic acid (e.g., mRNA, genomic DNA) that encodes EDIRF, MTR-1, LSP-1, TAP-1, or PA-I protein such that the presence of EDIRF, MTR-1, LSP-1, TAP-1, or PA-I is detected in the biological sample. A preferred agent for detecting EDIRF, MTR-1, LSP-1, TAP-1, or PA-I mRNA or genomic DNA is a labeled nucleic acid probe capable of hybridizing to EDIRF, MTR-1, LSP-1, TAP-1, or PA-I mRNA or genomic DNA. The nucleic acid probe can be, for example, a full-length EDIRF, MTR-1, LSP-1, TAP-1, or PA-I nucleic acid, such as the nucleic acid of SEQ ID NO:1, 3, 5, 7, 8, 9, 34, 36, 43, 49, 51, 53, or 55, the DNA insert of the plasmid deposited with ATCC as Accession Number 98613, 98614, 98550, or 98554, or a portion thereof, such as an oligonucleotide of at least 15, 30, 50, 100, 250 or 500 nucleotides in length and sufficient to specifically hybridize under stringent conditions to EDIRF, MTR-1, LSP-1, TAP-1, or PA-I mRNA or genomic DNA. Other suitable probes for use in the diagnostic assays of the invention are described herein.

A preferred agent for detecting EDIRF, MTR-1, LSP-1, TAP-1, or PA-I protein is an antibody capable of binding to EDIRF, MTR-1, LSP-1, TAP-1, or PA-I protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect EDIRF, MTR-1, LSP-1, TAP-1, or PA-I mRNA, protein, or genomic DNA in a biological sample in vitro as well as in vivo. For example, in vitro techniques for detection of EDIRF, MTR-1, LSP-1, TAP-1, or PA-I mRNA include Northern hybridizations and in situ hybridizations. In vitro techniques for detection of EDIRF, MTR-1, LSP-1, TAP-1, or PA-I protein include enzyme linked immunosorbent assays (ELISAs), Western blots, immunoprecipitations and immunofluorescence. In vitro techniques for detection of EDIRF, MTR-1, LSP-1, TAP-1, or PA-I genomic DNA include Southern hybridizations. Furthermore, in vivo techniques for detection of EDIRF, MTR-1, LSP-1, TAP-1, or PA-I protein include introducing into a subject a labeled anti-EDIRF, MTR-1, LSP-1, TAP-1, or PA-I antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting EDIRF, MTR-1, LSP-1, TAP-1, or PA-I protein, mRNA, or genomic DNA, such that the presence of EDIRF, MTR-1, LSP-1, TAP-1, or PA-I protein, mRNA or genomic DNA is detected in the biological sample, and comparing the presence of EDIRF, MTR-1, LSP-1, TAP-1, or PA-I protein, mRNA or genomic DNA in the control sample with the presence of EDIRF, MTR-1, LSP-1, TAP-1, or PA-I protein, mRNA or genomic DNA in the test sample.

The invention also encompasses kits for detecting the presence of EDIRF, MTR-1, LSP-1, TAP-1, or PA-I in a biological sample. For example, the kit can comprise a labeled compound or agent capable of detecting EDIRF, MTR-1, LSP-1, TAP-1, or PA-I protein or mRNA in a biological sample; means for determining the amount of EDIRF, MTR-1, LSP-1, TAP-1, or PA-I in the sample; and means for comparing the amount of EDIRF, MTR-1, LSP-1, TAP-1, or PA-I in the sample with a standard. The compound or agent can be packaged in a suitable container. The kit can further comprise instructions for using the kit to detect EDIRF, MTR-1, LSP-1, TAP-1, or PA-I protein or nucleic acid.

2. Prognostic Assays

The diagnostic methods described herein can furthermore be utilized to identify subjects having or at risk of developing a disease or disorder associated with aberrant EDIRF, MTR-1, LSP-1, TAP-1, or PA-I expression or activity. For example, the assays described herein, such as the preceding diagnostic assays or the following assays, can be utilized to identify a subject having or at risk of developing a disorder associated with EDIRF, MTR-1, LSP-1, TAP-1, or PA-I protein, nucleic acid expression or activity such as inflammatory diseases such as arthritis, differentiative or developmental disorders, or immune response disorders; insulin resistance seen with noninsulin dependent diabetes mellitus and obesity; hematopoietic disorders disorders such as thrombocytopenia or anemia; or proliferative disorders such as cancer or angiogenesis related disorders. Alternatively, the prognostic assays can be utilized to identify a subject having or at risk for developing inflammatory diseases such as arthritis, differentiative or developmental disorders, or immune response disorders; insulin resistance seen with noninsulin dependent diabetes mellitus and obesity; hematopoietic disorders disorders such as thrombocytopenia or anemia; or proliferative disorders such as cancer or angiogenesis related disorders. Thus, the present invention provides a method for identifying a disease or disorder associated with aberrant EDIRF, MTR-1, LSP-1, TAP-1, or PA-I expression or activity in which a test sample is obtained from a subject and EDIRF, MTR-1, LSP-1, TAP-1, or PA-I protein or nucleic acid (e.g., mRNA, genomic DNA) is detected, wherein the presence of EDIRF, MTR-1, LSP-1, TAP-1, or PA-I protein or nucleic acid is diagnostic for a subject having or at risk of developing a disease or disorder associated with aberrant EDIRF, MTR-1, LSP-1, TAP-1, or PA-I expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant EDIRF, MTR-1, LSP-1, TAP-1, or PA-I expression or activity. For example, such methods can be used to determine whether a subject can be effectively treated with an agent for a disorder, such as inflammatory diseases such as arthritis, differentiative or developmental disorders, or immune response disorders; insulin resistance seen with noninsulin dependent diabetes mellitus and obesity; hematopoietic disorders disorders such as thrombocytopenia or anemia; or proliferative disorders such as cancer or angiogenesis related disorders. Alternatively, such methods can be used to determine whether a subject can be effectively treated with an agent for inflammatory diseases such as arthritis, differentiative or developmental disorders, or immune response disorders; insulin resistance seen with noninsulin dependent diabetes mellitus and obesity; hematopoietic disorders disorders such as thrombocytopenia or anemia; or proliferative disorders such as cancer or angiogenesis related disorders. Thus, the present invention provides methods for determining whether a subject can be effectively treated with an agent for a disorder associated with aberrant EDIRF, MTR-1, LSP-1, TAP-1, or PA-I expression or activity in which a test sample is obtained and EDIRF, MTR-1, LSP-1, TAP-1, or PA-I protein or nucleic acid is detected (e.g., wherein the presence of EDIRF, MTR-1, LSP-1, TAP-1, or PA-I protein or nucleic acid is diagnostic for a subject that can be administered the agent to treat a disorder associated with aberrant EDIRF, MTR-1, LSP-1, TAP-1, or PA-I expression or activity.)

The methods of the invention can also be used to detect genetic alterations in an EDIRF, MTR-1, LSP-1, TAP-1, or PA-I gene, thereby determining if a subject with the altered gene is at risk for a disorder characterized by aberrant development, aberrant immune responsiveness, an aberrant inflammatory response or an aberrant hematopoietic response, an aberrant insulin response; an aberrant hematopoietic response; or an aberrant proliferative response. In preferred embodiments, the methods include detecting, in a sample of cells from the subject, the presence or absence of a genetic alteration characterized by at least one of an alteration affecting the integrity of a gene encoding an EDIRF, MTR-1, LSP-1, TAP-1, or PA-I protein, or the mis-expression of the EDIRF, MTR-1, LSP-1, TAP-1, or PA-I gene. For example, such genetic alterations can be detected by ascertaining the existence of at least one of 1) a deletion of one or more nucleotides from an EDIRF, MTR-1, LSP-1, TAP-1, or PA-I gene; 2) an addition of one or more nucleotides to an EDIRF, MTR-1, LSP-1, TAP-1, or PA-I gene; 3) a substitution of one or more nucleotides of an EDIRF, MTR-1, LSP-1, TAP-1, or PA-I gene, 4) a chromosomal rearrangement of an EDIRF, MTR-1, LSP-1, TAP-1, or PA-I gene; 5) an alteration in the level of a messenger RNA transcript of an EDIRF, MTR-1, LSP-1, TAP-1, or PA-I gene, 6) aberrant modification of an EDIRF, MTR-1, LSP-1, TAP-1, or PA-I gene, such as of the methylation pattern of the genomic DNA, 7) the presence of a non-wild type splicing pattern of a messenger RNA transcript of an EDIRF, MTR-1, LSP-1, TAP-1, or PA-I gene, 8) a non-wild type level of an EDIRF, MTR-1, LSP-1, TAP-1, or PA-I protein, 9) allelic loss of an EDIRF, MTR-1, LSP-1, TAP-1, or PA-I gene, and 10) inappropriate post-translational modification of an EDIRF, MTR-1, LSP-1, TAP-1, or PA-I protein. As described herein, there are a large number of assay techniques known in the art which can be used for detecting alterations in an EDIRF, MTR-1, LSP-1, TAP-1, or PA-I gene. A preferred biological sample is serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the EDIRF, MTR-1, LSP-1, TAP-1, or PA-I gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to an EDIRF, MTR-1, LSP-1, TAP-1, or PA-I gene under conditions such that hybridization and amplification of the EDIRF, MTR-1, LSP-1, TAP-1, or PA-I gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an EDIRF, MTR-1, LSP-1, TAP-1, or PA-I gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in EDIRF, MTR-1, LSP-1, TAP-1, or PA-I can be identified by hybridizing a sample and control nucleic acids, e.g., DNA or RNA, to high density arrays containing hundreds or thousands of oligonucleotides probes (Cronin, M. T. et aL (1996) Human Mutation 7: 244-255; Kozal, M. J. et al. (1996) Nature Medicine 2: 753-759). For example, genetic mutations in EDIRF, MTR-1, LSP-1, TAP-1, or PA-I can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the EDIRF, MTR-1, LSP-1, TAP-1, or PA-I gene and detect mutations by comparing the sequence of the sample EDIRF, MTR-1, LSP-1, TAP-1, or PA-I with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the EDIRF, MTR-1, LSP-1, TAP-1, or PA-I gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type EDIRF, MTR-1, LSP-1, TAP-1, or PA-I sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci. USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in EDIRF, MTR-1, LSP-1, TAP-1, or PA-I cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an EDIRF, MTR-1, LSP-1, TAP-1, or PA-I sequence, e.g., a wild-type EDIRF, MTR-1, LSP-1, TAP-1, or PA-I sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in EDIRF, MTR-1, LSP-1, TAP-1, or PA-I genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet Anal Tech Appl 9:73-79). Single-stranded DNA fragments of sample and control EDIRF, MTR-1, LSP-1, TAP-1, or PA-I nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an EDIRF, MTR-1, LSP-1, TAP-1, or PA-I gene.

Furthermore, any cell type or tissue in which EDIRF, MTR-1, LSP-1, TAP-1, or PA-I is expressed may be utilized in the prognostic assays described herein.

3. Monitoring of Effects During Clinical Trials

Monitoring the influence of agents (e.g., drugs, compounds) on the expression or activity of EDIRF, MTR-1, LSP-1, TAP-1, or PA-I (e.g., activation of an EDIRF-dependent signal transduction pathway; modulation of secretion of a non-IL-17 cytokine; modulation of surface expression of a cellular adhesion molecule; modulation of a proinflammatory cytokine; modulation of a hematopoietic cytokine; the ability to transport metabolites across a cell membrane; the ability to modulate inflammation, immune responsiveness, or cellular homing; activation of an TAP-1-receptor; modulation of cellular proliferation or differentiation; modulation of maturation and/or differentiation of a hematopoietic stem cell; or modulation of thrombopoiesis) can be applied not only in basic drug screening, but also in clinical trials. For example, the effectiveness of an agent determined by a screening assay as described herein to increase EDIRF, MTR-1, LSP-1, TAP-1, or PA-I gene expression, protein levels, or upregulate EDIRF, MTR-1, LSP-1, TAP-1, or PA-I activity, can be monitored in clinical trails of subjects exhibiting decreased EDIRF, MTR-1, LSP-1, TAP-1, or PA-I gene expression, protein levels, or downregulated EDIRF, MTR-1, LSP-1, TAP-1, or PA-I activity. Alternatively, the effectiveness of an agent determined by a screening assay to decrease EDIRF, MTR-1, LSP-1, TAP-1, or PA-I gene expression, protein levels, or downregulate EDIRF, MTR-1, LSP-1, TAP-1, or PA-I activity, can be monitored in clinical trails of subjects exhibiting increased EDIRF, MTR-1, LSP-1, TAP-1, or PA-I gene expression, protein levels, or upregulated EDIRF, MTR-1, LSP-1, TAP-1, or PA-I activity. In such clinical trials, the expression or activity of EDIRF, MTR-1, LSP-1, TAP-1, or PA-I and, preferably, other genes that have been implicated in, for example, an immune response disorder can be used as a “read out” or markers of the immune responsiveness of a particular cell.

For example, and not by way of limitation, genes, including EDIRF, MTR-1, LSP-1, TAP-1, or PA-I, that are modulated in cells by treatment with an agent (e.g., compound, drug or small molecule) which modulates EDIRF, MTR-1, LSP-1, TAP-1, or PA-I activity (e.g., identified in a screening assay as described herein) can be identified. Thus, to study the effect of agents on immune response disorders, developmental disorder, or hematopoietic disorder, for example, in a clinical trial, cells can be isolated and RNA prepared and analyzed for the levels of expression of EDIRF, MTR-1, LSP-1, TAP-1, or PA-I and other genes implicated in the immune response disorders, developmental disorder, or hematopoietic disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of EDIRF, MTR-1, LSP-1, TAP-1, or PA-I or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of an EDIRF, MTR-1, LSP-1, TAP-1, or PA-I protein, mRNA, or genomic DNA in the preadministration sample; (iii) obtaining one or more post-administration samples from the subject; (iv) detecting the level of expression or activity of the EDIRF, MTR-1, LSP-1, TAP-1, or PA-I protein, mRNA, or genomic DNA in the post-administration samples; (v) comparing the level of expression or activity of the EDIRF, MTR-1, LSP-1, TAP-1, or PA-I protein, mRNA, or genomic DNA in the pre-administration sample with the EDIRF, MTR-1, LSP-1, TAP-1, or PA-I protein, mRNA, or genomic DNA in the post administration sample or samples; and (vi) altering the administration of the agent to the subject accordingly. For example, increased administration of the agent may be desirable to increase the expression or activity of EDIRF, MTR-1, LSP-1, TAP-1, or PA-I to higher levels than detected, i.e., to increase the effectiveness of the agent. Alternatively, decreased administration of the agent may be desirable to decrease expression or activity of EDIRF, MTR-1, LSP-1, TAP-1, or PA-I to lower levels than detected, i.e. to decrease the effectiveness of the agent. According to such an embodiment, EDIRF, MTR-1, LSP-1, TAP-1, or PA-I expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant EDIRF, MTR-1, LSP-1, TAP-1, or PA-I expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the EDIRF, MTR-1, LSP-1, TAP-1, or PA-I molecules of the present invention or EDIRF, MTR-1, LSP-1, TAP-1, or PA-I modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant EDIRF, MTR-1, LSP-1, TAP-1, or PA-I expression or activity, by administering to the subject an agent which modulates EDIRF, MTR-1, LSP-1, TAP-1, or PA-I expression or at least one EDIRF, MTR-1, LSP-1, TAP-1, or PA-I activity. Subjects at risk for a disease which is caused or contributed to by aberrant EDIRF, MTR-1, LSP-1, TAP-1, or PA-I expression or activity can be identified by, for example, any or a combination of diagnostic or prognostic assays as described herein. Administration of a prophylactic agent can occur prior to the manifestation of symptoms characteristic of the EDIRF, MTR-1, LSP-1, TAP-1, or PA-I aberrancy, such that a disease or disorder is prevented or, alternatively, delayed in its progression. Depending on the type of EDIRF, MTR-1, LSP-1, TAP-1, or PA-I aberrancy, for example, an EDIRF, MTR-1, LSP-1, TAP-1, or PA-I agonist or EDIRF, MTR-1, LSP-1, TAP-1, or PA-I antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating EDIRF, MTR-1, LSP-1, TAP-1, or PA-I expression or activity for therapeutic purposes. The modulatory method of the invention involves contacting a cell with an agent that modulates one or more of the activities of EDIRF, MTR-1, LSP-1, TAP-1, or PA-I protein activity associated with the cell. An agent that modulates EDIRF, MTR-1, LSP-1, TAP-1, or PA-I protein activity can be an agent as described herein, such as a nucleic acid or a protein, a naturally-occurring target molecule of an EDIRF, MTR-1, LSP-1, TAP-1, or PA-I protein, a peptide, an EDIRF, MTR-1, LSP-1, TAP-1, or PA-I peptidomimetic, or other small molecule. In one embodiment, the agent stimulates one or more EDIRF, MTR-1, LSP-1, TAP-1, or PA-I protein activities. Examples of such stimulatory agents include active EDIRF, MTR-1, LSP-1, TAP-1, or PA-I protein and a nucleic acid molecule encoding EDIRF, MTR-1, LSP-1, TAP-1, or PA-I that has been introduced into the cell. In another embodiment, the agent inhibits one or more EDIRF, MTR-1, LSP-1, TAP-1, or PA-I protein activities. Examples of such inhibitory agents include antisense EDIRF, MTR-1, LSP-1, TAP-1, or PA-I nucleic acid molecules and anti- EDIRF, MTR-1, LSP-1, TAP-1, or PA-I antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of an EDIRF, MTR-1, LSP-1, TAP-1, or PA-I protein or nucleic acid molecule. In one embodiment, the method involves administering an agent (e.g., an agent identified by a screening assay described herein), or combination of agents that modulates (e.g., upregulates or downregulates) EDIRF, MTR-1, LSP-1, TAP-1, or PA-I expression or activity. In another embodiment, the method involves administering an EDIRF, MTR-1, LSP-1, TAP-1, or PA-I protein or nucleic acid molecule as therapy to compensate for reduced or aberrant EDIRF, MTR-1, LSP-1, TAP-1, or PA-I expression or activity.

Stimulation of EDIRF, MTR-1, LSP-1, TAP-1, or PA-I activity is desirable in situations in which EDIRF, MTR-1, LSP-1, TAP-1, or PA-I is abnormally downregulated and/or in which increased EDIRF, MTR-1, LSP-1, TAP-1, or PA-I activity is likely to have a beneficial effect. One example of such a situation is where a subject has a disorder characterized by aberrant immune responsiveness. Another example of such a situation is where the subject has a inflammatory disease (e.g., arthritis or psoriasis). Yet another example of such a situation is where a subject has a disorder characterized by an aberrant hematopoietic response. Yet another example of such a situation is where a-subject has a disorder characterized by aberrant differentiation or development.

3. Pharmacogenomics

The EDIRF, MTR-1, LSP-1, TAP-1, or PA-I molecules of the present invention, as well as agents, or modulators which have a stimulatory or inhibitory effect on EDIRF, MTR-1, LSP-1, TAP-1, or PA-I activity (e.g., EDIRF, MTR-1, LSP-1, TAP-1, or PA-I gene expression) as identified by a screening assay described herein can be administered to individuals to treat (prophylactically or therapeutically) disorders (e.g., immune response disorders or developmental disorders) associated with aberrant EDIRF, MTR-1, LSP-1, TAP-1, or PA-I activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an EDIRF, MTR-1, LSP-1, TAP-1, or PA-I molecule or EDIRF, MTR-1, LSP-1, TAP-1, or PA-I modulator as well as tailoring the dosage and/or therapeutic regimen of treatment with an EDIRF, MTR-1, LSP-1, TAP-1, or PA-I molecule or EDIRF, MTR-1, LSP-1, TAP-1, or PA-I modulator.

Pharmacogenomics deals with clinically significant hereditary variations in the response to drugs due to altered drug disposition and abnormal action in affected persons. See e.g., Eichelbaum, M., Clin Exp. Pharmacol. Physiol, 1996, 23(10-11) :983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an EDIRF, MTR-1, LSP-1, TAP-1, or PA-I protein or EDIRF, MTR-1, LSP-1, TAP-1, or PA-I receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an EDIRF, MTR-1, LSP-1, TAP-1, or PA-I molecule or EDIRF, MTR-1, LSP-1, TAP-1, or PA-I modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an EDIRF, MTR-1, LSP-1, TAP-1, or PA-I molecule or EDIRF, MTR-1, LSP-1, TAP-1, or PA-I modulator, such as a modulator identified by one of the exemplary screening assays described herein.

This invention is further illustrated by the following examples which should not be construed as limiting. The contents of all references, patents and published patent applications cited throughout this application are hereby incorporated by reference.

### EXAMPLES

### Example 1

### Isolation and Characterization of Human EDIRF cDNAs

In this example, the isolation and characterization of the gene encoding human EDIRF (also referred to as “TANGO 89”) is described.

Isolation of a First Human EDIRF cDNA (Human EDIRF I), Clone jthua012a05

A human EDIRF cDNA was isolated from a human fetal lung cDNA library. To construct the library, three micrograms of poly A⁺ RNA were isolated from human fetal lung tissue, reverse transcribed, and used to synthesize a cDNA library using the Superscript cDNA Synthesis kit™ (Gibco BRL; Gaithersburg, Md.). Complementary DNA was directionally cloned into the expression plasmid pMET7 using the SalI and NotI sites in the polylinker to construct a plasmid library. Transformants were picked and amplified for single-pass sequencing. Additionally, human fetal lung cDNA was ligated into the SalI/NotI sites of the ZIPLOX™ vector (Gibco BRL) for construction of a lambda phage cDNA library.

Clone jthua012a05 was identified from the above-described cDNA library using the following method. First, each sequence was checked to determine if it was a bacterial, ribosomal, or mitochondrial contaminant. Such sequences were excluded from the subsequent analysis. Second, sequence artifacts, such as vector and repetitive elements, were masked and/or removed from each sequence. Third, the remaining sequences were searched against a copy of the GenBank nucleotide database using the BLASTN™ program (BLASTN 1.3MP: Altschul et al., J. Mol. Bio. 215:403, 1990). Fourth, the sequences were analyzed against a non-redundant protein database with the BLASTX™ program, which translates a nucleic acid sequence in all six frames and compares it against available protein databases (BLASTX 1.3MP:Altschul et al., supra). This protein database is a combination of the Swiss-Prot, PIR, and NCBI GenPept protein databases.

The original first pass sequence of clone jthua012a05 showed homology to interleukin-17 using the BLASTX™ program. The nucleotide sequence and predicted amino acid sequence are shown in FIG. 1A (corresponding to SEQ ID NO:1 and SEQ ID NO:2, respectively.) The COOH-terminus of this human EDIRF protein (corresponding to amino acids 95-180 of the predicted amino acid sequence) shows 41% identity with IL-17 (see FIG. 3). This human EDIRF protein contains a signal sequence from amino acids 1-22.

A BLASTN™ search of the EST database revealed the following ESTs having significant homology to clone jthua012a05:

| | | Base Pairs | % | |
|---|---|---|---|---|
| EST Database hits | Species | Covered | Identity | Coding? |
| Accession # AA033733 | human | 60-511 | 99 | yes |
| Accession # AA443286 | human | 718-321 | 99 | yes |
| Accession # AA680405 | human | 706-310 | 99 | yes |
| Accession # W74664 | human | 713-259 | 91 | yes |
| Accession # AA044549 | mouse | 20-517 | 87 | yes |
| Accession # W74558 | human | 39-411 | 99 | yes |
| Accession # W87101 | mouse | 60-489 | 78 | yes |
| Accession # W89980 | mouse | 90-504 | 76 | yes |
| Accession # W83241 | mouse | 12-497 | 78 | yes |

Isolation of the Murine EDIRF cDNA (Murine EDIRF I)

The gene encoding human IL-17 was initially used to conduct a database search of published expressed sequence tag (EST) sequences using the BLAST program TBLASTN (Washington University version 2.0). All matched sequences with a score of 70 or better (BLOSUM62 substitution matrix, default gap penalties of −10 for insertion and −10 for extension, no pre-filtering of the query sequence) were selected for further analysis. Matched sequences were utilized to search a database of known proteins using BLASTX (parameters were set the same as for the TBLASTN search described above). Sequences corresponding to known proteins (those having from 90-100% amino acid identity to the query matched sequence) were discarded from the analysis. Five mouse EST sequences were identified in the EST database (Accession Nos.: AA015563, W83241, W87101, AA044549, and W89980). Each sequence had been deposited by Washington University as part of the WashU-HHMI Mouse EST project and the corresponding clones were available through the Research Genetics IMAGE Consortium (IMAGE clone #468019, #405946, #407352, #475876, and #407493, respectively). Clone #407352 was subsequently amplified and fully sequenced. The full-length nucleotide sequence of murine EDIRF is presented in FIG. 2 in addition to the predicted amino acid sequence. The nucleotide and amino acid sequences of FIG. 2 correspond to those of SEQ ID NO:3 and SEQ ID NO:4, respectively.

The COOH-terminus of murine EDIRF (corresponding to amino acids 95-180 of the predicted amino acid sequence) shows 38% identity with IL-17 (see FIG. 3). This murine EDIRF protein contains a signal sequence from amino acids 1-22.

Isolation of a Second Human EDIRF cDNA (Human EDIRF II)

In addition to the five murine EST sequences described above, two additional human nucleotide sequences were published in the EST database (Accession Nos.: W74664 and W74558) which had a significant degree of homology to human IL-17. The sequences had been deposited by Washington University as part of the WashU-Merck Mouse EST project and the corresponding clone was available through the Research Genetics IMAGE Consortium (IMAGE clone #344649). When this clone was isolated and fully sequenced, however, it showed no homology to either the database entries or to the known sequence for IL-17. Four oligonucleotides were therefore designed from the published human EST sequence (oDH53, oDH54, oDH55 and oDH56). These oligos were used as PCR primers in an attempt to amplify corresponding cDNA sequences from several libraries selected a potential sources of human EDIRF sequences. Two oligos were used as forward primers: oDH53 and oDH54, and two as reverse primers: oDH55 and oDH56.

oDH53 5′ GCC CAG CTG AGG AAC AGT 3′ (SEQ ID NO:17)

oDH54 5′ GAG CTG GCC CAG AGA AAG 3′ (SEQ ID NO:18)

oDH55 5′ ACA GGA ACC TGG CTG AAC 3′ (SEQ ID NO:19)

oDH56 5′ AGC CCA CAG CGA TGG TCT 3′ (SEQ ID NO:20)

Two microliters of each of the following cDNA libraries were used in the PCR amplification reactions:

1. Clontech™ lung (lambda gt10)

2. ZIPLOX™ fetal lung*

3. ZIPLOX bronchial epithelial cell*

4. Clontech™ T Cell (lambda gt11)

5. Clontech™ Jurkat (lambda gt11)

6. Clontech™ lymphocyte (lambda gt11)

7. Stratagene™ lung (lambda ZAP™)

8. ZIPLOX™ HL60 stimulated cell line*

9. ZIPLOX™ HUVEC*

10. ZIPLOX™ HMVEC*

11. monocyte (lambda ZAP™)*

*ZIPLOX™ cDNA libraries were constructed utilizing mRNA isolated from the respective cell or tissue sources using the ZIPLOX™ cDNA library construction kit (Gibco BRL)

Each possible primer combination was used with the following PCR profile:

94° C.-4′ followed by

94° C. -30″

55° C. -30″

72° C. -30″

for 30 cycles.

Expected size of amplification products:

| | |
|---|---|
| oDH53 & oDH55 | 240 bp |
| oDH53 & oDH56 | 320 bp |
| oDH54 & oDH55 | 220 bp |
| oDH54 & oDH56 | 300 bp |

Various nucleic acid fragments were identified by ethidium bromide staining of PCR products electrophoresed through agarose gels, however, none were or the predicted size. Using the primer combination oDH53 and oDH56, there were distinctly-sized PCR products from three of the cDNA libraries: ZIPLOX™ fetal lung, ZIPLOX™ bronchial epithelial cell, and Stratagene™ lung. The PCR products from each library were identical and were 220 bp, 400 bp, and 600 bp in size.

These reaction products were each purified using standard conditions detailed in the Qiagen QIAquick PCR Purification kit™. Two microliters of the purified reaction products were used in a second PCR reaction with the primers oDH54 & oDH55 (both interior to oDH53 & oDH56). The secondary reaction performed utilizing the primary PCR product from the ZIPLOX™ fetal lung library was of the predicted size (220 bp).

This 220 bp fragment was excised from the gel and the PCR product sequenced. In addition it was subcloned using Invitrogen's TA Cloning kit™. The nucleotide sequence of the PCR product had homology to the published human EST sequences. It was therefore utilized to isolate additional clones in an attempt to obtain the full-length sequence of this second human EDIRF cDNA.

The 220 bp fragment was labeled using Stratagene's Prime-It RT kit™ and hybridized with approximately 10⁶ clones of the ZIPLOX™ fetal lung library, plated on 20 NZY plates. Eight primary positive clones were identified in the first round of screening. These were eluted and replated on fresh plates, and five of the eight showed strong positive hybridization signals in the secondary screening. Isolated plaques from the positive phage were then eluted (four of the five positives required tertiary purification), an aliquot of each (25 microliters) was combined with 100 microliters DH10B ZIP™ cells to excise the pZL1 plasmid. One of the resulting bacterial colonies was amplified and plasmid DNA was purified by Qiagen™ mini-prep kit and digested with SalI and NotI to determine the insert size. The 1.5 kB insert was ultimately sequenced and the nucleotide sequence is shown in FIG. 1B (the nucleotide sequence corresponds to SEQ ID NO:5). This nucleotide sequence corresponds to human EDIRF II which has significant homology to human EDIRF I and includes additional 5′ nucleotide sequence not present in human EDIRF I. The predicted amino acid sequence of human EDIRF II is also shown in FIG. 1B (corresponding to SEQ ID NO:6). The nucleotide sequence contains an open reading frame from nucleotide 309-911, encoding a predicted protein of 261 amino acids. The COOH-terminus of this human EDIRF protein (corresponding to amino acids 176-261 of the predicted amino acid sequence) shows 41% identity with IL-17 (see FIG. 3).

### Example 2

Distribution of EDIRF mRNA in Human and Murine Tissues

Northern Blot Analysis

The expression of EDIRF was analyzed using Northern blot hybridization. For analysis of human EDIRF, two primers pDH140RP01 [5′ TTA CCA TTT CCA TCT TCC TGG 3′] (SEQ ID NO:10) and pDH140FP01 [5′ TGC AGG TGC AGC CCA CAG 3′] (SEQ ID NO:11) were used to amplify a 504 base pair (bp) fragment for use as a probe (corresponding to nucleotides 100-603 of SEQ ID NO:1 and to nucleotides 403-906 of SEQ ID NO:5). For murine EDIRF, a vector primer adjacent to the insert was used in conjunction with primer pDH93FP01 [5′ GCA TGG TGA AGG GAT TCA CGC 3′] (SEQ ID NO:12) to amplify a 491 bp fragment covering 470 nucleotides of the 5′ end of the EDIRF gene (corresponding to nucleotides 448 through 756 of SEQ ID NO:3). The probe DNAs were radioactively labeled with ³²P-dCTP using the Prime-It kit™ (Stratagene, La Jolla, Calif.) according to the instructions of the supplier. Filters containing mRNA (human MTNI and MTNII and murine embryo MTN from Clontech, Palo Alto, Calif.; ) were probed in ExpressHyb™ hybridization solution (Clontech) and washed at high stringency according to manufacturer's recommendations.

Low level expression of an approximately 1.0 kb transcript was observed in murine adult lung. In addition, a high molecular weight signal (hybridization between 4.4 and 10 kb) was observed in adult murine brain and, to a lower level, in skeletal muscle which is believed to be unrelated to the 1.0 kb EDIRF signal. On a Northern blot of fetal mRNA, a developmentally regulated transcript of ˜1.0 kb was observed. The signal peaked in mRNA isolated from day 15 embryos, was significantly lower at both day 11 and day 17, and was absent at day 7. No other hybridization signal was observed (see below for in situ hybridization results.)

The mRNA expression of human EDIRF differed from that observed for murine EDIRF. Varying levels of two bands at ˜5.0 kb and <1 kb were observed in all tissues (spleen, thymus, prostate, testis, ovary, small intestine, colon, peripheral blood leukocytes, heart, brain, placenta, lung, liver, skeletal muscle, kidney and pancreas.) In addition, a faint transcript at ˜1.0 kb was seen in testis, heart, and liver. Furthermore, the Northern blots of adult mouse and human mRNAs took significantly longer to generate an exposure for, suggesting that the highest levels of this transcript are embryonic.

In situ Hybridization Analysis

For in situ hybridization analysis of murine EDIRF, 10 μm sagittal sections of fresh frozen embryonic day 13.5 and postnatal day 1.5 B6 mice, as well as 8 μm cross sections of adult B6 mouse lung and heart tissues were used for hybridization. Sections were postfixed with 4% formaldehyde in DEPC-treated 1×phosphate-buffered saline at room temperature for 10 minutes before being rinsed twice in DEPC-treated 1×phosphate-buffered saline and once in 0.1 M triethanolamine-HCl (pH8.0). Following incubation in 0.25% acetic anhydride-0. 1 M triethanolamine-HCl for 10 minutes, sections were rinsed in DEPC-treated 2×SSC (1×SSC is 0.15M NaCl plus 0.015M sodium citrate). Tissue was dehydrated through a series of ethanol washes, incubated in 100% chloroform for 5 minutes, and then rinsed in 100% ethanol for 1 minute and 95% ethanol for 1 minute and allowed to air dry.

The hybridization was performed using a ³⁵S-radiolabeled cRNA probe from the following DNA sequence (corresponding to nucleotides 246-510 of SEQ ID NO:3):

GAATGGAAGAGTATGAGCGGAACCTTGGGGAGATGGTGGCCCAGCTGAGGAACAGCTCCGAGCCAGCCAAGAAGAAATGTGAAGTCAATCTACAGCTGTGGTTGTCCAACAAGAGGAGCCTGTCCCCATGGGGCTACAGCATCAACCACGACCCCAGCCGCATCCCTGCGGACTTGCCCGAGGCGCGGTGCCTATGTTTGGGTTGCGTGAATCCCTTCACCATGCAGGAGGACCGTAGCATGGTGAGCGTGCCAGTGTTCAGCCA (SEQ ID NO:13)

Tissues were incubated with probe (approximately 5×10⁷ cpm/ml) in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecyl sulfate (SDS), and 0.1% sodium thiosulfate for 18 h at 55° C. After hybridization, slides were washed with 2×SSC. Sections were then sequentially incubated at 37° C. in TNE (a solution containing 10 mM Tris-HCl (pH 7.6), 500 mM NaCl, and 1 mM EDTA), for 10 minutes, in TNE with 10 ug of RNase A per ml for 30 minutes, and finally in TNE for 10 minutes. Slides were then rinsed with 2×SSC at room temp, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR™ scientific imaging film for 6 days at room temperature.

Following a 6 day film exposure signal could be seen only in the embryonic day 13.5 sections. Signal was apparent in the diaphragm and skeletal/cartilage structure or muscle structure throughout the embryo, and was readily observed in the developing spinal column and sternum. Signal was also observed at similar intensity in the tongue, developing jaw area, tissue surrounding the eye, tissue along the back of the neck, a thin layer just below the skin throughout most of the embryo, and in the developing appendages, all in a pattern similar to that of muscle. Signal was not observed in the developing cranium or large bone of the legs. This absence of signal suggests that the EDIRF signal most likely originated from developing muscle rather that bone or cartilage.

No signal was seen in the postnatal day 1.5 or adult lung and heart tissue sections. The postnatal day 1.5 tissue sections contain similar structures as the embryonic day 13.5 sections (diaphragm, sternum, spinal column, etc.) leading to the conclusion that EDIRF expression drops below detectable limits between embryonic day 13.5 and postnatal day 1.5, consistent with the results obtained from the analysis of embryonic expression using the Northern blots.

### Example 3

Characterization of Murine EDIRF I Epitope-Tagged Protein

Expression of Murine EDIRF I:flag

A murine EDIRF I flag epitope-tagged protein (mEDIRF:flag) vector was constructed by PCR followed by ligation into an expression vector, pMET stop. The full-length open-reading frame was PCR amplified using a 5′ primer incorporating a Kozak sequence upstream of and including the initiator methionine and a 3′ primer including the nucleotide sequence encoding the DYKDDDDK flag epitope (SEQ ID NO:21) followed by a termination codon. The primer sequences are shown below:

| | |
|---|---|
| 5′ primer (muT89F): | |
| 5′ AAA AAA GAA TTC GCC GCC ATG GAC | (SEQ ID NO:22) |
| TGG CCG CAC 3′ | |
| 3′ primer (muT89RF): | |
| 5′ TCC TCT GTC GAC TCA CTT GTC GTC | (SEQ ID NO:23) |
| GTC GTC CTT GTA GTC GAA GAT GCA GGT | |
| GCA 3′ | |

The sequenced DNA construct was transiently transfected into HEK 293T cells in 150 mM plates using Lipofectamine (GIBCO/BRL, Gaithersburg Md.) according to the manufacturer's protocol. Seventy-two hours post-transfection, the serum-free conditioned medium (OptiMEM, GIBCO/BRL) was harvested and spun.

N-terminal Sequencing of mEDIRF:flag

The 293 conditioned medium was electroblotted onto a PVDF membrane (Novex) after separation by SDS-PAGE on 4-20% gradient gels and probed with M2 anti-flag polyclonal antibody diluted 1:500 (Sigma Chemical Co., St. Louis Mo.) followed by horseradish peroxidase conjugated sheep anti-mouse antibody diluted 1:5000 (Amersham Pharmacia Biotech, Inc., Piscataway N.J.) developed with chemiluminescent reagents (Renaissance, DuPont-NEN Research, Boston Mass.) and exposed to autoradiography film (BioMax MR2 film, Eastman Kodak Co., Rochester, N.Y.). Flag immunoreactivity appeared as two pairs of bands that migrated on SDS-PAGE between the 17 and 30 kDa markers (Multimark, Novex).

Samples of each of the four mEDIRF:flag bands that were bound to PVDF membrane after SDS-PAGE and electroblotting were subjected to N-terminal amino acid sequence analysis using Edman-based chemistry protein sequencing on a PE Applied Biosystems Model 494 sequencer (Applied Biosystems, Inc., Foster City Calif.). The amino acid residues were analyzed by HPLC using a Spherogel micro PTH 3-micron column and identified by separation and peak height as compared to standards. As shown below in Table 1, one band (band C) gave no interpretable signal and the remaining three have the same N-terminal sequence. As all these bands share the same N-terminus and the same C-terminus (flag), the primary structure of the different molecular weight bands is likely to be the same. The different forms may arise through non-proteolytic post-translational modification, for example, by differences in N-glycosylation at a predicted N-glycosylation site (Asn75 of SEQ ID NO:3). The sequence obtained confirms the deduced amino acid sequence encompassing amino acids 23 to 33 shown in FIG. 2.

Determination of mEDIRF:flag Protein Molecular Weight

Nine hundred fifty mL of the 293 conditioned medium was passed over a sulphopropyl cation exchange resin (Poros HS, PE Biosystems Division of The Perkin-Elmer Corp., Norwalk Conn.) equilibrated in 20 mM MES pH 6.0 and eluted with a gradient of 0-1.0 M NaCl in the same buffer. Samples of unbound material and fractions collected during the elution were analyzed by silver stained SDS-PAGE and SDS-PAGE/flag western blot as above. No mEDIRF:flag immunoreactive material was detected in the unbound sample, whereas intense immunoreactivity was observed in fractions eluted over the gradient corresponding to between 0.55M and 0.8M NaCl. The molecular weight of each of the four bands on silver-stained SDS-PAGE were measured relative to protein molecular weight standards. The calculated molecular weights in kDa are summarized below in Table 1.

TABLE 1
N-terminal sequence and MW of mEDIRF:flag

| | N-terminal sequence | | Molecular weight |
|---|---|---|---|
| band A | RNTKGKRKGQG | (SEQ ID NO:59) | 25 |
| band B | RNTKGKRKGQG | (SEQ ID NO:59) | 23 |
| band C | Not determined | 20 | |
| band D | RNTKGKRKGQG | (SEQ ID NO:59) | 19 |

Because the purity of cation exchange purified mEDIRF:flag was less than 50%, a second purification step was employed to eliminate non-immunoreactive contaminants. mEDIRF:flag fractions obtained by cation exchange were pooled, then fractionated over a quaternized polyethyleneimine anion exchange resin (Poros HQ, PE Biosystems) equilibrated in 20 mM Tris pH 8.0 and eluted over a gradient of 0-1.0 M NaCl in the same buffer. Samples of unbound material and of fractions collected during the elution were analyzed by silver stained SDS-PAGE and SDS-PAGE/flag western blot as above. No mEDIRF:flag immunoreactive material was detected in the unbound sample, whereas intense immunoreactivity was observed in fractions eluted over the gradient corresponding to between 0.3M and 0.4M NaCl. The purity of mEDIRF:flag after sequential cation and anion exchange chromatography was more than 90% as judged by silver stained SDS-PAGE.

### Example 4

Localization of Murine EDIRF:flag

To analyze cell-associated mEDIRF:flag, HEK 293T cells were transfected as above in multiple wells of 6-well tissue culture plates. After 72 hours, cells were washed with PBS and then incubated with 0.05% trypsin in Hank's medium or in a PBS control. After 0, 3 and 20 minutes, wells were scraped and cells pelleted by centrifugation. Cell pellets were boiled in a reducing SDS-PAGE sample buffer and analyzed by SDS-PAGE/flag western blot. The PBS control cell pellets contained significant mEDIRF:flag immunoreactivity. Cells treated with trypsin for 3 or 20 minutes exhibited more than 3-fold reduction in immunoreactivity, suggesting that most of cell-associated murine EDIRF is exposed to trypsin and thus on the cell surface rather than within the plasma membrane.

To analyze the release of mEDIRF:flag from the cell surface, HEK 293 cells were transfected as above in multiple wells of 6-well tissue culture plates. After 72 hours, cells were washed with PBS and then incubated with heparin (1 mg/mL or 0.01 mg/mL) or PBS as control. After 60 minutes at 4° C., supernatants and cells were harvested by centrifugation and boiled in SDS-PAGE sample buffer. SDS-PAGE/flag western blot analysis showed that addition of soluble heparin results in an increase in mEDIRF:flag immunoreactivity in the supernatant and a corresponding decrease in the pellet relative to controls. Both concentrations of heparin were equally effective. These data suggest that mEDIRF:flag is associated with the surface of the cell through association with sulfated proteoglycan, probably heparin. Thus, EDIRF is predicted to be cell-associated, suggesting that EDIRF may function primarily as a localized modulator.

### Example 5

Binding of EDIRF I to Mouse Tissues

Preparation of AP:EDIRF I

A mouse EDIRF I alkaline phosphatase N-terminal fusion protein (AP:mEDIRF) vector was constructed by PCR followed by ligation into the AP-Tag3 vector (Tartaglia, L., et al., 1995. Cell 83:1263-1271). The full-length open-reading frame of murine EDIRF I was PCR amplified using a 5′ primer incorporating a BglII restriction site prior to the nucleotides encoding the first amino acids of the mature murine EDIRF I protein (RNTK; SEQ ID NO:30) and a 3′ primer including a XhoI restriction site immediately following the termination codon of murine EDIRF I. Thus the open reading frame of the complete construct includes the complete sequence of human placental alkaline phosphatase, including the signal peptide, followed by the mature murine EDIRF I DNA sequence. The primer sequences are shown below:

| | |
|---|---|
| 5′ primer: | |
| 5′ TTT TTA GAT CTC GGA ACA CCA AAG | (SEQ ID NO:24) |
| GCA AAA G 3′ | |
| 3′ primer: | |
| 5′ TTT TGT CGA CTC AGA AGA TGC AGG | (SEQ ID NO:25) |
| TGC AAC 3′ | |

The sequenced DNA constructs were transiently transfected into HEK 293T cells in 150 mM plates using Lipofectamine (GIBCO/BRL) according to the manufacturer's protocol. Seventy-two hours post-transfection, the serun-free conditioned media (OptiMEM, GIBCO/BRL) were harvested, spun and filtered. Alkaline phosphatase activity in conditioned media was quantitated using an enzymatic assay kit (Phospha-Light, Tropix Inc.) according to the manufacturer's instructions.

When alkaline phosphatase fusion protein concentrations below 2 nM were observed, conditioned medium was concentrated by centrifugation using a 30 kDa cut-off membrane. Conditioned medium samples before and after concentration were analyzed by SDS-PAGE followed by Western blot using anti-human alkaline phosphatase antibodies diluted 1:250 (Genzyme Corp., Cambridge Mass.) and detected by chemiluminescence. A band at 90 kDa was observed in concentrated supernatants of AP:mEDIRF transfections. Conditioned medium samples were adjusted to 10% fetal calf serum and stored at 4° C.

Screening of Tissue Sections for AP:mEDIRF Binding

Supernatant containing AP:mEDIRF was used to screen tissue sections essentially as described by Cheng and Flanagan (1994) Cell 79:157-168. Briefly, fresh frozen sections (8 μm) were prepared and rinsed in HBHA (Hank's balanced salt solution supplemented with 20 mM HEPES, pH 7.0, 0.05% BSA, 0.1% sodium azide). Tissue sections were subsequently incubated with supernatant containing AP:mEDIRF or alkaline phosphatase at a concentration of 5 nM for one hour at room temperature. After the incubation, tissue sections were washed six times in HBHA, fixed (60% acetone, 3% formaldehyde, 20 mM HEPES, pH 7.5), washed three times in HBS (20 mM HEPES, pH 7.5, 150 mM NaCl), then heated for 30 min at 65° C. to inactivate endogenous alkaline phosphatase activity. Bound AP:mEDIRF was detected by developing sections in BCIP/NBT substrate solution (100 mM Tris-HCl, pH 9.5, 100 mM NaCl, 5 mM MgCl, 0.17 mg/ml BCIP and 0.33 mg/ml NBT).

Using this procedure, tissue sections of mouse embryos (day 14.5 of prenatal development) and newborn mice (postnatal day 1.5) were screened for EDIRF I binding. AP:mEDIRF but not alkaline phosphatase alone bound strongly to all tissue structures. In the presence of increasing concentrations of heparin (30 U-1000 U/ml), the overall binding of AP:mEDIRF generally decreased, although binding of AP:mEDIRF to certain structures such as fetal liver, skeletal muscle and cartilage of the embryo sections was retained. This suggests that EDIRF I may be binding to heparin or to heparin-like molecules in the majority of fetal and newborn mouse tissues. The binding characteristics of a human EDIRF I alkaline phosphatase N-terminal fusion protein were similar to AP:mEDIRF.

### Example 6

Expression of Human EDIRF I

Construction of a Human EDIRF I Expression Vector

Human EDIRF I was PCR amplified from jthua012a05 using the following 5′ and 3′ PCR primers:

| | |
|---|---|
| 5′ (B91): | |
| GATCGATCGATCCCATGGACTGGCCTCACAA | (SEQ ID NO:26) |
| 3′ (B92): | |
| TGACTGACTGACTGTCGACTCACTACTTGTCATCGT | (SEQ ID NO:27) |
| CATCCTTATAGTCGAAGATGCAGGTGCAG | |

The resulting PCR product encoded an NCO I site in frame with the initiating MET of human EDIRF at the 5′ end and a fused flag tag (GACTATAAGGATGACGATGACAAG) (SEQ ID NO:28) followed by two stop codons (TAGTGA) and a HindlIl site at the 3′ end. The resulting product is as follows:

| +1 | M D W P H N |
|---|---|
| | NcoI |
| 1 | GATCGATCGA TCTCTAGAGC CACCATGGAC TGGCCTCACA |
| | CTAGCTAGCT AGAGATCTCG GTGGTACCTG ACCGGAGTGT |
| | L L F |
| | ACCTGCTGTT |
| | TGGACGACAA |
| +1 | L L T I S I F L G L G Q P |
| 51 | TCTTCTTACC ATTTCCATCT TCCTGGGGCT GGGCCAGCCC |
| | AGAAGAATGG TAAAGGTAGA AGGACCCCGA CCCGGTCGGG |

R S P
AGGAGCCCCA
TCCTCGGGGT

| +1 | K S K R K G Q G R P G P L A |
|---|---|
| 101 | AAAGCAAGAG GAAGGGGCAA GGGCGGCCTG GGCCCCTGGC |
| | TTTCGTTCTC CTTCCCCGTT CCCGCCGGAC CCGGGGACCG |
| | P G P |
| | CCCTGGCCCT |
| | GGGACCGGGA |
| +1 | H Q V P L D L V S R M K P Y |
| 151 | CACCAGGTGC CACTGGACCT GGTGTCACGG ATGAAACCGT |
| | GTGGTCCACG GTGACCTGGA CCACAGTGCC TACTTTGGCA |
| | A R M |
| | ATGCCCGCAT |
| | TACGGGCGTA |
| +1 | E E Y E R N I E E M V A Q |
| 201 | GGAGGAGTAT GAGAGGAACA TCGAGGAGAT GGTGGCCCAG |
| | CCTCCTCATA CTCTCCTTGT AGCTCCTCTA CCACCGGGTC |
| | L R N |
| | CTGAGGAACA |
| | GACTCCTTGT |
| +1 | S S E L A Q R K C E V N L Q |
| 251 | GCTCAGAGCT GGCCCAGAGA AAGTGTGAGG TCAACTTGCA |
| | CGAGTCTCGA CCGGGTCTCT TTCACACTCC AGTTGAACGT |
| | L W M |
| | GCTGTGGATG |
| | CGACACCTAC |
| +1 | S N K R S L S P W G Y S I N |
| 301 | TCCAACAAGA GGAGCCTGTC TCCCTGGGGC TACAGCATCA |
| | AGGTTGTTCT CCTCGGACAG AGGGACCCCG ATGTCGTAGT |
| | H D P |
| | ACCACGACCC |
| | TGGTGCTGGG |
| +1 | S R I P V D L P E A R C L |
| 351 | CAGCCGTATC CCCGTGGACC TGCCGGAGGC ACGGTGCCTG |
| | GTCGGCATAG GGGCACCTGG ACGGCCTCCG TGCCACGGAC |
| | C L G |
| | TGTCTGGGCT |
| | ACAGACCCGA |
| +1 | C V N P F T M Q E D R S M V |
| 401 | GTGTGAACCC CTTCACCATG CAGGAGGACC GCAGCATGGT |
| | CACACTTGGG GAAGTGGTAC GTCCTCCTGG CGTCGTACCA |
| | S V P |
| | GAGCGTGCCG |
| | CTCGCACGGC |
| +1 | V F S Q V P V R R R L C P P |
| 451 | GTGTTCAGCC AGGTTCCTGT GCGCCGCCGC CTCTGCCCGC |
| | CACAAGTCGG TCCAAGGACA CGCGGCGGCG GAGACGGGCG |
| | P P R |
| | CACCGCCCCG |
| | GTGGCGGGGC |
| +1 | T C P C R Q R A V M E T I |
| 501 | CACAGGGCCT TGCCGCCAGC GCGCAGTCAT GGAGACCATC |
| | GTGTCCCGGA ACGGCGGTCG CGCGTCAGTA CCTCTGGTAG |
| | A V C |
| | GCTGTGGGCT |
| | CGACACCCGA |
| +1 | C T C I F D Y K D D D D K * |
| | * |
| | HindIII |
| | ˜˜˜˜˜˜ |
| 551 | GCACCTGCAT CTTCGACTAT AAGGATGACG ATGACAAGTA |
| | CGTGGACGTA GAAGCTGATA TTCCTACTGC TACTGTTCAT |
| | GTGAAAGCTT |
| | CACTTTCGAA |
| 601 | AGCTAGCTA |
| | TCGATCGAT |

The top strand of this PCR product is set forth above as SEQ ID NO:31. The bottom strand is set forth as SEQ ID NO:32 (when read in the 5′-3′ direction). The amino acid sequence encoded by this PCR product is set forth as SEQ ID NO:33. This PCR product was cut with NCO I and HindIII and cloned into pEF/myc/cyto (a pShooter™ vector from Invitrogen Corp.) cut with the same restriction enzymes. The resulting plasmid, pEF_IL 17h, was grown in ElectroMAX DH10B™ competent cells (GIBCO/BRL), and purified using the QIAFilter system (QIAGEN, Inc.)

Expression of Human EDIRF I

293T cells were grown to 80% confluency, then split into T75 flasks at 6×10⁶ cells per flask in DMEM/F12, 10%FBS culture media and incubated overnight at 37° C. Next day, the cells were washed twice with 1×PBS and DMEM/F12 supplemented with 10 mM HEPES, Insulin-Transferrin-Selenium-X supplement (GIBCO), and 2 mM glutamine. After one hour incubation at 37° C. in the serum-free medium, the cultures were transfected with pEF_IL17h in FuGENE™ 6 Transfection Reagent (Boehringer Mannheim Corp., Indianapolis Ind.). The FuGENE/DNA suspension, per T75 flask, was prepared as follows: 45 μl FuGENE was added to 750 μl DMEM/F12 supplemented media, then vortexed. Eleven μg of pEF_IL17h (at a concentration of 0.5 to 1 μg/μl) was then added and incubated for 30 minutes before adding to the cells. The supernatant was harvested after 72 hours incubation.

### Example 7

### Purification of Human EDIRF I

Human EDIRF I was purified from approximately 125 ml of media pooled from three 72-hour supernatants obtained as described above.

The media was pumped over a freshly prepared fast flow S column (30 ml bed volume; Pharmacia LKB ) at a flow rate of 5 ml/min. After all of the media had been loaded, the column was washed with 50 mM Tris pH 7.5 until the absorbance at 280 nm returned to near baseline. A linear gradient from 0-1M NaCl (in 50 mM Tris pH 7.5) was started and developed over 60 min (at a flow rate of 5 ml/min. Absorbance was monitored at 280 nm and 5 ml fractions were collected. The fractions were analyzed by gel electrophoresis (4-20% SDS-PAGE) and fractions containing protein at approximately the expected size were electrophoresed (4-20% SDS-PAGE), transferred to a PVDF membrane and stained with Coomassie Blue.

### Example 8

### N-terminal Sequencing of Human EDIRF I

SDS-PAGE protein bands obtained as above were excised and sequenced on an ABI 494 sequencer (Applied Biosystems, Inc.), essentially following the procedure as described above in Example 3 for the N-terminal sequencing of murine EDIRF I from which the following sequence was determined:

RSPKSKRKGQ (SEQ ID NO:29)

The sequence obtained confirms the deduced amino acid sequence encompassing amino acids 23 to 32 shown in FIG. 1A.

### Example 9

### Effect of EDIRF on Early Development

Preparation of Murine EDIRF I RNA

The template for the murine EDIRF in vitro transcription reaction was prepared from the DNA construct for the murine EDIRF flag epitope-tagged protein as described above in Example 3 inserted in a pCS2 vector, which was then linearized using AscI. Capped RNA was synthesized using SP6 RNA polymerase from the linearized plasmid using mMESSAGE mMACHINE kit (Ambion, Austin, Tex.) according to the manufacturer's instructions. Phenol:chloroform (1:1)-extracted RNA was precipitated with isopropanol. Prior to injection into embryos, the RNA was tested for translatability in vitro in the presence of ³⁵S-methionine using a rabbit reticulocyte lysate system (Promega, Madison Wis.).

Murine EDIRF I RNA Injection into Xenopus Embryos

Xenopus embryos were obtained by in vitro fertilization, dejellied in 2% cysteine HCl (pH 7.6), washed thoroughly in Modified Ringers solution, and incubated at 15-25° C. Embryos were transferred to injection solution (Modified Ringers solution containing 5% Ficoll) prior to injections. 100-500 pg of murine EDIRF I RNA was injected into each blastomere at the 2-cell stage. Embryos were transferred to water from the injection solution after approximately 6 hours and grown until the appropriate stage.

Examination two days later of embryos injected with Tango 89 RNA showed an overexpression phenotype. These results suggest that EDIRF I has a dramatic effect on early tissue differentiation.

Analyses of Embryos for EDIRF I or EDIRF II Expression

Embryos for Western analysis are frozen and then lysed in buffer containing 50 mM TrisCl pH7.5, 0.5M urea, 2% NP40, 1 mMPMSF and 5% 2-ME. Proteins are separated by gel electrophoresis and Western blotted according to standard methods. Immunodetection of protein is carried out using a BM chemiluminescence Western blotting kit according to the manufacturer's instructions (Boehringer Mannheim). Diluted anti-EDIRF I or anti-EDIRF II rabbit antiserum will be used as primary antibody.

Embryos for histological examination are fixed in 10% Zn formalin overnight, embedded in paraffin and stained with H&E by standard procedures.

Embryos for immunohistochemistry are fixed in 100% methanol at −20° C., embedded in paraffin and sectioned at 8 um as above and processed by standard procedures using various primary antibodies and horseradish peroxidase-conjugated secondary antibodies for detection.

Xenopus Cap Assay

100-500 pg of EDIRF I or EDIRF II RNA are injected into the animal pole of each of the 2 blastomeres at the 2-cell stage. Animal caps from uninjected or injected embryos will be explanted at stage 9 and cultured in 0.1×Modified Ringers containing 0.1% BSA and 50 ug/ml gentamycin. Alternatively, uninjected animal caps will be incubated in 10-100 ng/ml of purified EDIRF I or EDIRF II protein. Animal caps are cultured until control embryos have reached stage 19-20. Poly A+ RNA is extracted from lysed animal caps and whole embryos using mAP paper (Amersham, Ill.) as described by Amaravadi and King (1994) Oncogene 9:971-974. RT-PCR is performed on these samples using gene-specific primers and appropriate annealing temperatures and the products analyzed by gel electrophoresis.

### Example 10

### Isolation and Characterization of Murine MTR-1 cDNA

In this example, the isolation and characterization of the gene encoding murine MTR-1 (also referred to as TANGO 49) is described.

Isolation of the Murine MTR-1 cDNA

Poly A+ RNA from mouse choroid plexus was used to construct a cDNA library. Total RNA was isolated from mouse choroid plexus using the guanidinium isothiocyanate/CsCl method of Chirgwin et al. (1979) Biochemistry 18:5294 (as described by R. Selden in Current Protocols for Molecular Biology). After quantitation, the RNA was dilute to 1 mg/ml in distilled deionized water and incubated for 30 minutes at 37° C. with an equal volume of DNase solution (20 mM MgCl₂, 2 mM DTT, 0.1 units DNase, 0.6 units RNase inhibitor in TE) to remove contaminating DNA. The RNA was extracted with phenol/chloroform/isoamyl, and ethanol precipitated. After quantitation at 260 nm, an aliquot was electrophoresed to check the integrity. Poly A+ RNA was isolated using Oligotex-dT™ kit from Qiagen (Chatsworth, Calif.). After quantitation the mRNA was ethanol precipitated and resuspended in at 1 mg/ml in distilled DEPC treated water. The cDNA was synthesized according to the method of Gubler and Hoffman ((1983) Gene 25:263) using a Superscript™ Plasmid cDNA synthesis kit from Life Technologies (Gaithersburg, Md.). The cDNA obtained was ligated into the NotI/SalI sites of pMET vector. Ligated cDNAs were ethanol precipitated and resuspended in distilled water. One microliter of the DNA was transformed by electroporation into electrocompetent DH10B E. coli. The insert cDNAs of a number of clones were sequenced by standard automated fluorescent dideoxynucleotide sequencing and dye primer chemistry (Applied Biosystems Inc., Foster City Calif.) on Applied Biosystems sequenators. Each sequence was checked to determine if it was bacterial, ribosomal or a mitrocondrial contaminant and sequence artifacts were removed.

### Example 11

### Distribution of MTR-1 mRNA in Mouse Tissues

Northern Blot

Clontech (Palo Alto, Calif.) Mouse Northern Blot was probed by mouse MTR-1 as recommended by the manufacturer. In mouse, two bands of approximately 3 kb and 4 kb, were found in heart, skeletal muscle and kidney.

In situ Hybridization

In situ hybridization was performed with mouse antisense MTR-1 probe on E17 (day 17) mouse embryo. Apart from the high expression in choroid plexus, signals were also detected on the membrane surrounding brain and spinal cord, the ventricles of brain, dorsal root ganglia, tongue, olfactory epithelium and developing mandible.

### Example 12

### Chromosomal Mapping of the Murine MTR-1 Gene

This Example describes the chromosomal mapping of the murine MTR-1 gene.

The MTR-1 gene was mapped utilizing a backcross panel of CB57/Spartus mice. MTR-1 is located on chromosome 10 near marker D10MIT86. This region is syntenic to 6q in human. PCR primers were used to amplify mouse genomic DNA using standard techniques. Primers were designed from noncoding sequences of murine T75 and were as follows:

| | |
|---|---|
| Forward Primer: | |
| 5′ ACTTACCACGTGTGCCTCTGAA3′ | (SEQ ID NO:41) |
| Reverse Primer: | |
| 5′ ATACAGGCCTGAGGAACAGATT3′ | (SEQ ID NO:42) |

Amplification conditions were 30 cycles at 94° C. for 30 seconds, 55° C. for 30 seconds and 72° C. for 30 seconds.

### Example 13

### Characterization of MTR-1 Proteins

In this example, the predicted amino acid sequences of the murine MTR-1 protein was compared to amino acid sequences of known proteins and various motifs were identified. In addition, the predicted hydrophobicity of the murine MTR-1 protein was predicted using the Kite-Doolittle hydropathy prediction method.

The murine MTR-1 cDNA encodes a protein of 622 amino acids (predicted MW of 67 kDa, not including post-translational modifications). There are twelve predicted transmembrane sequences (aa 44-67, aa 85-106, aa 112-130, aa 142-160, aa 170-192, aa 202-220, aa 283-304, aa 322-343, aa 351-368, aa 469-488, aa 503-523, aa 534-552).

FIG. 5 illustrates the alignment of amino acids 495-622 of murine MTR-1 (SEQ ID NO:35) with human glucose transporter 3 (SEQ ID NO:39). As demonstrated, human glucose-transporter 3 shared 32% amino acid sequence identity with murine MTR-1. In addition, FIG. 6 provides the alignment data of amino acids 7-617 of SEQ ID NO:35 and beet sugar transporter (SEQ ID NO:40). This alignment reveals a 28% sequence identity between the two proteins.

### Example 14

### Isolation and Characterization of Human LSP-1 cDNAs

In this example, the isolation and characterization of the gene encoding human LSP-1 (also referred to as “HOMEDEPO” or “TANGO 111”) is described.

Isolation of the Human LSP-1 cDNA

The following methodology takes advantage of the fact that molecules such as LSP-1 have an amino terminal signal sequence which directs certain secreted and membrane-bound proteins through the cellular secretory apparatus.

A partial LSP-1 mRNA was identified by screening of a human bone marrow cDNA library. This library was prepared using mRNA purchased from Clontech, Palo Alto (Cat. no, 6573-1). A signal trap cDNA library was prepared by ligating random primed double stranded cDNA into the expression vector, ptrAP1, resulting in fusions of cDNAs to the reporter, alkaline phosphatase (AP). DNAs from individual clones from this library were prepared by standard techniques and transfected in to human embryonic kidney fibroblasts (293T cells). After 28 hours cell supernatants were collected and assayed for AP activity.

Clones giving rise to detectable AP activity in the supernatants of transfected cells were analyzed further by DNA sequencing and the novel clones subjected to further DNA sequencing.

Once such clone, named LSP-1, was identified. The initial LSP-1 clone contained an open reading frame of 169 amino acids, (fused in-frame to the AP reporter). Blast searching of Genbank with this sequence indicated partial homology to certain immunoglobulin type proteins (illustrated more clearly in the sequence analysis of the full length clone below). A GenBank™ search of the dbEST database utilizing the sequence of this cDNA revealed several EST sequences with greater than 95% nucleotide identity to the partial cDNA.

The LSP-1 nucleic acid molecule was aligned with the FDF03 molecule (described in WO/24906) using the GAP program in the GCG software package (pam120 matrix) and a gap weight of 12 and a length weight of 4. The results showed a 45.3% identity between the two sequences (see FIG. 9).

The LSP-1 protein was aligned with the FDF03 protein (described in WO/24906) using the GAP program in the GCG software package (pam120 matrix) and a gap weight of 12 and a length weight of 4. The results showed a 51.6% identity between the two protein sequences (see FIG. 10).

Sequencing of Full Length LSP-1 cDNAs

The interesting chromosomal localization (see Example 16) of the LSP-1 genes motivated a search for clones encoding a full length LSP-1 cDNA. Searching of the Genbank database identified several EST sequences that showed a high degree of identity to the partial LSP-1 cDNA identified by signal sequence trapping.

To obtain further sequence data, three ESTs present in the IMAGE clone collection were located and subjected to further DNA sequencing. A single sequence was assembled which completed the LSP-1 open reading frame and extended a 3′ UTR and poly A tail. FIG. 8 shows the selected clones and details the relationship between the first LSP-1 clone identified by signal trapping, the IMAGE clones and the final composite sequence.

Structure of the LSP-1 Protein

The domain structure of the full length LSP-1 proteins is depicted in FIG. 9. LSP-1 contains an N-terminal signal peptide (predicted by the signal Pa algorithm), an immunoglobulin-type domain, a transmembrane domain (predicted by MEMSAT, Jones, D. T., Taylor, W. R., and Thornton, J. M. 1994 Biochemistry 33 3038-3049) and a short cytoplasmic domain. The predicted Ig domain is incomplete. However, there is precedent for Ig domains of this kind (Barclay et al., The Leucocyte Antigen Factsbook, Academic Press).

Thus, the LSP-1 protein appears to be a Type 1 membrane protein composed of a single extracellular immunoglobulin type domain, a transmembrane domain and a short cytoplasmic tail This overall topological structure appears similar to that of other leukocyte membrane proteins, notable CD1, CD3 and CD28 (Barclay et al., The Leucocyte Antigen Factsbook, Academic Press). Also of note is the presence within the predicted LSP-1 membrane spanning domain of a lysine residue. The presence of charged groups within this domain of cell surface proteins suggests that the protein may form homodimers in the cell membrane (Barclay et al., The Leucocyte Antigen Factsbook, Academic Press). Thus, LSP-1 may exist at the cell surface as a homodimer and may exert its effects in this form.

Translation Initiation

The translation of the LSP-1 sequence is shown starting from an ATG at nucleotide 1332 of SEQ ID NO:43. This ATG is in a favorable context for translation initiation (good Kozak consensus). Although another in-frame ATG is present upstream at nucleotide 1140 of SEQ ID NO:43, this is not in a favorable context for translation initiation and given the overall predicted topology of the mature protein translated from the second methionine, it is unlikely to be used.

### Example 15

### Distribution of LSP-1 mRNA in Human Tissues

Probing of northern blots purchased from Clontech revealed a 1.5 kb transcript for LSP-1 only in peripheral blood leukocytes (PBL) and not in any other tissue. Blots contained the following tissues: spleen, thymus, prostate, testis, ovary, small intestine, colon, peripheral blood leukocyte (human multiple tissue northern III, cat. no. 7767-4) spleen, lymph node, thymus, peripheral blood Leukocyte bone marrow and fetal liver (human immune system II cat. no. 7768-1). Also, a larger 4.5 kb transcript was detectable in tissues of the endocrine system (pancreas, adrenal medulla, thyroid, adrenal cortex, testis, thymus, small intestine and stomach-human endocrine blot, cat. no. 7751-1).

PCR analysis of cDNA libraries from various sources was also performed using primers and conditioned as described for chromosomal mapping of LSP-1. PCR detects lower levels of mRNA expression than northern blotting. LSP-1 mRNA was detectable in the following human cDNA libraries; placenta, fetal brain, fetal heart, fetal liver, adult heart, human umbilical vein endothelial cells, HeLa cells, fetal kidney, adult adipose tissue, adult prostate, colorectal adenocarcinoma. lymphocytes, adult lung, adult spleen, HL-60 cells (promyelocytic leukemia), human microvascular endothelial cells and fetal spleen. LSP-1 mRNA was not detectable in prostrate epithelial cells, megakaryocytes, bronchial epithelial cells or primary osteoblasts.

### Example 16

### Chromosomal Mapping of Human LSP-1 Gene

This Example describes the chromosomal mapping of the human and murine LSP-1 genes.

The LSP-1 gene maps to chromosome 7q21-q22, at 111-112 cM (using the Généthon linkage map as reference).

The clone was mapped to two difference Radiation Hybrid (RH) panels, the Stanford Human Genome Center G3 panel and the Genebridge G4 panel, using the following primers (forward: TCACTCAACCAAAACACC (SEQ ID NO:46); reverse CCAGTTCAGAAAGACC (SEQ ID NO:47)). LSP-1 was found to be linked to Genebridge G4 framework marker D7S651, at a distance of 1.7 cR (3000) and a lod score >3; and to Stanford G3 framework marker WI-7004, at a distance of 0 cR (10000) (meaning without recombination between the clone and the marker) with a lod score of 1000. Both Markers are also part of an integrated gene map (http://www.nci.bi.nlm.nih.gov/SCIENCE96/) that serve as a general reference.

The cytogenetic location for LSP-1 was inferred from the map position of close-by genes (Epo and Cytochrome P450 IIIA).

The results of the RH panel mapping place the LSP-1 gene very close to the Erythropoietin (EPO) gene (precise distance unclear due to the lack of resolution of RH mapping). Possibly within 50-100 Kb.

### Example 17

### Isolation and Characterization of Human TAP-1 cDNAs

In this example, the isolation and characterization of the genes encoding human TAP-1 is described.

Construction of Libraries

Poly A+ RNA from human prostate tissue was used to construct a cDNA library. The cDNA library was constructed by first and second strand synthesis as recommended by the manufacturer for the Gibco BRL kit SuperScript Plasmid System for cDNA Synthesis and Plasmid Cloning (Gibco/BRL; Bethesda Md.).

Isolation of a Human TAP-1 cDNA

A partial human TAP-1 cDNA, also referred to as TANGO-94, was identified by analysis of an EST database using mouse TPO sequence as a probe. A partial human clone (jthqb070d08) was obtained from a human prostate cDNA library and was subsequently fully sequenced. Clone jthqb070d08 was deposited with the American Type Culture Collection on Oct. 2, 1997 and has ATCC Accession Number 98554.

A nucleotide sequence of the isolated C-terminal domain of human TAP-1 cDNA (nucleotides 1-528 corresponding to the C-terminus and 3′ untranslated sequence) and the predicted amino acid sequence of the human TAP-1 protein (amino acids 1-86) are shown in FIG. 12 and in SEQ ID NOs:49 and 50, respectively. The amino acid sequences showed 32% identity to the C-terminal part of human TPO. The nucleotide sequence corresponding to the coding region of the human TAP-1 cDNA are nucleotides 1-258 of SEQ ID NO:49, corresponding to SEQ ID NO:51, while nucleotides 259-528 correspond to the 3′ untranslated region of the gene.

Isolation of Additional TAP-1 cDNAs

Using jthqb070d08 cDNA as a probe, additional TAP-1 clones were isolated using standard protocols. In brief, clone (jthqb070d08 cDNA) was excised from a pMET vector using SalI and NotI restriction enzymes. The excised fragment was labeled using the Prime It kit from Stratagene (La Jolla, Calif.) and then hybridized under high stringency conditions to a human fetal liver library. For high stringency conditions, hybridizations were carried out overnight at 65° C. in Church buffer. The filters were washed the next day with 2×SCC/0.1% SDS. Eight clones were isolated from human fetal liver library and submitted for sequencing. 3 out of the 8 clones contain an insert of approximately 3 kb.

### Example 18

### Distribution of TAP-1 mRNA in Human Tissues

Northern blots using clone (jthqb070d08 cDNA) were performed using standard protocols. In brief, clone (jthqb070d08 cDNA) was excised from a pMET vector using SalI and NotI restriction enzymes. The excised fragment was labeled using the Prime It kit from Stratagene (La Jolla, Calif.) and then hybridized to multi-tissue northern blots from Clontech (Palo Alto, Calif.) as recommended by the manufacturer. A strong band was detected at approximately 3 kb in human fetal liver tissues. Additional bands were detected which may be indicative of alternate spliced variants. Two other less intense bands of approximately 5 and 2 kb were detected in all tissues tested.

### Example 19

### Analysis of TAP-1 Amino Acid Sequence

The partial human TAP-1 cDNA, which is approximately 528 nucleotides in length, and which is approximately 86 amino acid residues in length. The human TAP-1 protein contains four serine-proline-threonine-rich domains. A TAP-1 serine-proline-threonine-rich domain can be found at least, for example, from about amino acids 1-20 of SEQ ID NO:50 (Gly1 to Gly20 of SEQ ID NO:50); from about amino acids 21-40 of SEQ ID NO:50 (Ile20 to Ala40 of SEQ ID NO:50); from about amino acids 41-60 of SEQ ID NO:50 (Val40 to Gly60 of SEQ ID NO:50); and from about amino acids 61-81 of SEQ ID NO:50 (Pro61 to Thr81 of SEQ ID NO:50). The human TAP-1 C-terminal domain appears to encode a secreted protein, e.g., growth factor a secreted protein which shares significant homology, about 32% homology, with the C-terminal region of human TPO.

An alignment of the human TAP-1 amino acid sequences to human TPO sequences is presented in FIG. 13. The figure depicts an alignment of the amino acid sequences of TAP-1 (corresponding to amino acids 15 to 75 of SEQ ID NO:50) and human TPO sequences (SEQ ID NO:52; Swiss-Prot™ Accession Numbers P40225, 1401246, 939627). Identical residues are indicated in the row between the TAP-1 and the TPO sequences by a single amino acid code; conserved amino acid residues are indicated as (+).

### Example 20

### Isolation and Characterization of Murine Proliferin Analog I cDNAs

In this example, the isolation and characterization of the gene encoding murine Proliferin analog I is described.

The murine gene was discovered by analysis of an EST database (a GenBank™ search of the dbEST database) using human growth hormone as a probe. dbEST clone aa014234 was identified and subsequently obtained from Research Genetics (Huntsville, Ala.). This EST represents a mouse placenta derived clone which contains an ATG translation initiation codon and is annotated as mouse proliferin-related protein. BlastP searching (BLAST™ searching utilizing an amino acid sequence against a protein database), using the translation product (frame 1) of this sequence, revealed homology to proteins belonging to the prolactin-growth hormone superfamily. The mouse clone was fully sequenced (SEQ ID NO:53).

### Example 21

### Distribution of Proliferin Analog I mRNA in Mouse and Human Tissues

Clone aa014234 was digested with a NotI and an EcoRI enzyme to excise the fragment from the vector and this fragment was used as a probe for Northern blots. The fragment was labeled using the Prime It kit from Stratagene (La Jolla, Calif.) and then hybridized to multi-tissue northern blots from Clontech (Palo Alto, Calif.) as recommended by the manufacturer. Three blots were used: a human (7760-1), a human immune system II (7768-1) and a mouse embryo (7763-1). A band of about 1 Kb was detected in tissue from a mouse day 7 embryo (see FIG. 16B) under low stringency hybridization conditions (hybridization at 68° C., wash in 2×SSC, 0.05% SDS at 68° C. for 20 minutes). A band of the same size (1 Kb) was detected in human placenta tissue (see FIG. 16A) and in human fetal liver (see FIG. 16C) under similar low stringency hybridization conditions (hybridization at 50° C., wash in 2×SSC, 0.05% SDS at room temperature for 20 minutes and at 50° C. for 20 more minutes).

### Example 22

### Screening for the Human cDNA and Genomic DNA

A human placenta library (Clontech), as well as a human fetal liver library (Clontech) and a human genomic library (Stratagene) are screened using the same probe that was used in the northern blot experiment described above. Hybridization is performed under low stringency conditions. Briefly, the hybridization is performed overnight at 45° C., in Church buffer (7% SDS, 250 mM NaHP04, 2 μM EDTA), and is followed by washing of the filters in 2×SSC, 1% SDS. The blots are exposed to film at −80° C. for 5 hours. Positive clones are isolated and sequenced using art known techniques.

### Example 23

### Expression of EDIRF, MTR-1, LSP-1, TAP-1, and PA-I in Bacterial Cells

In this example, EDIRF, MTR-1, LSP-1, TAP-1, or PA-I is expressed as a recombinant glutathione-S-transferase (GST) fusion polypeptide in E. coli and the fusion polypeptide is isolated and characterized. Specifically, DIRF, MTR-1, LSP-1, TAP-1, or PA-I is fused to GST and this fusion polypeptide is expressed in E. coli, e.g., strain PEB199. Expression of the DIRF, MTR-1, LSP-1, TAP-1, or PA-I fusion protein in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 24

### Expression of Recombinant EDIRF, MTR-1, LSP-1, TAP-1, and PA-I Protein in COS Cells

To express the EDIRF, MTR-1, LSP-1, TAP-1, or PA-I gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire EDIRF, MTR-1, LSP-1, TAP-1, or PA-I protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the EDIRF, MTR-1, LSP-1, TAP-1, or PA-I DNA sequence is amplified by PCR using two primers. The 5′ primer contains the restriction site of interest followed by approximately twenty nucleotides of the EDIRF, MTR-1, LSP-1, TAP-1, or PA-I coding sequence starting from the initiation codon; the 3′ end sequence contains complementary sequences to the other restriction site of interest, a translation stop codon, the HA tag or FLAG tag and the last 20 nucleotides of the EDIRF, MTR-1, LSP-1, TAP-1, or PA-I coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the EDIRF, MTR-1, LSP-1, TAP-1, or PA-I gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the EDIRF, MTR-1, LSP-1, TAP-1, or PA-I -pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the EDIRF, MTR-1, LSP-1, TAP-1, or PA-I polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the EDIRF, MTR-1, LSP-1, TAP-1, or PA-I coding sequence is cloned directly into the polylinker of the pCDNA/Amp vector using the appropriate restriction sites. The resulting plasmid is transfected into COS cells in the manner described above, and the expression of the EDIRF, MTR-1, LSP-1, TAP-1, or PA-I polypeptide is detected by radiolabelling and immunoprecipitation using a EDIRF, MTR-1, LSP-1, TAP-1, or PA-I specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 90% identical to the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof;
b) a nucleic acid molecule comprising at least 15 nucleotide residues and having a nucleotide sequence identical to at least 15 consecutive nucleotide residues of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof;
c) a nucleic acid molecule which encodes a polypeptide comprising the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554;
d) a nucleic acid molecule which encodes a fragment of a polypeptide comprising the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554, wherein the fragment comprises at least 10 consecutive amino acid residues of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554; and
d) a nucleic acid molecule which encodes a fragment of a polypeptide comprising the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554, wherein the fragment comprises consecutive amino acid residues corresponding to at least half of the full length of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554; and
f) a nucleic acid molecule which encodes a naturally occurring allelic variant of a polypeptide comprising the amino acid sequence of any of SEQ ID NO:2, 4, 6, 35, 44, 50, or 54, wherein the nucleic acid molecule hybridizes with a nucleic acid molecule consisting of the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof under stringent conditions.

2. The isolated nucleic acid molecule of claim 1, which is selected from the group consisting of:
a) a nucleic acid having the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof; and
b) a nucleic acid molecule which encodes a polypeptide having the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554;
b) a naturally occurring allelic variant of a polypeptide comprising the amino acid sequence of any of SEQ ID NO:2, 4, 6, 35, 44, 50, or 54, wherein the polypeptide is encoded by a nucleic acid molecule which hybridizes with a nucleic acid molecule consisting of the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof.

9. The isolated polypeptide of claim 8 having the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554;
b) a polypeptide comprising a fragment of the amino acid sequence of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554, wherein the fragment comprises at least 10 contiguous amino acids of any of SEQ ID NO:2, 4, 6, 35, 44, 50, or 54, or the amino acid sequence encoded by the nucleotide sequence of any of the clones deposited as ATCC Accession numbers 98613, 98614, 98550, or 98554; and
c) a naturally occurring allelic variant of a polypeptide comprising the amino acid sequence of any of SEQ ID NO:2, 4, 6, 35, 44, 50, or 54, or a complement thereof, wherein the polypeptide is encoded by a nucleic acid molecule which hybridizes with a nucleic acid molecule consisting of the nucleotide sequence of any of SEQ ID NO:1, 3, 5, 7, 8, 9, 34, 36, 43, 45, 49, 51, 53 or 55, or the nucleotide sequence of any of the clones deposited as ATCC Accession numbers 98613, 98614, 98550, or 98554, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for an activity characteristic of the polypeptide.

21. A method for modulating the activity of a polypeptide of claim 8 comprising contacting a polypeptide or a cell expressing a polypeptide of claim 8 with a compound which binds with the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.

23. An antibody substance which selectively binds with the polypeptide of claim 8.

24. A method of making an antibody substance which selectively binds with the polypeptide of claim 8, the method comprising providing the polypeptide to an immunocompetent vertebrate and thereafter harvesting from the vertebrate blood or serum comprising the antibody substance.

25. A method of making an antibody substance which selectively binds with the polypeptide of claim 8, the method comprising contacting the polypeptide with a plurality of particles which individually comprise an antibody substance and a a nucleic acid encoding the antibody substance, segregating a particle which selectively binds with the polypeptide, and expressing the antibody substance from the nucleic acid of the segregated particle.

26. The isolated nucleic acid of claim 1, wherein the isolated nucleic acid comprises a portion having the nucleotide sequence SEQ ID NO:1.

27. The isolated nucleic acid of claim 1, wherein the isolated nucleic acid comprises a portion having the nucleotide sequence SEQ ID NO:3.

28. The isolated nucleic acid of claim 1, wherein the isolated nucleic acid comprises a portion having the nucleotide sequence SEQ ID NO:5.

29. The isolated nucleic acid of claim 1, wherein the isolated nucleic acid comprises a portion having the nucleotide sequence SEQ ID NO:7.

30. The isolated nucleic acid of claim 1, wherein the isolated nucleic acid comprises a portion having the nucleotide sequence SEQ ID NO:8.

31. The isolated nucleic acid of claim 1, wherein the isolated nucleic acid comprises a portion having the nucleotide sequence SEQ ID NO:9.

32. The isolated nucleic acid of claim 1, wherein the isolated nucleic acid comprises a portion having the nucleotide sequence SEQ ID NO:34.

33. The isolated nucleic acid of claim 1, wherein the isolated nucleic acid comprises a portion having the nucleotide sequence SEQ ID NO:36.

34. The isolated nucleic acid of claim 1, wherein the isolated nucleic acid comprises a portion having the nucleotide sequence SEQ ID NO:43.

35. The isolated nucleic acid of claim 1, wherein the isolated nucleic acid comprises a portion having the nucleotide sequence SEQ ID NO:45.

36. The isolated nucleic acid of claim 1, wherein the isolated nucleic acid comprises a portion having the nucleotide sequence SEQ ID NO:49.

37. The isolated nucleic acid of claim 1, wherein the isolated nucleic acid comprises a portion having the nucleotide sequence SEQ ID NO:51.

38. The isolated nucleic acid of claim 1, wherein the isolated nucleic acid comprises a portion having the nucleotide sequence SEQ ID NO:53.

39. The isolated nucleic acid of claim 1, wherein the isolated nucleic acid comprises a portion having the nucleotide sequence SEQ ID NO:55.

40. The isolated polypeptide of claim 8, wherein the amino acid sequence of the isolated polypeptide is SEQ ID NO:2.

41. The isolated polypeptide of claim 8, wherein the amino acid sequence of the isolated polypeptide is SEQ ID NO:4.

42. The isolated polypeptide of claim 8, wherein the amino acid sequence of the isolated polypeptide is SEQ ID NO:6.

43. The isolated polypeptide of claim 8, wherein the amino acid sequence of the isolated polypeptide is SEQ ID NO:35.

44. The isolated polypeptide of claim 8, wherein the amino acid sequence of the isolated polypeptide is SEQ ID NO:44.

45. The isolated polypeptide of claim 8, wherein the amino acid sequence of the isolated polypeptide is SEQ ID NO:50.

46. The isolated polypeptide of claim 8, wherein the amino acid sequence of the isolated polypeptide is SEQ ID NO:54.
